SEQUENCE LISTING (106 sequences)

SEQ ID NO: 1 - protein, 686 aa, Artificial Sequence
  codon optimized HCV NS3-NS4A (active)
MAPITAYAQQTRGLLGCIITSLTGRDKNQVEGEVQIVSTAAQTFLATCINGVCWTVYHGAGTRTIASPKGPVIQMYTNVDQDLVGWPAPQGARSLTPCTCGSSDLYLVTRHADVIPVRRRGDGRGSLLSPRPISYLKGSSGGPLLCPAGHAVGIFRAAVCTRGVAKAVDFIPVESLETTMRSPVFSDNSSPPAVPQSYQVAHLHAPTGSGKSTKVPAAYAAQGYKVLVLNPSVAATMGFGAYMSKAHGIDPNIRTGVRTITTGSPITYSTYGKFLADGGCSGGAYDIIICDECHSTDATSILGIGTVLDQAETAGARLTVLATATPPGSVTVPHPNIEEVALSTTGEIPFYGKAIPLEAIKGGRHLIFCHSKKKCDELAAKLVALGVNAVAYYRGLDVSVIPTSGDVVVVATDALMTGFTGDFDSVIDCNTCVTQTVDFSLDPTFTIETITLPQDAVSRTQRRGRTGRGKPGIYRFVAPGERPSGMFDSSVLCECYDAGCAWYELTPAETTVRLRAYMNTPGLPVCQDHLEFWEGVFTGLTHIDAHFLSQTKQSGENLPYLVAYQATVCARAQAPPPSWDQMWKCLIRLKPTLHGPTPLLYRLGAVQNEVTLTHPVTKYIMTCMSADLEVVTSTWVLVGGVLAALAAYCLSTGCVVIVGRIVLSGKPAIIPDREVLYREFDEMEEC

SEQ ID NO: 2 - DNA, 2061 bp, Artificial Sequence
  codon optimized HCV NS3/4a (active)
atggcccccatcaccgcctacgcccagcagacccgcggcctgctgggctgcatcatcaccagcctgaccggccgcgacaagaaccaggtggagggcgaggtgcagatcgtgagcaccgccgcccagaccttcctggccacctgcatcaacggcgtgtgctggaccgtgtaccacggcgccggcacccgcaccatcgccagccccaagggccccgtgatccagatgtacaccaacgtggaccaggacctggtgggctggcccgccccccagggcgcccgcagcctgaccccctgcacctgcggcagcagcgacctgtacctggtgacccgccacgccgacgtgatccccgtgcgccgccgcggcgacggccgcggcagcctgctgagcccccgccccatcagctacctgaagggcagcagcggcggccccctgctgtgccccgccggccacgccgtgggcatcttccgcgccgccgtgtgcacccgcggcgtggccaaggccgtggacttcatccccgtggagagcctggagaccaccatgcgcagccccgtgttcagcgacaacagcagcccccccgccgtgccccagagctaccaggtggcccacctgcacgcccccaccggcagcggcaagagcaccaaggtgcccgccgcctacgccgcccagggctacaaggtgctggtgctgaaccccagcgtggccgccaccatgggcttcggcgcctacatgagcaaggcccacggcatcgaccccaacatccgcaccggcgtgcgcaccatcaccaccggcagccccatcacctacagcacctacggcaagttcctggccgacggcggctgcagcggcggcgcctacgacatcatcatctgcgacgagtgccacagcaccgacgccaccagcatcctgggcatcggcaccgtgctggaccaggccgagaccgccggcgcccgcctgaccgtgctggccaccgccaccccccccggcagcgtgaccgtgccccaccccaacatcgaggaggtggccctgagcaccaccggcgagatccccttctacggcaaggccatccccctggaggccatcaagggcggccgccacctgatcttctgccacagcaagaagaagtgcgacgagctggccgccaagctggtggccctgggcgtgaacgccgtggcctactaccgcggcctggacgtgagcgtgatccccaccagcggcgacgtggtggtggtggccaccgacgccctgatgaccggcttcaccggcgacttcgacagcgtgatcgactgcaacacctgcgtgacccagaccgtggacttcagcctggaccccaccttcaccatcgagaccatcaccctgccccaggacgccgtgagccgcacccagcgccgcggccgcaccggccgcggcaagcccggcatctaccgcttcgtggcccccggcgagcgccccagcggcatgttcgacagcagcgtgctgtgcgagtgctacgacgccggctgcgcctggtacgagctgacccccgccgagaccaccgtgcgcctgcgcgcctacatgaacacccccggcctgcccgtgtgccaggaccacctggagttctgggagggcgtgttcaccggcctgacccacatcgacgcccacttcctgagccagaccaagcagagcggcgagaacctgccctacctggtggcctaccaggccaccgtgtgcgcccgcgcccaggcccccccccccagctgggaccagatgtggaagtgcctgatccgcctgaagcccaccctgcacggccccacccccctgctgtaccgcctgggcgccgtgcagaacgaggtgaccctgacccaccccgtgaccaagtacatcatgacctgcatgagcgccgacctggaggtggtgaccagcacctgggtgctggtgggcggcgtgctggccgccctggccgcctactgcctgagcaccggctgcgtggtgatcgtgggccgcatcgtgctgagcggcaagcccgccatcatccccgaccgcgaggtgctgtaccgcgagttcgacgagatggaggagtgctga

SEQ ID NO: 3 - protein, 18 aa, HCV
  HCV NS3/4A junction
SADLEVVTSTWVLVGGVL

SEQ ID NO: 4 - DNA, 54 bp, HCV
  misc_feature (0)...(0): HCV NS3/4A junction
agcgccgacctggaggtggtgaccagcacctgggtgctggtgggcggcgtgctg

SEQ ID NO: 5 - protein, 16 aa, HCV
  HCV NS4A/B junction
DEMEECSQHLPYIEQG

SEQ ID NO: 6 - DNA, 48 bp, HCV
  misc_feature (0)...(0): HCV NS4A/B junction
gacgagatggaggagtgcagccagcacctgccctacatcgagcagggc

SEQ ID NO: 7 - protein, 449 aa, Artificial Sequence
  Codon optimized HCV NS5A genotype 1b
MGSGSWLRDVWDWICTVLTDFKTWLQSKLLPKLPGVPFFSCQRGYKGVWRGDGIMQTTCPCGAQITGHVKNGSMRIVGPKTCSNTWHGTFPINAYTTGPCTPSPAPNYSRALWRVAAEEYVEITRVGDFHYVTGMTTDNVKCPCQVPAPEFFTELDGVRLHRYAPACRPLLREDVTFQVGLNQYLVGSQLPCEPEPDVAVLTSMLTDPSHITAETAKRRLARGSPPSLASSSASQLSAPSLKATCTTHHDSPDADLIEANLLWRQEMGGNITRVESENKVVILDSFDPLRAEEDEREVSVAAEILRKSKKFPPALPIWARPDYNPPLLESWKSPDYVPPAVHGCPLPPTTGPPIPPPRKKRTVVLTESTVSSALAELATKTFGSSGSSAVDSGTATAPPDQTSDDGDKESDIESYSSMPPLEGEPGDPDLSDGSWSTVSGEAGDDIVCC

SEQ ID NO: 8 - DNA, 1350 bp, Artificial Sequence
  Codon optimized HCV NS5A genotype 1b
atgggatctggaagctggctgagggatgtttgggattggatttgtaccgtcctcaccgacttcaaaacctggctccagtccaagctgctgccaaagctgcccggagtgccattcttctcctgtcagaggggctataaaggcgtgtggaggggcgatggaattatgcagactacttgcccctgtggagctcaaattactgggcacgttaagaatggctccatgcggattgttggcccaaaaacctgttccaacacctggcacggaaccttccctattaacgcttacaccaccggaccttgcactccttcccccgcacctaattattcccgggctctctggcgggtggcagcagaggaatatgtcgaaattaccagagtcggcgacttccactacgtcacaggaatgactacagacaacgttaaatgtccctgccaagtgcccgctccagagttctttaccgaactcgacggggttaggctccacagatacgcacccgcctgccggccactgctgcgggaagacgtcacattccaggtcgggctgaaccagtacctggtgggctctcagctgccttgtgagcctgagcccgacgtggcagttctcaccagcatgctcaccgatcctagccacatcaccgctgagacagccaaacgccgcctggctagagggtcccctccctctctggccagctccagcgctagccagctctccgcaccaagcctgaaagccacatgcactacacaccacgatagccccgacgcagacctgattgaagccaacctcctctggagacaggaaatgggcggaaacatcactagggtcgaatccgagaataaagtggttattctggatagcttcgacccactcagggcagaggaagatgagagagaggttagcgtggccgctgagattctccgcaagtccaaaaagttccctcccgcactgcccatttgggcaaggcccgattacaatcctccactgctcgagagctggaagtcccctgactacgtgccaccagccgtccacggatgccctctgccccctaccacaggaccaccaattccaccccctagaaagaaacggaccgtggttctgactgagtccaccgtgtcctctgcactcgctgagctggcaaccaagacctttggatccagcggatcctccgcagtcgactccggcaccgctaccgccccacccgatcaaacctctgacgatggagacaaggagagcgatattgagtcctattccagcatgcccccactcgagggagaacccggcgaccccgacctgagcgatgggtcctggagcactgtgagcggggaagcaggggacgacattgtctgttgctga

SEQ ID NO: 9 - protein, 183 aa, Artificial Sequence
  Codon optimized HBV HBcAg subtype ayw
MDIDPYKEFGATVELLSFLPSDFFPSVRDLLDTASALYREALESPEHCSPHHTALRQAILCWGELMTLATWVGVNLEDPASRDLVVSYVNTNMGLKFRQLLWFHISCLTFGRETVIEYLVSFGVWIRTPPAYRPPNAPILSTLPETTVVRRRGRSPRRRTPSPRRRRSQSPRRRRSQSRESQC

SEQ ID NO: 10 - DNA, 552 bp, Artificial Sequence
  Codon optimized HBV HBcAg subtype ayw
atggacatcgacccctacaaggagttcggcgccaccgtggagctgctgagcttcctgcccagcgacttcttccccagcgtgagagacctgctggacaccgccagcgccctgtacagagaggccctggagagccccgagcactgcagcccccaccacaccgccctgagacaggccatcctgtgctggggcgagctgatgaccctggccacctgggtgggcgtgaacctggaggaccccgccagcagagacctggtggtgagctacgtgaacaccaacatgggcctgaagttcagacagctgctgtggttccacatcagctgcctgaccttcggcagagagaccgtgatcgagtacctggtgagcttcggcgtgtggatcagaaccccccccgcctacagaccccccaacgcccccatcctgagcaccctgcccgagaccaccgtggtgagaagaagaggcagaagccccagaagaagaacccccagccccagaagaagaagaagccagagccccagaagaagaagaagccagagcagagagagccagtgctag

SEQ ID NO: 11 - protein, 212 aa, Artificial Sequence
  Codon optimized HBV HBeAg subtype ayw
MQLFHLCLIISCSCPTVQASKLCLGWLWGMDIDPYKEFGATAELLSFLPSDFFPSVRDLLDTASALYREALESPEHCSPHHTALRQAILCWGELMTLATWVGVNLEDPASRDLVVSYVNTNMGLKFRQLLWFHISCLTFGRETVIEYLVSFGVWIRTPPAYRPPNAPILSTLPETTVVRRRGRSPRRRTPSPRRRRSQSPRRRRSQSRESQC

SEQ ID NO: 12 - DNA, 639 bp, Artificial Sequence
  Codon optimized HBV HBeAg subtype ayw
atgcagctgttccacctgtgcctgatcatcagctgcagctgccccaccgtgcaggccagcaagctgtgcctgggctggctgtggggcatggacatcgacccctacaaggagttcggcgccaccgccgagctgctgagcttcctgcccagcgacttcttccccagcgtgagagacctgctggacaccgccagcgccctgtacagagaggccctggagagccccgagcactgcagcccccaccacaccgccctgagacaggccatcctgtgctggggcgagctgatgaccctggccacctgggtgggcgtgaacctggaggaccccgccagcagagacctggtggtgagctacgtgaacaccaacatgggcctgaagttcagacagctgctgtggttccacatcagctgcctgaccttcggcagagagaccgtgatcgagtacctggtgagcttcggcgtgtggatcagaaccccccccgcctacagaccccccaacgcccccatcctgagcaccctgcccgagaccaccgtggtgagaagaagaggcagaagccccagaagaagaacccccagccccagaagaagaagaagccagagccccagaagaagaagaagccagagcagagagagccagtgctag

SEQ ID NO: 13 - protein, 212 aa, Artificial Sequence
  Codon optimized HBV HBeAg subtype ayw
MQLFHLCLIISCSCPTVQASKLCLGWLWGLDIDPYKEFGATAELLSFLPSDFFPSVRDLLDTASALYREALESPEHCSPHHTALRQAILCWGELMTLATWVGVNLEDPASRDLVVSYVNTNMGLKFRQLLWFHISCLTFGRETVIEYLVSFGVWIRTPPAYRPPNAPILSTLPETTVVRRRGRSPRRRTPSPRRRRSQSPRRRRSQSRESQC

SEQ ID NO: 14 - DNA, 639 bp, Artificial Sequence
  Codon optimized HBV HBeAg subtype ayw
atgcagctgttccacctgtgcctgatcatcagctgcagctgccccaccgtgcaggccagcaagctgtgcctgggctggctgtggggcctggacatcgacccctacaaggagttcggcgccaccgccgagctgctgagcttcctgcccagcgacttcttccccagcgtgagagacctgctggacaccgccagcgccctgtacagagaggccctggagagccccgagcactgcagcccccaccacaccgccctgagacaggccatcctgtgctggggcgagctgatgaccctggccacctgggtgggcgtgaacctggaggaccccgccagcagagacctggtggtgagctacgtgaacaccaacatgggcctgaagttcagacagctgctgtggttccacatcagctgcctgaccttcggcagagagaccgtgatcgagtacctggtgagcttcggcgtgtggatcagaaccccccccgcctacagaccccccaacgcccccatcctgagcaccctgcccgagaccaccgtggtgagaagaagaggcagaagccccagaagaagaacccccagccccagaagaagaagaagccagagccccagaagaagaagaagccagagcagagagagccagtgctag

SEQ ID NO: 15 - protein, 386 aa, Artificial Sequence
  Codon optimized Ovalbumin
MGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSP

SEQ ID NO: 16 - DNA, 1161 bp, Artificial Sequence
  Codon optimized Ovalbumin
atgggcagcatcggcgccgccagcatggagttctgcttcgacgtgttcaaggagctgaaggtgcaccacgccaacgagaacatcttctactgccccatcgccatcatgagcgccctggccatggtgtacctgggcgccaaggacagcacccgcacccagatcaacaaggtggtgcgcttcgacaagctgcccggcttcggcgacagcatcgaggcccagtgcggcaccagcgtgaacgtgcacagcagcctgcgcgacatcctgaaccagatcaccaagcccaacgacgtgtacagcttcagcctggccagccgcctgtacgccgaggagcgctaccccatcctgcccgagtacctgcagtgcgtgaaggagctgtaccgcggcggcctggagcccatcaacttccagaccgccgccgaccaggcccgcgagctgatcaacagctgggtggagagccagaccaacggcatcatccgcaacgtgctgcagcccagcagcgtggacagccagaccgccatggtgctggtgaacgccatcgtgttcaagggcctgtgggagaaggccttcaaggacgaggacacccaggccatgcccttccgcgtgaccgagcaggagagcaagcccgtgcagatgatgtaccagatcggcctgttccgcgtggccagcatggccagcgagaagatgaagatcctggagctgcccttcgccagcggcaccatgagcatgctggtgctgctgcccgacgaggtgagcggcctggagcagctggagagcatcatcaacttcgagaagctgaccgagtggaccagcagcaacgtgatggaggagcgcaagatcaaggtgtacctgccccgcatgaagatggaggagaagtacaacctgaccagcgtgctgatggccatgggcatcaccgacgtgttcagcagcagcgccaacctgagcggcatcagcagcgccgagagcctgaagatcagccaggccgtgcacgccgcccacgccgagatcaacgaggccggccgcgaggtggtgggcagcgccgaggccggcgtggacgccgccagcgtgagcgaggagttccgcgccgaccaccccttcctgttctgcatcaagcacatcgccaccaacgccgtgctgttcttcggccgctgcgtgagcccctaa

SEQ ID NO: 17 - protein, 160 aa, Artificial Sequence
  Codon optimized BetvI (Birch)
MGVFNYETETTSVIPAARLFKAFILDGDNLVPKVAPQAISSVENIEGNGGPGTIKKINFPEGFPFKYVKDRVDEVDHTNFKYNYSVIEGGPVGDTLEKISNEIKIVATPDGGCVLKISNKYHTKGNHEVKAEQVKASKEMGETLLRAVESYLLAHSDAYN

SEQ ID NO: 18 - DNA, 483 bp, Artificial Sequence
  Codon optimized BetvI (Birch)
atgggcgtcttcaattacgaaaccgagacaactagtgtgatcccagctgcacgcctttttaaggccttcattcttgatggagacaaccttgtgccaaaggtggctccccaagctattagtagcgtcgaaaacatcgagggaaatggcggaccgggaaccatcaaaaagatcaactttccggagggcttccctttcaagtacgttaaggacagggttgatgaagttgatcatacaaattttaagtacaactactccgtgatcgagggcggccccgttggagatactctggaaaagatcagcaacgagattaagatcgtggctacacccgatggcgggtgtgtgcttaagatctccaacaaatatcataccaaagggaaccacgaggttaaggctgaacaggtgaaggcatcaaaggagatgggagagaccctcctccgagcggtggagtcttacttgctcgcacacagcgatgcttacaattag

SEQ ID NO: 19 - protein, 262 aa, Artificial Sequence
  Codon optimized Stork HBcAg
MDVNASRALANVYDLPDDFFPQIDDLVRDAKDALEPYWKAETIKKHVLIATHFVDLIEDFWQTTQGMSQIADALRAVIPPTTTPVPDGYLISHNEAQELPLNDLFVLQEERIVNFQPDYPITARIHTHLRVYTKLNEQALDKARRLLWWHYNCLLWGESNVTNYISRLRTWLSTPEKYRGKDAPTIEAITRPIQVAQGSRNQTKGVRKPRGLEPRRRKVKTTVVYGRRRSKSRGRRSSPSQRAGSPIPRNRENQSRSSSPRE

SEQ ID NO: 20 - DNA, 789 bp, Artificial Sequence
  Codon optimized Stork HBcAg
atggacgtgaacgccagcagagccctggccaacgtgtacgacctgcccgacgacttcttcccccagatcgacgacctggtgagagacgccaaggacgccctggagccctactggaaggccgagaccatcaagaagcacgtgctgatcgccacccacttcgtggacttgatcgaggacttctggcagaccacccagggcatgagccagatcgccgacgccctgagagccgtgatcccccccaccaccacccccgtgcccgacggctacctgatcagccacaacgaggcccaggagctccccctgaacgacctgttcgtgctgcaggaggagagaatcgtgaacttccagcccgactaccccatcaccgccagaattcacacccacctgagagtgtacaccaagctgaacgagcaggccctggacaaggccagaagactgctgtggtggcactacaactgcctgctgtggggcgagagcaacgtgaccaactacatcagcagactgagaacctggctgagcacccccgagaagtacagaggcaaggacgcccccaccatcgaggccatcaccagacccatccaggtggcccagggcagcagaaaccagaccaagggcgtgagaaagcccagaggcctggagcccagaagaagaaaggtgaagaccaccgtggtgtacggcagaagaagaagcaagagcagaggcagaagaagcagccccagccagagagccggcagccccatccccagaaacagagagaaccagagcagaagcagcagccccagagagtga

SEQ ID NO: 21 - protein, 262 aa, Artificial Sequence
  Codon optimized Heron HBcAg
MDVNASRALANVYDLPDDFFPQIDDLVRDAKDALEPYWKAETIKKHVLIATHFVDLIEDFWQTTQGMSQIADALRAVIPPTTVPVPEGFLITHSEAEELPLNDLFSLQEERIVNFQPDYPITARIHTHLRVYTKLNEQALDKARRLLWWHYNCLLWGEATVTNYISRLRTWLSTPEKYRGKDAPTIEAITRPIQVAQGGRNQTKGTRKPRGLEPRRRKVKTTVVYGRRRSKSRGRRSSPSQRAGSPLPRNRGNQTRSPSPRE

SEQ ID NO: 22 - DNA, 789 bp, Artificial Sequence
  Codon optimized Heron HBcAg
atggacgtgaacgccagcagagccctggccaacgtgtacgacctgcccgacgacttcttcccccagatcgacgacctggtgagagacgccaaggacgccctggagccctactggaaggccgagaccatcaagaagcacgtgctgatcgccacccacttcgtggacttgatcgaggacttctggcagaccacccagggcatgagccagatcgccgacgccctgagagccgtgatcccccccaccaccgtgcccgtgcccgagggcttcctgatcacccacagcgaggccgaggagctccccctgaacgacctgttcagtctgcaggaggagagaatcgtgaacttccagcccgactaccccatcaccgccagaattcacacccacctgagagtgtacaccaagctgaacgagcaggccctggacaaggccagaagactgctgtggtggcactacaactgcctgctgtggggcgaggccaccgtgaccaactacatcagcagactgagaacctggctgagcacccccgagaagtacagaggcaaggacgcccccaccatcgaggccatcaccagacccatccaggtggcccagggcggcagaaaccagaccaagggcaccagaaagcccagaggcctggagcccagaagaagaaaggtgaagaccaccgtggtgtacggcagaagaagaagcaagagcagaggcagaagaagcagccccagccagagagccggcagccccctgcccagaaacagaggcaaccagaccagaagccccagccccagagagtga

SEQ ID NO: 23 - protein, 948 aa, Artificial Sequence
  Codon optimized HCV NS3/4A-Stork core
MAPITAYAQQTRGLLGCIITSLTGRDKNQVEGEVQIVSTAAQTFLATCINGVCWTVYHGAGTRTIASPKGPVIQMYTNVDQDLVGWPAPQGARSLTPCTCGSSDLYLVTRHADVIPVRRRGDGRGSLLSPRPISYLKGSSGGPLLCPAGHAVGIFRAAVCTRGVAKAVDFIPVESLETTMRSPVFSDNSSPPAVPQSYQVAHLHAPTGSGKSTKVPAAYAAQGYKVLVLNPSVAATMGFGAYMSKAHGIDPNIRTGVRTITTGSPITYSTYGKFLADGGCSGGAYDIIICDECHSTDATSILGIGTVLDQAETAGARLTVLATATPPGSVTVPHPNIEEVALSTTGEIPFYGKAIPLEAIKGGRHLIFCHSKKKCDELAAKLVALGVNAVAYYRGLDVSVIPTSGDVVVVATDALMTGFTGDFDSVIDCNTCVTQTVDFSLDPTFTIETITLPQDAVSRTQRRGRTGRGKPGIYRFVAPGERPSGMFDSSVLCECYDAGCAWYELTPAETTVRLRAYMNTPGLPVCQDHLEFWEGVFTGLTHIDAHFLSQTKQSGENLPYLVAYQATVCARAQAPPPSWDQMWKCLIRLKPTLHGPTPLLYRLGAVQNEVTLTHPVTKYIMTCMSADLEVVTPTWVLVGGVLAALAAYCLSTGCVVIVGRIVLSGKPAIIPDREVLYREFDEMEECMDVNASRALANVYDLPDDFFPQIDDLVRDAKDALEPYWKAETIKKHVLIATHFVDLIEDFWQTTQGMSQIADALRAVIPPTTTPVPDGYLISHNEAQELPLNDLFVLQEERIVNFQPDYPITARIHTHLRVYTKLNEQALDKARRLLWWHYNCLLWGESNVTNYISRLRTWLSTPEKYRGKDAPTIEAITRPIQVAQGSRNQTKGVRKPRGLEPRRRKVKTTVVYGRRRSKSRGRRSSPSQRAGSPIPRNRENQSRSSSPRE

SEQ ID NO: 24 - DNA, 2847 bp, Artificial Sequence
  Codon optimized HCV NS3/4A-Stork HBcAg
atggcccccatcaccgcctacgcccagcagacccgcggcctgctgggctgcatcatcaccagcctgaccggccgcgacaagaaccaggtggagggcgaggtgcagatcgtgagcaccgccgcccagaccttcctggccacctgcatcaacggcgtgtgctggaccgtgtaccacggcgccggcacccgcaccatcgccagccccaagggccccgtgatccagatgtacaccaacgtggaccaggacctggtgggctggcccgccccccagggcgcccgcagcctgaccccctgcacctgcggcagcagcgacctgtacctggtgacccgccacgccgacgtgatccccgtgcgccgccgcggcgacggccgcggcagcctgctgagcccccgccccatcagctacctgaagggcagcagcggcggccccctgctgtgccccgccggccacgccgtgggcatcttccgcgccgccgtgtgcacccgcggcgtggccaaggccgtggacttcatccccgtggagagcctggagaccaccatgcgcagccccgtgttcagcgacaacagcagcccccccgccgtgccccagagctaccaggtggcccacctgcacgcccccaccggcagcggcaagagcaccaaggtgcccgccgcctacgccgcccagggctacaaggtgctggtgctgaaccccagcgtggccgccaccatgggcttcggcgcctacatgagcaaggcccacggcatcgaccccaacatccgcaccggcgtgcgcaccatcaccaccggcagccccatcacctacagcacctacggcaagttcctggccgacggcggctgcagcggcggcgcctacgacatcatcatctgcgacgagtgccacagcaccgacgccaccagcatcctgggcatcggcaccgtgctggaccaggccgagaccgccggcgcccgcctgaccgtgctggccaccgccaccccccccggcagcgtgaccgtgccccaccccaacatcgaggaggtggccctgagcaccaccggcgagatccccttctacggcaaggccatccccctggaggccatcaagggcggccgccacctgatcttctgccacagcaagaagaagtgcgacgagctggccgccaagctggtggccctgggcgtgaacgccgtggcctactaccgcggcctggacgtgagcgtgatccccaccagcggcgacgtggtggtggtggccaccgacgccctgatgaccggcttcaccggcgacttcgacagcgtgatcgactgcaacacctgcgtgacccagaccgtggacttcagcctggaccccaccttcaccatcgagaccatcaccctgccccaggacgccgtgagccgcacccagcgccgcggccgcaccggccgcggcaagcccggcatctaccgcttcgtggcccccggcgagcgccccagcggcatgttcgacagcagcgtgctgtgcgagtgctacgacgccggctgcgcctggtacgagctgacccccgccgagaccaccgtgcgcctgcgcgcctacatgaacacccccggcctgcccgtgtgccaggaccacctggagttctgggagggcgtgttcaccggcctgacccacatcgacgcccacttcctgagccagaccaagcagagcggcgagaacctgccctacctggtggcctaccaggccaccgtgtgcgcccgcgcccaggcccccccccccagctgggaccagatgtggaagtgcctgatccgcctgaagcccaccctgcacggccccacccccctgctgtaccgcctgggcgccgtgcagaacgaggtgaccctgacccaccccgtgaccaagtacatcatgacctgcatgagcgccgacctggaggtggtgacccccacctgggtgctggtgggcggcgtgctggccgccctggccgcctactgcctgagcaccggctgcgtggtgatcgtgggccgcatcgtgctgagcggcaagcccgccatcatccccgaccgcgaggtgctgtaccgcgagttcgacgagatggaggagtgcatggacgtgaacgccagcagagccctggccaacgtgtacgacctgcccgacgacttcttcccccagatcgacgacctggtgagagacgccaaggacgccctggagccctactggaaggccgagaccatcaagaagcacgtgctgatcgccacccacttcgtggacttgatcgaggacttctggcagaccacccagggcatgagccagatcgccgacgccctgagagccgtgatcccccccaccaccacccccgtgcccgacggctacctgatcagccacaacgaggcccaggagctccccctgaacgacctgttcgtgctgcaggaggagagaatcgtgaacttccagcccgactaccccatcaccgccagaattcacacccacctgagagtgtacaccaagctgaacgagcaggccctggacaaggccagaagactgctgtggtggcactacaactgcctgctgtggggcgagagcaacgtgaccaactacatcagcagactgagaacctggctgagcacccccgagaagtacagaggcaaggacgcccccaccatcgaggccatcaccagacccatccaggtggcccagggcagcagaaaccagaccaagggcgtgagaaagcccagaggcctggagcccagaagaagaaaggtgaagaccaccgtggtgtacggcagaagaagaagcaagagcagaggcagaagaagcagccccagccagagagccggcagccccatccccagaaacagagagaaccagagcagaagcagcagccccagagagtga

SEQ ID NO: 25 - protein, 948 aa, Artificial Sequence
  Codon optimized mutant NS3/4A-Stork HBcAg
MAPITAYAQQTRGLLGCIITSLTGRDKNQVEGEVQIVSTAAQTFLATCINGVCWTVYAGAGTRTIASPKGPVIQMYTNVDQALVGWPAPQGARSLTPCTCGSSDLYLVTRHADVIPVRRRGDGRGSLLSPRPISYLKGSSGGPLLCPAGHAVGIFRAAVCTRGVAKAVDFIPVESLETTMRSPVFSDNSSPPAVPQSYQVAHLHAPTGSGKSTKVPAAYAAQGYKVLVLNPSVAATMGFGAYMSKAHGIDPNIRTGVRTITTGSPITYSTYGKFLADGGCSGGAYDIIICDECHSTDATSILGIGTVLDQAETAGARLTVLATATPPGSVTVPHPNIEEVALSTTGEIPFYGKAIPLEAIKGGRHLIFCHSKKKCDELAAKLVALGVNAVAYYRGLDVSVIPTSGDVVVVATDALMTGFTGDFDSVIDCNTCVTQTVDFSLDPTFTIETITLPQDAVSRTQRRGRTGRGKPGIYRFVAPGERPSGMFDSSVLCECYDAGCAWYELTPAETTVRLRAYMNTPGLPVCQDHLEFWEGVFTGLTHIDAHFLSQTKQSGENLPYLVAYQATVCARAQAPPPSWDQMWKCLIRLKPTLHGPTPLLYRLGAVQNEVTLTHPVTKYIMTCMSADLEVVTSTWVLVGGVLAALAAYCLSTGCVVIVGRIVLSGKPAIIPDREVLYREFDEMEECMDVNASRALANVYDLPDDFFPQIDDLVRDAKDALEPYWKAETIKKHVLIATHFVDLIEDFWQTTQGMSQIADALRAVIPPTTTPVPDGYLISHNEAQELPLNDLFVLQEERIVNFQPDYPITARIHTHLRVYTKLNEQALDKARRLLWWHYNCLLWGESNVTNYISRLRTWLSTPEKYRGKDAPTIEAITRPIQVAQGSRNQTKGVRKPRGLEPRRRKVKTTVVYGRRRSKSRGRRSSPSQRAGSPIPRNRENQSRSSSPRE

SEQ ID NO: 26 - DNA, 2847 bp, Artificial Sequence
  Codon optimized mutant NS3/4A-Stork HBcAg
atggcccccatcaccgcctacgcccagcagacccgcggcctgctgggctgcatcatcaccagcctgaccggccgcgacaagaaccaggtggagggcgaggtgcagatcgtgagcaccgccgcccagaccttcctggccacctgcatcaacggcgtgtgctggaccgtgtacgccggcgccggcacccgcaccatcgccagccccaagggccccgtgatccagatgtacaccaacgtggaccaggccctggtgggctggcccgccccccagggcgcccgcagcctgaccccctgcacctgcggcagcagcgacctgtacctggtgacccgccacgccgacgtgatccccgtgcgccgccgcggcgacggccgcggcagcctgctgagcccccgccccatcagctacctgaagggcagcagcggcggccccctgctgtgccccgccggccacgccgtgggcatcttccgcgccgccgtgtgcacccgcggcgtggccaaggccgtggacttcatccccgtggagagcctggagaccaccatgcgcagccccgtgttcagcgacaacagcagcccccccgccgtgccccagagctaccaggtggcccacctgcacgcccccaccggcagcggcaagagcaccaaggtgcccgccgcctacgccgcccagggctacaaggtgctggtgctgaaccccagcgtggccgccaccatgggcttcggcgcctacatgagcaaggcccacggcatcgaccccaacatccgcaccggcgtgcgcaccatcaccaccggcagccccatcacctacagcacctacggcaagttcctggccgacggcggctgcagcggcggcgcctacgacatcatcatctgcgacgagtgccacagcaccgacgccaccagcatcctgggcatcggcaccgtgctggaccaggccgagaccgccggcgcccgcctgaccgtgctggccaccgccaccccccccggcagcgtgaccgtgccccaccccaacatcgaggaggtggccctgagcaccaccggcgagatccccttctacggcaaggccatccccctggaggccatcaagggcggccgccacctgatcttctgccacagcaagaagaagtgcgacgagctggccgccaagctggtggccctgggcgtgaacgccgtggcctactaccgcggcctggacgtgagcgtgatccccaccagcggcgacgtggtggtggtggccaccgacgccctgatgaccggcttcaccggcgacttcgacagcgtgatcgactgcaacacctgcgtgacccagaccgtggacttcagcctggaccccaccttcaccatcgagaccatcaccctgccccaggacgccgtgagccgcacccagcgccgcggccgcaccggccgcggcaagcccggcatctaccgcttcgtggcccccggcgagcgccccagcggcatgttcgacagcagcgtgctgtgcgagtgctacgacgccggctgcgcctggtacgagctgacccccgccgagaccaccgtgcgcctgcgcgcctacatgaacacccccggcctgcccgtgtgccaggaccacctggagttctgggagggcgtgttcaccggcctgacccacatcgacgcccacttcctgagccagaccaagcagagcggcgagaacctgccctacctggtggcctaccaggccaccgtgtgcgcccgcgcccaggcccccccccccagctgggaccagatgtggaagtgcctgatccgcctgaagcccaccctgcacggccccacccccctgctgtaccgcctgggcgccgtgcagaacgaggtgaccctgacccaccccgtgaccaagtacatcatgacctgcatgagcgccgacctggaggtggtgaccagcacctgggtgctggtgggcggcgtgctggccgccctggccgcctactgcctgagcaccggctgcgtggtgatcgtgggccgcatcgtgctgagcggcaagcccgccatcatccccgaccgcgaggtgctgtaccgcgagttcgacgagatggaggagtgcatggacgtgaacgccagcagagccctggccaacgtgtacgacctgcccgacgacttcttcccccagatcgacgacctggtgagagacgccaaggacgccctggagccctactggaaggccgagaccatcaagaagcacgtgctgatcgccacccacttcgtggacttgatcgaggacttctggcagaccacccagggcatgagccagatcgccgacgccctgagagccgtgatcccccccaccaccacccccgtgcccgacggctacctgatcagccacaacgaggcccaggagctccccctgaacgacctgttcgtgctgcaggaggagagaatcgtgaacttccagcccgactaccccatcaccgccagaattcacacccacctgagagtgtacaccaagctgaacgagcaggccctggacaaggccagaagactgctgtggtggcactacaactgcctgctgtggggcgagagcaacgtgaccaactacatcagcagactgagaacctggctgagcacccccgagaagtacagaggcaaggacgcccccaccatcgaggccatcaccagacccatccaggtggcccagggcagcagaaaccagaccaagggcgtgagaaagcccagaggcctggagcccagaagaagaaaggtgaagaccaccgtggtgtacggcagaagaagaagcaagagcagaggcagaagaagcagccccagccagagagccggcagccccatccccagaaacagagagaaccagagcagaagcagcagccccagagagtga

SEQ ID NO: 27 - protein, 948 aa, Artificial Sequence
  Codon optimized NS3/4A-Stork HBcAg
MAPITAYAQQTRGLLGCIITSLTGRDKNQVEGEVQIVSTAAQTFLATCINGVCWTVYHGAGTRTIASPKGPVIQMYTNVDQDLVGWPAPQGARSLTPCTCGSSDLYLVTRHADVIPVRRRGDGRGSLLSPRPISYLKGSSGGPLLCPAGHAVGIFRAAVCTRGVAKAVDFIPVESLETTMRSPVFSDNSSPPAVPQSYQVAHLHAPTGSGKSTKVPAAYAAQGYKVLVLNPSVAATMGFGAYMSKAHGIDPNIRTGVRTITTGSPITYSTYGKFLADGGCSGGAYDIIICDECHSTDATSILGIGTVLDQAETAGARLTVLATATPPGSVTVPHPNIEEVALSTTGEIPFYGKAIPLEAIKGGRHLIFCHSKKKCDELAAKLVALGVNAVAYYRGLDVSVIPTSGDVVVVATDALMTGFTGDFDSVIDCNTCVTQTVDFSLDPTFTIETITLPQDAVSRTQRRGRTGRGKPGIYRFVAPGERPSGMFDSSVLCECYDAGCAWYELTPAETTVRLRAYMNTPGLPVCQDHLEFWEGVFTGLTHIDAHFLSQTKQSGENLPYLVAYQATVCARAQAPPPSWDQMWKCLIRLKPTLHGPTPLLYRLGAVQNEVTLTHPVTKYIMTCMSADLEVVTSTWVLVGGVLAALAAYCLSTGCVVIVGRIVLSGKPAIIPDREVLYREFDEMEECMDVNASRALANVYDLPDDFFPQIDDLVRDAKDALEPYWKAETIKKHVLIATHFVDLIEDFWQTTQGMSQIADALRAVIPPTTTPVPDGYLISHNEAQELPLNDLFVLQEERIVNFQPDYPITARIHTHLRVYTKLNEQALDKARRLLWWHYNCLLWGESNVTNYISRLRTWLSTPEKYRGKDAPTIEAITRPIQVAQGSRNQTKGVRKPRGLEPRRRKVKTTVVYGRRRSKSRGRRSSPSQRAGSPIPRNRENQSRSSSPRE

SEQ ID NO: 28 - DNA, 2847 bp, Artificial Sequence
  Codon optimized NS3/4A-Stork HBcAg
atggcccccatcaccgcctacgcccagcagacccgcggcctgctgggctgcatcatcaccagcctgaccggccgcgacaagaaccaggtggagggcgaggtgcagatcgtgagcaccgccgcccagaccttcctggccacctgcatcaacggcgtgtgctggaccgtgtaccacggcgccggcacccgcaccatcgccagccccaagggccccgtgatccagatgtacaccaacgtggaccaggacctggtgggctggcccgccccccagggcgcccgcagcctgaccccctgcacctgcggcagcagcgacctgtacctggtgacccgccacgccgacgtgatccccgtgcgccgccgcggcgacggccgcggcagcctgctgagcccccgccccatcagctacctgaagggcagcagcggcggccccctgctgtgccccgccggccacgccgtgggcatcttccgcgccgccgtgtgcacccgcggcgtggccaaggccgtggacttcatccccgtggagagcctggagaccaccatgcgcagccccgtgttcagcgacaacagcagcccccccgccgtgccccagagctaccaggtggcccacctgcacgcccccaccggcagcggcaagagcaccaaggtgcccgccgcctacgccgcccagggctacaaggtgctggtgctgaaccccagcgtggccgccaccatgggcttcggcgcctacatgagcaaggcccacggcatcgaccccaacatccgcaccggcgtgcgcaccatcaccaccggcagccccatcacctacagcacctacggcaagttcctggccgacggcggctgcagcggcggcgcctacgacatcatcatctgcgacgagtgccacagcaccgacgccaccagcatcctgggcatcggcaccgtgctggaccaggccgagaccgccggcgcccgcctgaccgtgctggccaccgccaccccccccggcagcgtgaccgtgccccaccccaacatcgaggaggtggccctgagcaccaccggcgagatccccttctacggcaaggccatccccctggaggccatcaagggcggccgccacctgatcttctgccacagcaagaagaagtgcgacgagctggccgccaagctggtggccctgggcgtgaacgccgtggcctactaccgcggcctggacgtgagcgtgatccccaccagcggcgacgtggtggtggtggccaccgacgccctgatgaccggcttcaccggcgacttcgacagcgtgatcgactgcaacacctgcgtgacccagaccgtggacttcagcctggaccccaccttcaccatcgagaccatcaccctgccccaggacgccgtgagccgcacccagcgccgcggccgcaccggccgcggcaagcccggcatctaccgcttcgtggcccccggcgagcgccccagcggcatgttcgacagcagcgtgctgtgcgagtgctacgacgccggctgcgcctggtacgagctgacccccgccgagaccaccgtgcgcctgcgcgcctacatgaacacccccggcctgcccgtgtgccaggaccacctggagttctgggagggcgtgttcaccggcctgacccacatcgacgcccacttcctgagccagaccaagcagagcggcgagaacctgccctacctggtggcctaccaggccaccgtgtgcgcccgcgcccaggcccccccccccagctgggaccagatgtggaagtgcctgatccgcctgaagcccaccctgcacggccccacccccctgctgtaccgcctgggcgccgtgcagaacgaggtgaccctgacccaccccgtgaccaagtacatcatgacctgcatgagcgccgacctggaggtggtgaccagcacctgggtgctggtgggcggcgtgctggccgccctggccgcctactgcctgagcaccggctgcgtggtgatcgtgggccgcatcgtgctgagcggcaagcccgccatcatccccgaccgcgaggtgctgtaccgcgagttcgacgagatggaggagtgcatggacgtgaacgccagcagagccctggccaacgtgtacgacctgcccgacgacttcttcccccagatcgacgacctggtgagagacgccaaggacgccctggagccctactggaaggccgagaccatcaagaagcacgtgctgatcgccacccacttcgtggacttgatcgaggacttctggcagaccacccagggcatgagccagatcgccgacgccctgagagccgtgatcccccccaccaccacccccgtgcccgacggctacctgatcagccacaacgaggcccaggagctccccctgaacgacctgttcgtgctgcaggaggagagaatcgtgaacttccagcccgactaccccatcaccgccagaattcacacccacctgagagtgtacaccaagctgaacgagcaggccctggacaaggccagaagactgctgtggtggcactacaactgcctgctgtggggcgagagcaacgtgaccaactacatcagcagactgagaacctggctgagcacccccgagaagtacagaggcaaggacgcccccaccatcgaggccatcaccagacccatccaggtggcccagggcagcagaaaccagaccaagggcgtgagaaagcccagaggcctggagcccagaagaagaaaggtgaagaccaccgtggtgtacggcagaagaagaagcaagagcagaggcagaagaagcagccccagccagagagccggcagccccatccccagaaacagagagaaccagagcagaagcagcagccccagagagtga

SEQ ID NO: 29 - protein, 958 aa, Artificial Sequence
  Codon optimized NS3/4A-4Bjunct-Stork HBcAg
MAPITAYAQQTRGLLGCIITSLTGRDKNQVEGEVQIVSTAAQTFLATCINGVCWTVYHGAGTRTIASPKGPVIQMYTNVDQDLVGWPAPQGARSLTPCTCGSSDLYLVTRHADVIPVRRRGDGRGSLLSPRPISYLKGSSGGPLLCPAGHAVGIFRAAVCTRGVAKAVDFIPVESLETTMRSPVFSDNSSPPAVPQSYQVAHLHAPTGSGKSTKVPAAYAAQGYKVLVLNPSVAATMGFGAYMSKAHGIDPNIRTGVRTITTGSPITYSTYGKFLADGGCSGGAYDIIICDECHSTDATSILGIGTVLDQAETAGARLTVLATATPPGSVTVPHPNIEEVALSTTGEIPFYGKAIPLEAIKGGRHLIFCHSKKKCDELAAKLVALGVNAVAYYRGLDVSVIPTSGDVVVVATDALMTGFTGDFDSVIDCNTCVTQTVDFSLDPTFTIETITLPQDAVSRTQRRGRTGRGKPGIYRFVAPGERPSGMFDSSVLCECYDAGCAWYELTPAETTVRLRAYMNTPGLPVCQDHLEFWEGVFTGLTHIDAHFLSQTKQSGENLPYLVAYQATVCARAQAPPPSWDQMWKCLIRLKPTLHGPTPLLYRLGAVQNEVTLTHPVTKYIMTCMSADLEVVTSTWVLVGGVLAALAAYCLSTGCVVIVGRIVLSGKPAIIPDREVLYREFDEMEECSQHLPYIEQGMDVNASRALANVYDLPDDFFPQIDDLVRDAKDALEPYWKAETIKKHVLIATHFVDLIEDFWQTTQGMSQIADALRAVIPPTTTPVPDGYLISHNEAQELPLNDLFVLQEERIVNFQPDYPITARIHTHLRVYTKLNEQALDKARRLLWWHYNCLLWGESNVTNYISRLRTWLSTPEKYRGKDAPTIEAITRPIQVAQGSRNQTKGVRKPRGLEPRRRKVKTTVVYGRRRSKSRGRRSSPSQRAGSPIPRNRENQSRSSSPRE

SEQ ID NO: 30 - DNA, 2877 bp, Artificial Sequence
  Codon optimized NS3/4A-4Bjunct-Stork HBcAg
atggcccccatcaccgcctacgcccagcagacccgcggcctgctgggctgcatcatcaccagcctgaccggccgcgacaagaaccaggtggagggcgaggtgcagatcgtgagcaccgccgcccagaccttcctggccacctgcatcaacggcgtgtgctggaccgtgtaccacggcgccggcacccgcaccatcgccagccccaagggccccgtgatccagatgtacaccaacgtggaccaggacctggtgggctggcccgccccccagggcgcccgcagcctgaccccctgcacctgcggcagcagcgacctgtacctggtgacccgccacgccgacgtgatccccgtgcgccgccgcggcgacggccgcggcagcctgctgagcccccgccccatcagctacctgaagggcagcagcggcggccccctgctgtgccccgccggccacgccgtgggcatcttccgcgccgccgtgtgcacccgcggcgtggccaaggccgtggacttcatccccgtggagagcctggagaccaccatgcgcagccccgtgttcagcgacaacagcagcccccccgccgtgccccagagctaccaggtggcccacctgcacgcccccaccggcagcggcaagagcaccaaggtgcccgccgcctacgccgcccagggctacaaggtgctggtgctgaaccccagcgtggccgccaccatgggcttcggcgcctacatgagcaaggcccacggcatcgaccccaacatccgcaccggcgtgcgcaccatcaccaccggcagccccatcacctacagcacctacggcaagttcctggccgacggcggctgcagcggcggcgcctacgacatcatcatctgcgacgagtgccacagcaccgacgccaccagcatcctgggcatcggcaccgtgctggaccaggccgagaccgccggcgcccgcctgaccgtgctggccaccgccaccccccccggcagcgtgaccgtgccccaccccaacatcgaggaggtggccctgagcaccaccggcgagatccccttctacggcaaggccatccccctggaggccatcaagggcggccgccacctgatcttctgccacagcaagaagaagtgcgacgagctggccgccaagctggtggccctgggcgtgaacgccgtggcctactaccgcggcctggacgtgagcgtgatccccaccagcggcgacgtggtggtggtggccaccgacgccctgatgaccggcttcaccggcgacttcgacagcgtgatcgactgcaacacctgcgtgacccagaccgtggacttcagcctggaccccaccttcaccatcgagaccatcaccctgccccaggacgccgtgagccgcacccagcgccgcggccgcaccggccgcggcaagcccggcatctaccgcttcgtggcccccggcgagcgccccagcggcatgttcgacagcagcgtgctgtgcgagtgctacgacgccggctgcgcctggtacgagctgacccccgccgagaccaccgtgcgcctgcgcgcctacatgaacacccccggcctgcccgtgtgccaggaccacctggagttctgggagggcgtgttcaccggcctgacccacatcgacgcccacttcctgagccagaccaagcagagcggcgagaacctgccctacctggtggcctaccaggccaccgtgtgcgcccgcgcccaggcccccccccccagctgggaccagatgtggaagtgcctgatccgcctgaagcccaccctgcacggccccacccccctgctgtaccgcctgggcgccgtgcagaacgaggtgaccctgacccaccccgtgaccaagtacatcatgacctgcatgagcgccgacctggaggtggtgaccagcacctgggtgctggtgggcggcgtgctggccgccctggccgcctactgcctgagcaccggctgcgtggtgatcgtgggccgcatcgtgctgagcggcaagcccgccatcatccccgaccgcgaggtgctgtaccgcgagttcgacgagatggaggagtgcagccagcacctgccctacatcgagcagggcatggacgtgaacgccagcagagccctggccaacgtgtacgacctgcccgacgacttcttcccccagatcgacgacctggtgagagacgccaaggacgccctggagccctactggaaggccgagaccatcaagaagcacgtgctgatcgccacccacttcgtggacttgatcgaggacttctggcagaccacccagggcatgagccagatcgccgacgccctgagagccgtgatcccccccaccaccacccccgtgcccgacggctacctgatcagccacaacgaggcccaggagctccccctgaacgacctgttcgtgctgcaggaggagagaatcgtgaacttccagcccgactaccccatcaccgccagaattcacacccacctgagagtgtacaccaagctgaacgagcaggccctggacaaggccagaagactgctgtggtggcactacaactgcctgctgtggggcgagagcaacgtgaccaactacatcagcagactgagaacctggctgagcacccccgagaagtacagaggcaaggacgcccccaccatcgaggccatcaccagacccatccaggtggcccagggcagcagaaaccagaccaagggcgtgagaaagcccagaggcctggagcccagaagaagaaaggtgaagaccaccgtggtgtacggcagaagaagaagcaagagcagaggcagaagaagcagccccagccagagagccggcagccccatccccagaaacagagagaaccagagcagaagcagcagccccagagagtga

SEQ ID NO: 31 - protein, 948 aa, Artificial Sequence
  Codon optimized HCV NS3/4A-Heron HBcAg
MAPITAYAQQTRGLLGCIITSLTGRDKNQVEGEVQIVSTAAQTFLATCINGVCWTVYHGAGTRTIASPKGPVIQMYTNVDQDLVGWPAPQGARSLTPCTCGSSDLYLVTRHADVIPVRRRGDGRGSLLSPRPISYLKGSSGGPLLCPAGHAVGIFRAAVCTRGVAKAVDFIPVESLETTMRSPVFSDNSSPPAVPQSYQVAHLHAPTGSGKSTKVPAAYAAQGYKVLVLNPSVAATMGFGAYMSKAHGIDPNIRTGVRTITTGSPITYSTYGKFLADGGCSGGAYDIIICDECHSTDATSILGIGTVLDQAETAGARLTVLATATPPGSVTVPHPNIEEVALSTTGEIPFYGKAIPLEAIKGGRHLIFCHSKKKCDELAAKLVALGVNAVAYYRGLDVSVIPTSGDVVVVATDALMTGFTGDFDSVIDCNTCVTQTVDFSLDPTFTIETITLPQDAVSRTQRRGRTGRGKPGIYRFVAPGERPSGMFDSSVLCECYDAGCAWYELTPAETTVRLRAYMNTPGLPVCQDHLEFWEGVFTGLTHIDAHFLSQTKQSGENLPYLVAYQATVCARAQAPPPSWDQMWKCLIRLKPTLHGPTPLLYRLGAVQNEVTLTHPVTKYIMTCMSADLEVVTPTWVLVGGVLAALAAYCLSTGCVVIVGRIVLSGKPAIIPDREVLYREFDEMEECMDVNASRALANVYDLPDDFFPQIDDLVRDAKDALEPYWKAETIKKHVLIATHFVDLIEDFWQTTQGMSQIADALRAVIPPTTVPVPEGFLITHSEAEELPLNDLFSLQEERIVNFQPDYPITARIHTHLRVYTKLNEQALDKARRLLWWHYNCLLWGEATVTNYISRLRTWLSTPEKYRGKDAPTIEAITRPIQVAQGGRNQTKGTRKPRGLEPRRRKVKTTVVYGRRRSKSRGRRSSPSQRAGSPLPRNRGNQTRSPSPRE

SEQ ID NO: 32 - DNA, 2847 bp, Artificial Sequence
  Codon optimized HCV NS3/4A-Heron HBcAg
atggcccccatcaccgcctacgcccagcagacccgcggcctgctgggctgcatcatcaccagcctgaccggccgcgacaagaaccaggtggagggcgaggtgcagatcgtgagcaccgccgcccagaccttcctggccacctgcatcaacggcgtgtgctggaccgtgtaccacggcgccggcacccgcaccatcgccagccccaagggccccgtgatccagatgtacaccaacgtggaccaggacctggtgggctggcccgccccccagggcgcccgcagcctgaccccctgcacctgcggcagcagcgacctgtacctggtgacccgccacgccgacgtgatccccgtgcgccgccgcggcgacggccgcggcagcctgctgagcccccgccccatcagctacctgaagggcagcagcggcggccccctgctgtgccccgccggccacgccgtgggcatcttccgcgccgccgtgtgcacccgcggcgtggccaaggccgtggacttcatccccgtggagagcctggagaccaccatgcgcagccccgtgttcagcgacaacagcagcccccccgccgtgccccagagctaccaggtggcccacctgcacgcccccaccggcagcggcaagagcaccaaggtgcccgccgcctacgccgcccagggctacaaggtgctggtgctgaaccccagcgtggccgccaccatgggcttcggcgcctacatgagcaaggcccacggcatcgaccccaacatccgcaccggcgtgcgcaccatcaccaccggcagccccatcacctacagcacctacggcaagttcctggccgacggcggctgcagcggcggcgcctacgacatcatcatctgcgacgagtgccacagcaccgacgccaccagcatcctgggcatcggcaccgtgctggaccaggccgagaccgccggcgcccgcctgaccgtgctggccaccgccaccccccccggcagcgtgaccgtgccccaccccaacatcgaggaggtggccctgagcaccaccggcgagatccccttctacggcaaggccatccccctggaggccatcaagggcggccgccacctgatcttctgccacagcaagaagaagtgcgacgagctggccgccaagctggtggccctgggcgtgaacgccgtggcctactaccgcggcctggacgtgagcgtgatccccaccagcggcgacgtggtggtggtggccaccgacgccctgatgaccggcttcaccggcgacttcgacagcgtgatcgactgcaacacctgcgtgacccagaccgtggacttcagcctggaccccaccttcaccatcgagaccatcaccctgccccaggacgccgtgagccgcacccagcgccgcggccgcaccggccgcggcaagcccggcatctaccgcttcgtggcccccggcgagcgccccagcggcatgttcgacagcagcgtgctgtgcgagtgctacgacgccggctgcgcctggtacgagctgacccccgccgagaccaccgtgcgcctgcgcgcctacatgaacacccccggcctgcccgtgtgccaggaccacctggagttctgggagggcgtgttcaccggcctgacccacatcgacgcccacttcctgagccagaccaagcagagcggcgagaacctgccctacctggtggcctaccaggccaccgtgtgcgcccgcgcccaggcccccccccccagctgggaccagatgtggaagtgcctgatccgcctgaagcccaccctgcacggccccacccccctgctgtaccgcctgggcgccgtgcagaacgaggtgaccctgacccaccccgtgaccaagtacatcatgacctgcatgagcgccgacctggaggtggtgacccccacctgggtgctggtgggcggcgtgctggccgccctggccgcctactgcctgagcaccggctgcgtggtgatcgtgggccgcatcgtgctgagcggcaagcccgccatcatccccgaccgcgaggtgctgtaccgcgagttcgacgagatggaggagtgcatggacgtgaacgccagcagagccctggccaacgtgtacgacctgcccgacgacttcttcccccagatcgacgacctggtgagagacgccaaggacgccctggagccctactggaaggccgagaccatcaagaagcacgtgctgatcgccacccacttcgtggacttgatcgaggacttctggcagaccacccagggcatgagccagatcgccgacgccctgagagccgtgatcccccccaccaccgtgcccgtgcccgagggcttcctgatcacccacagcgaggccgaggagctccccctgaacgacctgttcagtctgcaggaggagagaatcgtgaacttccagcccgactaccccatcaccgccagaattcacacccacctgagagtgtacaccaagctgaacgagcaggccctggacaaggccagaagactgctgtggtggcactacaactgcctgctgtggggcgaggccaccgtgaccaactacatcagcagactgagaacctggctgagcacccccgagaagtacagaggcaaggacgcccccaccatcgaggccatcaccagacccatccaggtggcccagggcggcagaaaccagaccaagggcaccagaaagcccagaggcctggagcccagaagaagaaaggtgaagaccaccgtggtgtacggcagaagaagaagcaagagcagaggcagaagaagcagccccagccagagagccggcagccccctgcccagaaacagaggcaaccagaccagaagccccagccccagagagtga

SEQ ID NO: 33 - protein, 948 aa, Artificial Sequence
  Codon optimized mutant NS3/4A-Heron HBcAg
MAPITAYAQQTRGLLGCIITSLTGRDKNQVEGEVQIVSTAAQTFLATCINGVCWTVYAGAGTRTIASPKGPVIQMYTNVDQALVGWPAPQGARSLTPCTCGSSDLYLVTRHADVIPVRRRGDGRGSLLSPRPISYLKGSSGGPLLCPAGHAVGIFRAAVCTRGVAKAVDFIPVESLETTMRSPVFSDNSSPPAVPQSYQVAHLHAPTGSGKSTKVPAAYAAQGYKVLVLNPSVAATMGFGAYMSKAHGIDPNIRTGVRTITTGSPITYSTYGKFLADGGCSGGAYDIIICDECHSTDATSILGIGTVLDQAETAGARLTVLATATPPGSVTVPHPNIEEVALSTTGEIPFYGKAIPLEAIKGGRHLIFCHSKKKCDELAAKLVALGVNAVAYYRGLDVSVIPTSGDVVVVATDALMTGFTGDFDSVIDCNTCVTQTVDFSLDPTFTIETITLPQDAVSRTQRRGRTGRGKPGIYRFVAPGERPSGMFDSSVLCECYDAGCAWYELTPAETTVRLRAYMNTPGLPVCQDHLEFWEGVFTGLTHIDAHFLSQTKQSGENLPYLVAYQATVCARAQAPPPSWDQMWKCLIRLKPTLHGPTPLLYRLGAVQNEVTLTHPVTKYIMTCMSADLEVVTSTWVLVGGVLAALAAYCLSTGCVVIVGRIVLSGKPAIIPDREVLYREFDEMEECMDVNASRALANVYDLPDDFFPQIDDLVRDAKDALEPYWKAETIKKHVLIATHFVDLIEDFWQTTQGMSQIADALRAVIPPTTVPVPEGFLITHSEAEELPLNDLFSLQEERIVNFQPDYPITARIHTHLRVYTKLNEQALDKARRLLWWHYNCLLWGEATVTNYISRLRTWLSTPEKYRGKDAPTIEAITRPIQVAQGGRNQTKGTRKPRGLEPRRRKVKTTVVYGRRRSKSRGRRSSPSQRAGSPLPRNRGNQTRSPSPRE

SEQ ID NO: 34 - DNA, 2847 bp, Artificial Sequence
  Codon optimized mutant NS3/4A-Heron HBcAg
atggcccccatcaccgcctacgcccagcagacccgcggcctgctgggctgcatcatcaccagcctgaccggccgcgacaagaaccaggtggagggcgaggtgcagatcgtgagcaccgccgcccagaccttcctggccacctgcatcaacggcgtgtgctggaccgtgtacgccggcgccggcacccgcaccatcgccagccccaagggccccgtgatccagatgtacaccaacgtggaccaggccctggtgggctggcccgccccccagggcgcccgcagcctgaccccctgcacctgcggcagcagcgacctgtacctggtgacccgccacgccgacgtgatccccgtgcgccgccgcggcgacggccgcggcagcctgctgagcccccgccccatcagctacctgaagggcagcagcggcggccccctgctgtgccccgccggccacgccgtgggcatcttccgcgccgccgtgtgcacccgcggcgtggccaaggccgtggacttcatccccgtggagagcctggagaccaccatgcgcagccccgtgttcagcgacaacagcagcccccccgccgtgccccagagctaccaggtggcccacctgcacgcccccaccggcagcggcaagagcaccaaggtgcccgccgcctacgccgcccagggctacaaggtgctggtgctgaaccccagcgtggccgccaccatgggcttcggcgcctacatgagcaaggcccacggcatcgaccccaacatccgcaccggcgtgcgcaccatcaccaccggcagccccatcacctacagcacctacggcaagttcctggccgacggcggctgcagcggcggcgcctacgacatcatcatctgcgacgagtgccacagcaccgacgccaccagcatcctgggcatcggcaccgtgctggaccaggccgagaccgccggcgcccgcctgaccgtgctggccaccgccaccccccccggcagcgtgaccgtgccccaccccaacatcgaggaggtggccctgagcaccaccggcgagatccccttctacggcaaggccatccccctggaggccatcaagggcggccgccacctgatcttctgccacagcaagaagaagtgcgacgagctggccgccaagctggtggccctgggcgtgaacgccgtggcctactaccgcggcctggacgtgagcgtgatccccaccagcggcgacgtggtggtggtggccaccgacgccctgatgaccggcttcaccggcgacttcgacagcgtgatcgactgcaacacctgcgtgacccagaccgtggacttcagcctggaccccaccttcaccatcgagaccatcaccctgccccaggacgccgtgagccgcacccagcgccgcggccgcaccggccgcggcaagcccggcatctaccgcttcgtggcccccggcgagcgccccagcggcatgttcgacagcagcgtgctgtgcgagtgctacgacgccggctgcgcctggtacgagctgacccccgccgagaccaccgtgcgcctgcgcgcctacatgaacacccccggcctgcccgtgtgccaggaccacctggagttctgggagggcgtgttcaccggcctgacccacatcgacgcccacttcctgagccagaccaagcagagcggcgagaacctgccctacctggtggcctaccaggccaccgtgtgcgcccgcgcccaggcccccccccccagctgggaccagatgtggaagtgcctgatccgcctgaagcccaccctgcacggccccacccccctgctgtaccgcctgggcgccgtgcagaacgaggtgaccctgacccaccccgtgaccaagtacatcatgacctgcatgagcgccgacctggaggtggtgaccagcacctgggtgctggtgggcggcgtgctggccgccctggccgcctactgcctgagcaccggctgcgtggtgatcgtgggccgcatcgtgctgagcggcaagcccgccatcatccccgaccgcgaggtgctgtaccgcgagttcgacgagatggaggagtgcatggacgtgaacgccagcagagccctggccaacgtgtacgacctgcccgacgacttcttcccccagatcgacgacctggtgagagacgccaaggacgccctggagccctactggaaggccgagaccatcaagaagcacgtgctgatcgccacccacttcgtggacttgatcgaggacttctggcagaccacccagggcatgagccagatcgccgacgccctgagagccgtgatcccccccaccaccgtgcccgtgcccgagggcttcctgatcacccacagcgaggccgaggagctccccctgaacgacctgttcagtctgcaggaggagagaatcgtgaacttccagcccgactaccccatcaccgccagaattcacacccacctgagagtgtacaccaagctgaacgagcaggccctggacaaggccagaagactgctgtggtggcactacaactgcctgctgtggggcgaggccaccgtgaccaactacatcagcagactgagaacctggctgagcacccccgagaagtacagaggcaaggacgcccccaccatcgaggccatcaccagacccatccaggtggcccagggcggcagaaaccagaccaagggcaccagaaagcccagaggcctggagcccagaagaagaaaggtgaagaccaccgtggtgtacggcagaagaagaagcaagagcagaggcagaagaagcagccccagccagagagccggcagccccctgcccagaaacagaggcaaccagaccagaagccccagccccagagagtga

SEQ ID NO: 35 - protein, 948 aa, Artificial Sequence
  Codon optimized NS3/4A-Heron HBcAg
MAPITAYAQQTRGLLGCIITSLTGRDKNQVEGEVQIVSTAAQTFLATCINGVCWTVYHGAGTRTIASPKGPVIQMYTNVDQDLVGWPAPQGARSLTPCTCGSSDLYLVTRHADVIPVRRRGDGRGSLLSPRPISYLKGSSGGPLLCPAGHAVGIFRAAVCTRGVAKAVDFIPVESLETTMRSPVFSDNSSPPAVPQSYQVAHLHAPTGSGKSTKVPAAYAAQGYKVLVLNPSVAATMGFGAYMSKAHGIDPNIRTGVRTITTGSPITYSTYGKFLADGGCSGGAYDIIICDECHSTDATSILGIGTVLDQAETAGARLTVLATATPPGSVTVPHPNIEEVALSTTGEIPFYGKAIPLEAIKGGRHLIFCHSKKKCDELAAKLVALGVNAVAYYRGLDVSVIPTSGDVVVVATDALMTGFTGDFDSVIDCNTCVTQTVDFSLDPTFTIETITLPQDAVSRTQRRGRTGRGKPGIYRFVAPGERPSGMFDSSVLCECYDAGCAWYELTPAETTVRLRAYMNTPGLPVCQDHLEFWEGVFTGLTHIDAHFLSQTKQSGENLPYLVAYQATVCARAQAPPPSWDQMWKCLIRLKPTLHGPTPLLYRLGAVQNEVTLTHPVTKYIMTCMSADLEVVTSTWVLVGGVLAALAAYCLSTGCVVIVGRIVLSGKPAIIPDREVLYREFDEMEECMDVNASRALANVYDLPDDFFPQIDDLVRDAKDALEPYWKAETIKKHVLIATHFVDLIEDFWQTTQGMSQIADALRAVIPPTTVPVPEGFLITHSEAEELPLNDLFSLQEERIVNFQPDYPITARIHTHLRVYTKLNEQALDKARRLLWWHYNCLLWGEATVTNYISRLRTWLSTPEKYRGKDAPTIEAITRPIQVAQGGRNQTKGTRKPRGLEPRRRKVKTTVVYGRRRSKSRGRRSSPSQRAGSPLPRNRGNQTRSPSPRE

SEQ ID NO: 36 - DNA, 2847 bp, Artificial Sequence
  Codon optimized NS3/4A-Heron HBcAg
atggcccccatcaccgcctacgcccagcagacccgcggcctgctgggctgcatcatcaccagcctgaccggccgcgacaagaaccaggtggagggcgaggtgcagatcgtgagcaccgccgcccagaccttcctggccacctgcatcaacggcgtgtgctggaccgtgtaccacggcgccggcacccgcaccatcgccagccccaagggccccgtgatccagatgtacaccaacgtggaccaggacctggtgggctggcccgccccccagggcgcccgcagcctgaccccctgcacctgcggcagcagcgacctgtacctggtgacccgccacgccgacgtgatccccgtgcgccgccgcggcgacggccgcggcagcctgctgagcccccgccccatcagctacctgaagggcagcagcggcggccccctgctgtgccccgccggccacgccgtgggcatcttccgcgccgccgtgtgcacccgcggcgtggccaaggccgtggacttcatccccgtggagagcctggagaccaccatgcgcagccccgtgttcagcgacaacagcagcccccccgccgtgccccagagctaccaggtggcccacctgcacgcccccaccggcagcggcaagagcaccaaggtgcccgccgcctacgccgcccagggctacaaggtgctggtgctgaaccccagcgtggccgccaccatgggcttcggcgcctacatgagcaaggcccacggcatcgaccccaacatccgcaccggcgtgcgcaccatcaccaccggcagccccatcacctacagcacctacggcaagttcctggccgacggcggctgcagcggcggcgcctacgacatcatcatctgcgacgagtgccacagcaccgacgccaccagcatcctgggcatcggcaccgtgctggaccaggccgagaccgccggcgcccgcctgaccgtgctggccaccgccaccccccccggcagcgtgaccgtgccccaccccaacatcgaggaggtggccctgagcaccaccggcgagatccccttctacggcaaggccatccccctggaggccatcaagggcggccgccacctgatcttctgccacagcaagaagaagtgcgacgagctggccgccaagctggtggccctgggcgtgaacgccgtggcctactaccgcggcctggacgtgagcgtgatccccaccagcggcgacgtggtggtggtggccaccgacgccctgatgaccggcttcaccggcgacttcgacagcgtgatcgactgcaacacctgcgtgacccagaccgtggacttcagcctggaccccaccttcaccatcgagaccatcaccctgccccaggacgccgtgagccgcacccagcgccgcggccgcaccggccgcggcaagcccggcatctaccgcttcgtggcccccggcgagcgccccagcggcatgttcgacagcagcgtgctgtgcgagtgctacgacgccggctgcgcctggtacgagctgacccccgccgagaccaccgtgcgcctgcgcgcctacatgaacacccccggcctgcccgtgtgccaggaccacctggagttctgggagggcgtgttcaccggcctgacccacatcgacgcccacttcctgagccagaccaagcagagcggcgagaacctgccctacctggtggcctaccaggccaccgtgtgcgcccgcgcccaggcccccccccccagctgggaccagatgtggaagtgcctgatccgcctgaagcccaccctgcacggccccacccccctgctgtaccgcctgggcgccgtgcagaacgaggtgaccctgacccaccccgtgaccaagtacatcatgacctgcatgagcgccgacctggaggtggtgaccagcacctgggtgctggtgggcggcgtgctggccgccctggccgcctactgcctgagcaccggctgcgtggtgatcgtgggccgcatcgtgctgagcggcaagcccgccatcatccccgaccgcgaggtgctgtaccgcgagttcgacgagatggaggagtgcatggacgtgaacgccagcagagccctggccaacgtgtacgacctgcccgacgacttcttcccccagatcgacgacctggtgagagacgccaaggacgccctggagccctactggaaggccgagaccatcaagaagcacgtgctgatcgccacccacttcgtggacttgatcgaggacttctggcagaccacccagggcatgagccagatcgccgacgccctgagagccgtgatcccccccaccaccgtgcccgtgcccgagggcttcctgatcacccacagcgaggccgaggagctccccctgaacgacctgttcagtctgcaggaggagagaatcgtgaacttccagcccgactaccccatcaccgccagaattcacacccacctgagagtgtacaccaagctgaacgagcaggccctggacaaggccagaagactgctgtggtggcactacaactgcctgctgtggggcgaggccaccgtgaccaactacatcagcagactgagaacctggctgagcacccccgagaagtacagaggcaaggacgcccccaccatcgaggccatcaccagacccatccaggtggcccagggcggcagaaaccagaccaagggcaccagaaagcccagaggcctggagcccagaagaagaaaggtgaagaccaccgtggtgtacggcagaagaagaagcaagagcagaggcagaagaagcagccccagccagagagccggcagccccctgcccagaaacagaggcaaccagaccagaagccccagccccagagagtga

SEQ ID NO: 37 - protein, 958 aa, Artificial Sequence
  Codon optimized NS3/4A-4B junct-Heron HBcAg
MAPITAYAQQTRGLLGCIITSLTGRDKNQVEGEVQIVSTAAQTFLATCINGVCWTVYHGAGTRTIASPKGPVIQMYTNVDQDLVGWPAPQGARSLTPCTCGSSDLYLVTRHADVIPVRRRGDGRGSLLSPRPISYLKGSSGGPLLCPAGHAVGIFRAAVCTRGVAKAVDFIPVESLETTMRSPVFSDNSSPPAVPQSYQVAHLHAPTGSGKSTKVPAAYAAQGYKVLVLNPSVAATMGFGAYMSKAHGIDPNIRTGVRTITTGSPITYSTYGKFLADGGCSGGAYDIIICDECHSTDATSILGIGTVLDQAETAGARLTVLATATPPGSVTVPHPNIEEVALSTTGEIPFYGKAIPLEAIKGGRHLIFCHSKKKCDELAAKLVALGVNAVAYYRGLDVSVIPTSGDVVVVATDALMTGFTGDFDSVIDCNTCVTQTVDFSLDPTFTIETITLPQDAVSRTQRRGRTGRGKPGIYRFVAPGERPSGMFDSSVLCECYDAGCAWYELTPAETTVRLRAYMNTPGLPVCQDHLEFWEGVFTGLTHIDAHFLSQTKQSGENLPYLVAYQATVCARAQAPPPSWDQMWKCLIRLKPTLHGPTPLLYRLGAVQNEVTLTHPVTKYIMTCMSADLEVVTSTWVLVGGVLAALAAYCLSTGCVVIVGRIVLSGKPAIIPDREVLYREFDEMEECSQHLPYIEQGMDVNASRALANVYDLPDDFFPQIDDLVRDAKDALEPYWKAETIKKHVLIATHFVDLIEDFWQTTQGMSQIADALRAVIPPTTVPVPEGFLITHSEAEELPLNDLFSLQEERIVNFQPDYPITARIHTHLRVYTKLNEQALDKARRLLWWHYNCLLWGEATVTNYISRLRTWLSTPEKYRGKDAPTIEAITRPIQVAQGGRNQTKGTRKPRGLEPRRRKVKTTVVYGRRRSKSRGRRSSPSQRAGSPLPRNRGNQTRSPSPRE

SEQ ID NO: 38 - DNA, 2877 bp, Artificial Sequence
  Codon optimized NS3/4A-4B junct-Heron HBcAg
atggcccccatcaccgcctacgcccagcagacccgcggcctgctgggctgcatcatcaccagcctgaccggccgcgacaagaaccaggtggagggcgaggtgcagatcgtgagcaccgccgcccagaccttcctggccacctgcatcaacggcgtgtgctggaccgtgtaccacggcgccggcacccgcaccatcgccagccccaagggccccgtgatccagatgtacaccaacgtggaccaggacctggtgggctggcccgccccccagggcgcccgcagcctgaccccctgcacctgcggcagcagcgacctgtacctggtgacccgccacgccgacgtgatccccgtgcgccgccgcggcgacggccgcggcagcctgctgagcccccgccccatcagctacctgaagggcagcagcggcggccccctgctgtgccccgccggccacgccgtgggcatcttccgcgccgccgtgtgcacccgcggcgtggccaaggccgtggacttcatccccgtggagagcctggagaccaccatgcgcagccccgtgttcagcgacaacagcagcccccccgccgtgccccagagctaccaggtggcccacctgcacgcccccaccggcagcggcaagagcaccaaggtgcccgccgcctacgccgcccagggctacaaggtgctggtgctgaaccccagcgtggccgccaccatgggcttcggcgcctacatgagcaaggcccacggcatcgaccccaacatccgcaccggcgtgcgcaccatcaccaccggcagccccatcacctacagcacctacggcaagttcctggccgacggcggctgcagcggcggcgcctacgacatcatcatctgcgacgagtgccacagcaccgacgccaccagcatcctgggcatcggcaccgtgctggaccaggccgagaccgccggcgcccgcctgaccgtgctggccaccgccaccccccccggcagcgtgaccgtgccccaccccaacatcgaggaggtggccctgagcaccaccggcgagatccccttctacggcaaggccatccccctggaggccatcaagggcggccgccacctgatcttctgccacagcaagaagaagtgcgacgagctggccgccaagctggtggccctgggcgtgaacgccgtggcctactaccgcggcctggacgtgagcgtgatccccaccagcggcgacgtggtggtggtggccaccgacgccctgatgaccggcttcaccggcgacttcgacagcgtgatcgactgcaacacctgcgtgacccagaccgtggacttcagcctggaccccaccttcaccatcgagaccatcaccctgccccaggacgccgtgagccgcacccagcgccgcggccgcaccggccgcggcaagcccggcatctaccgcttcgtggcccccggcgagcgccccagcggcatgttcgacagcagcgtgctgtgcgagtgctacgacgccggctgcgcctggtacgagctgacccccgccgagaccaccgtgcgcctgcgcgcctacatgaacacccccggcctgcccgtgtgccaggaccacctggagttctgggagggcgtgttcaccggcctgacccacatcgacgcccacttcctgagccagaccaagcagagcggcgagaacctgccctacctggtggcctaccaggccaccgtgtgcgcccgcgcccaggcccccccccccagctgggaccagatgtggaagtgcctgatccgcctgaagcccaccctgcacggccccacccccctgctgtaccgcctgggcgccgtgcagaacgaggtgaccctgacccaccccgtgaccaagtacatcatgacctgcatgagcgccgacctggaggtggtgaccagcacctgggtgctggtgggcggcgtgctggccgccctggccgcctactgcctgagcaccggctgcgtggtgatcgtgggccgcatcgtgctgagcggcaagcccgccatcatccccgaccgcgaggtgctgtaccgcgagttcgacgagatggaggagtgcagccagcacctgccctacatcgagcagggcatggacgtgaacgccagcagagccctggccaacgtgtacgacctgcccgacgacttcttcccccagatcgacgacctggtgagagacgccaaggacgccctggagccctactggaaggccgagaccatcaagaagcacgtgctgatcgccacccacttcgtggacttgatcgaggacttctggcagaccacccagggcatgagccagatcgccgacgccctgagagccgtgatcccccccaccaccgtgcccgtgcccgagggcttcctgatcacccacagcgaggccgaggagctccccctgaacgacctgttcagtctgcaggaggagagaatcgtgaacttccagcccgactaccccatcaccgccagaattcacacccacctgagagtgtacaccaagctgaacgagcaggccctggacaaggccagaagactgctgtggtggcactacaactgcctgctgtggggcgaggccaccgtgaccaactacatcagcagactgagaacctggctgagcacccccgagaagtacagaggcaaggacgcccccaccatcgaggccatcaccagacccatccaggtggcccagggcggcagaaaccagaccaagggcaccagaaagcccagaggcctggagcccagaagaagaaaggtgaagaccaccgtggtgtacggcagaagaagaagcaagagcagaggcagaagaagcagccccagccagagagccggcagccccctgcccagaaacagaggcaaccagaccagaagccccagccccagagagtga

SEQ ID NO: 39 - protein, 711 aa, Artificial Sequence
  Codon optimized HCV NS5A-Stork HBcAg
MGSGSWLRDVWDWICTVLTDFKTWLQSKLLPKLPGVPFFSCQRGYKGVWRGDGIMQTTCPCGAQITGHVKNGSMRIVGPKTCSNTWHGTFPINAYTTGPCTPSPAPNYSRALWRVAAEEYVEITRVGDFHYVTGMTTDNVKCPCQVPAPEFFTELDGVRLHRYAPACRPLLREDVTFQVGLNQYLVGSQLPCEPEPDVAVLTSMLTDPSHITAETAKRRLARGSPPSLASSSASQLSAPSLKATCTTHHDSPDADLIEANLLWRQEMGGNITRVESENKVVILDSFDPLRAEEDEREVSVAAEILRKSKKFPPALPIWARPDYNPPLLESWKSPDYVPPAVHGCPLPPTTGPPIPPPRKKRTVVLTESTVSSALAELATKTFGSSGSSAVDSGTATAPPDQTSDDGDKESDIESYSSMPPLEGEPGDPDLSDGSWSTVSGEAGDDIVCCMDVNASRALANVYDLPDDFFPQIDDLVRDAKDALEPYWKAETIKKHVLIATHFVDLIEDFWQTTQGMSQIADALRAVIPPTTTPVPDGYLISHNEAQELPLNDLFVLQEERIVNFQPDYPITARIHTHLRVYTKLNEQALDKARRLLWWHYNCLLWGESNVTNYISRLRTWLSTPEKYRGKDAPTIEAITRPIQVAQGSRNQTKGVRKPRGLEPRRRKVKTTVVYGRRRSKSRGRRSSPSQRAGSPIPRNRENQSRSSSPRE

SEQ ID NO: 40 - DNA, 2136 bp, Artificial Sequence
  Codon optimized HCV NS5A-Stork HBcAg
atgggatctggaagctggctgagggatgtttgggattggatttgtaccgtcctcaccgacttcaaaacctggctccagtccaagctgctgccaaagctgcccggagtgccattcttctcctgtcagaggggctataaaggcgtgtggaggggcgatggaattatgcagactacttgcccctgtggagctcaaattactgggcacgttaagaatggctccatgcggattgttggcccaaaaacctgttccaacacctggcacggaaccttccctattaacgcttacaccaccggaccttgcactccttcccccgcacctaattattcccgggctctctggcgggtggcagcagaggaatatgtcgaaattaccagagtcggcgacttccactacgtcacaggaatgactacagacaacgttaaatgtccctgccaagtgcccgctccagagttctttaccgaactcgacggggttaggctccacagatacgcacccgcctgccggccactgctgcgggaagacgtcacattccaggtcgggctgaaccagtacctggtgggctctcagctgccttgtgagcctgagcccgacgtggcagttctcaccagcatgctcaccgatcctagccacatcaccgctgagacagccaaacgccgcctggctagagggtcccctccctctctggccagctccagcgctagccagctctccgcaccaagcctgaaagccacatgcactacacaccacgatagccccgacgcagacctgattgaagccaacctcctctggagacaggaaatgggcggaaacatcactagggtcgaatccgagaataaagtggttattctggatagcttcgacccactcagggcagaggaagatgagagagaggttagcgtggccgctgagattctccgcaagtccaaaaagttccctcccgcactgcccatttgggcaaggcccgattacaatcctccactgctcgagagctggaagtcccctgactacgtgccaccagccgtccacggatgccctctgccccctaccacaggaccaccaattccaccccctagaaagaaacggaccgtggttctgactgagtccaccgtgtcctctgcactcgctgagctggcaaccaagacctttggatccagcggatcctccgcagtcgactccggcaccgctaccgccccacccgatcaaacctctgacgatggagacaaggagagcgatattgagtcctattccagcatgcccccactcgagggagaacccggcgaccccgacctgagcgatgggtcctggagcactgtgagcggggaagcaggggacgacattgtctgttgcatggacgtgaacgccagcagagccctggccaacgtgtacgacctgcccgacgacttcttcccccagatcgacgacctggtgagagacgccaaggacgccctggagccctactggaaggccgagaccatcaagaagcacgtgctgatcgccacccacttcgtggacttgatcgaggacttctggcagaccacccagggcatgagccagatcgccgacgccctgagagccgtgatcccccccaccaccacccccgtgcccgacggctacctgatcagccacaacgaggcccaggagctccccctgaacgacctgttcgtgctgcaggaggagagaatcgtgaacttccagcccgactaccccatcaccgccagaattcacacccacctgagagtgtacaccaagctgaacgagcaggccctggacaaggccagaagactgctgtggtggcactacaactgcctgctgtggggcgagagcaacgtgaccaactacatcagcagactgagaacctggctgagcacccccgagaagtacagaggcaaggacgcccccaccatcgaggccatcaccagacccatccaggtggcccagggcagcagaaaccagaccaagggcgtgagaaagcccagaggcctggagcccagaagaagaaaggtgaagaccaccgtggtgtacggcagaagaagaagcaagagcagaggcagaagaagcagccccagccagagagccggcagccccatccccagaaacagagagaaccagagcagaagcagcagccccagagagtga

SEQ ID NO: 41 - protein, 711 aa, Artificial Sequence
  Codon optimized HCV NS5A-Heron HBcAg
MGSGSWLRDVWDWICTVLTDFKTWLQSKLLPKLPGVPFFSCQRGYKGVWRGDGIMQTTCPCGAQITGHVKNGSMRIVGPKTCSNTWHGTFPINAYTTGPCTPSPAPNYSRALWRVAAEEYVEITRVGDFHYVTGMTTDNVKCPCQVPAPEFFTELDGVRLHRYAPACRPLLREDVTFQVGLNQYLVGSQLPCEPEPDVAVLTSMLTDPSHITAETAKRRLARGSPPSLASSSASQLSAPSLKATCTTHHDSPDADLIEANLLWRQEMGGNITRVESENKVVILDSFDPLRAEEDEREVSVAAEILRKSKKFPPALPIWARPDYNPPLLESWKSPDYVPPAVHGCPLPPTTGPPIPPPRKKRTVVLTESTVSSALAELATKTFGSSGSSAVDSGTATAPPDQTSDDGDKESDIESYSSMPPLEGEPGDPDLSDGSWSTVSGEAGDDIVCCMDVNASRALANVYDLPDDFFPQIDDLVRDAKDALEPYWKAETIKKHVLIATHFVDLIEDFWQTTQGMSQIADALRAVIPPTTVPVPEGFLITHSEAEELPLNDLFSLQEERIVNFQPDYPITARIHTHLRVYTKLNEQALDKARRLLWWHYNCLLWGEATVTNYISRLRTWLSTPEKYRGKDAPTIEAITRPIQVAQGGRNQTKGTRKPRGLEPRRRKVKTTVVYGRRRSKSRGRRSSPSQRAGSPLPRNRGNQTRSPSPRE

SEQ ID NO: 42 - DNA, 2136 bp, Artificial Sequence
  Codon optimized HCV NS5A-Heron HBcAg
atgggatctggaagctggctgagggatgtttgggattggatttgtaccgtcctcaccgacttcaaaacctggctccagtccaagctgctgccaaagctgcccggagtgccattcttctcctgtcagaggggctataaaggcgtgtggaggggcgatggaattatgcagactacttgcccctgtggagctcaaattactgggcacgttaagaatggctccatgcggattgttggcccaaaaacctgttccaacacctggcacggaaccttccctattaacgcttacaccaccggaccttgcactccttcccccgcacctaattattcccgggctctctggcgggtggcagcagaggaatatgtcgaaattaccagagtcggcgacttccactacgtcacaggaatgactacagacaacgttaaatgtccctgccaagtgcccgctccagagttctttaccgaactcgacggggttaggctccacagatacgcacccgcctgccggccactgctgcgggaagacgtcacattccaggtcgggctgaaccagtacctggtgggctctcagctgccttgtgagcctgagcccgacgtggcagttctcaccagcatgctcaccgatcctagccacatcaccgctgagacagccaaacgccgcctggctagagggtcccctccctctctggccagctccagcgctagccagctctccgcaccaagcctgaaagccacatgcactacacaccacgatagccccgacgcagacctgattgaagccaacctcctctggagacaggaaatgggcggaaacatcactagggtcgaatccgagaataaagtggttattctggatagcttcgacccactcagggcagaggaagatgagagagaggttagcgtggccgctgagattctccgcaagtccaaaaagttccctcccgcactgcccatttgggcaaggcccgattacaatcctccactgctcgagagctggaagtcccctgactacgtgccaccagccgtccacggatgccctctgccccctaccacaggaccaccaattccaccccctagaaagaaacggaccgtggttctgactgagtccaccgtgtcctctgcactcgctgagctggcaaccaagacctttggatccagcggatcctccgcagtcgactccggcaccgctaccgccccacccgatcaaacctctgacgatggagacaaggagagcgatattgagtcctattccagcatgcccccactcgagggagaacccggcgaccccgacctgagcgatgggtcctggagcactgtgagcggggaagcaggggacgacattgtctgttgcatggacgtgaacgccagcagagccctggccaacgtgtacgacctgcccgacgacttcttcccccagatcgacgacctggtgagagacgccaaggacgccctggagccctactggaaggccgagaccatcaagaagcacgtgctgatcgccacccacttcgtggacttgatcgaggacttctggcagaccacccagggcatgagccagatcgccgacgccctgagagccgtgatcccccccaccaccgtgcccgtgcccgagggcttcctgatcacccacagcgaggccgaggagctccccctgaacgacctgttcagtctgcaggaggagagaatcgtgaacttccagcccgactaccccatcaccgccagaattcacacccacctgagagtgtacaccaagctgaacgagcaggccctggacaaggccagaagactgctgtggtggcactacaactgcctgctgtggggcgaggccaccgtgaccaactacatcagcagactgagaacctggctgagcacccccgagaagtacagaggcaaggacgcccccaccatcgaggccatcaccagacccatccaggtggcccagggcggcagaaaccagaccaagggcaccagaaagcccagaggcctggagcccagaagaagaaaggtgaagaccaccgtggtgtacggcagaagaagaagcaagagcagaggcagaagaagcagccccagccagagagccggcagccccctgcccagaaacagaggcaaccagaccagaagccccagccccagagagtga

SEQ ID NO: 43 - protein, 474 aa, Artificial Sequence
  Codon optimized HBV HBeAg-Stork HBcAg
MQLFHLCLIISCSCPTVQASKLCLGWLWGMDIDPYKEFGATAELLSFLPSDFFPSVRDLLDTASALYREALESPEHCSPHHTALRQAILCWGELMTLATWVGVNLEDPASRDLVVSYVNTNMGLKFRQLLWFHISCLTFGRETVIEYLVSFGVWIRTPPAYRPPNAPILSTLPETTVVRRRGRSPRRRTPSPRRRRSQSPRRRRSQSRESQCMDVNASRALANVYDLPDDFFPQIDDLVRDAKDALEPYWKAETIKKHVLIATHFVDLIEDFWQTTQGMSQIADALRAVIPPTTTPVPDGYLISHNEAQELPLNDLFVLQEERIVNFQPDYPITARIHTHLRVYTKLNEQALDKARRLLWWHYNCLLWGESNVTNYISRLRTWLSTPEKYRGKDAPTIEAITRPIQVAQGSRNQTKGVRKPRGLEPRRRKVKTTVVYGRRRSKSRGRRSSPSQRAGSPIPRNRENQSRSSSPRE

SEQ ID NO: 44 - DNA, 1425 bp, Artificial Sequence
  Codon optimized HBV HBeAg-Stork HBcAg
atgcagctgttccacctgtgcctgatcatcagctgcagctgccccaccgtgcaggccagcaagctgtgcctgggctggctgtggggcatggacatcgacccctacaaggagttcggcgccaccgccgagctgctgagcttcctgcccagcgacttcttccccagcgtgagagacctgctggacaccgccagcgccctgtacagagaggccctggagagccccgagcactgcagcccccaccacaccgccctgagacaggccatcctgtgctggggcgagctgatgaccctggccacctgggtgggcgtgaacctggaggaccccgccagcagagacctggtggtgagctacgtgaacaccaacatgggcctgaagttcagacagctgctgtggttccacatcagctgcctgaccttcggcagagagaccgtgatcgagtacctggtgagcttcggcgtgtggatcagaaccccccccgcctacagaccccccaacgcccccatcctgagcaccctgcccgagaccaccgtggtgagaagaagaggcagaagccccagaagaagaacccccagccccagaagaagaagaagccagagccccagaagaagaagaagccagagcagagagagccagtgcatggacgtgaacgccagcagagccctggccaacgtgtacgacctgcccgacgacttcttcccccagatcgacgacctggtgagagacgccaaggacgccctggagccctactggaaggccgagaccatcaagaagcacgtgctgatcgccacccacttcgtggacttgatcgaggacttctggcagaccacccagggcatgagccagatcgccgacgccctgagagccgtgatcccccccaccaccacccccgtgcccgacggctacctgatcagccacaacgaggcccaggagctccccctgaacgacctgttcgtgctgcaggaggagagaatcgtgaacttccagcccgactaccccatcaccgccagaattcacacccacctgagagtgtacaccaagctgaacgagcaggccctggacaaggccagaagactgctgtggtggcactacaactgcctgctgtggggcgagagcaacgtgaccaactacatcagcagactgagaacctggctgagcacccccgagaagtacagaggcaaggacgcccccaccatcgaggccatcaccagacccatccaggtggcccagggcagcagaaaccagaccaagggcgtgagaaagcccagaggcctggagcccagaagaagaaaggtgaagaccaccgtggtgtacggcagaagaagaagcaagagcagaggcagaagaagcagccccagccagagagccggcagccccatccccagaaacagagagaaccagagcagaagcagcagccccagagagtga

SEQ ID NO: 45 - protein, 474 aa, Artificial Sequence
  Codon optimized HBV HBeAg-Stork HBcAg
MQLFHLCLIISCSCPTVQASKLCLGWLWGLDIDPYKEFGATAELLSFLPSDFFPSVRDLLDTASALYREALESPEHCSPHHTALRQAILCWGELMTLATWVGVNLEDPASRDLVVSYVNTNMGLKFRQLLWFHISCLTFGRETVIEYLVSFGVWIRTPPAYRPPNAPILSTLPETTVVRRRGRSPRRRTPSPRRRRSQSPRRRRSQSRESQCMDVNASRALANVYDLPDDFFPQIDDLVRDAKDALEPYWKAETIKKHVLIATHFVDLIEDFWQTTQGMSQIADALRAVIPPTTTPVPDGYLISHNEAQELPLNDLFVLQEERIVNFQPDYPITARIHTHLRVYTKLNEQALDKARRLLWWHYNCLLWGESNVTNYISRLRTWLSTPEKYRGKDAPTIEAITRPIQVAQGSRNQTKGVRKPRGLEPRRRKVKTTVVYGRRRSKSRGRRSSPSQRAGSPIPRNRENQSRSSSPRE

SEQ ID NO: 46 - DNA, 1425 bp, Artificial Sequence
  Codon optimized HBV HBeAg-Stork HBcAg
atgcagctgttccacctgtgcctgatcatcagctgcagctgccccaccgtgcaggccagcaagctgtgcctgggctggctgtggggcctggacatcgacccctacaaggagttcggcgccaccgccgagctgctgagcttcctgcccagcgacttcttccccagcgtgagagacctgctggacaccgccagcgccctgtacagagaggccctggagagccccgagcactgcagcccccaccacaccgccctgagacaggccatcctgtgctggggcgagctgatgaccctggccacctgggtgggcgtgaacctggaggaccccgccagcagagacctggtggtgagctacgtgaacaccaacatgggcctgaagttcagacagctgctgtggttccacatcagctgcctgaccttcggcagagagaccgtgatcgagtacctggtgagcttcggcgtgtggatcagaaccccccccgcctacagaccccccaacgcccccatcctgagcaccctgcccgagaccaccgtggtgagaagaagaggcagaagccccagaagaagaacccccagccccagaagaagaagaagccagagccccagaagaagaagaagccagagcagagagagccagtgcatggacgtgaacgccagcagagccctggccaacgtgtacgacctgcccgacgacttcttcccccagatcgacgacctggtgagagacgccaaggacgccctggagccctactggaaggccgagaccatcaagaagcacgtgctgatcgccacccacttcgtggacttgatcgaggacttctggcagaccacccagggcatgagccagatcgccgacgccctgagagccgtgatcccccccaccaccacccccgtgcccgacggctacctgatcagccacaacgaggcccaggagctccccctgaacgacctgttcgtgctgcaggaggagagaatcgtgaacttccagcccgactaccccatcaccgccagaattcacacccacctgagagtgtacaccaagctgaacgagcaggccctggacaaggccagaagactgctgtggtggcactacaactgcctgctgtggggcgagagcaacgtgaccaactacatcagcagactgagaacctggctgagcacccccgagaagtacagaggcaaggacgcccccaccatcgaggccatcaccagacccatccaggtggcccagggcagcagaaaccagaccaagggcgtgagaaagcccagaggcctggagcccagaagaagaaaggtgaagaccaccgtggtgtacggcagaagaagaagcaagagcagaggcagaagaagcagccccagccagagagccggcagccccatccccagaaacagagagaaccagagcagaagcagcagccccagagagtga

SEQ ID NO: 47 - protein, 474 aa, Artificial Sequence
  Codon optimized HBV HBeAg-Heron HBcAg
MQLFHLCLIISCSCPTVQASKLCLGWLWGMDIDPYKEFGATAELLSFLPSDFFPSVRDLLDTASALYREALESPEHCSPHHTALRQAILCWGELMTLATWVGVNLEDPASRDLVVSYVNTNMGLKFRQLLWFHISCLTFGRETVIEYLVSFGVWIRTPPAYRPPNAPILSTLPETTVVRRRGRSPRRRTPSPRRRRSQSPRRRRSQSRESQCMDVNASRALANVYDLPDDFFPQIDDLVRDAKDALEPYWKAETIKKHVLIATHFVDLIEDFWQTTQGMSQIADALRAVIPPTTVPVPEGFLITHSEAEELPLNDLFSLQEERIVNFQPDYPITARIHTHLRVYTKLNEQALDKARRLLWWHYNCLLWGEATVTNYISRLRTWLSTPEKYRGKDAPTIEAITRPIQVAQGGRNQTKGTRKPRGLEPRRRKVKTTVVYGRRRSKSRGRRSSPSQRAGSPLPRNRGNQTRSPSPRE

SEQ ID NO: 48 - DNA, 1425 bp, Artificial Sequence
  Codon optimized HBV HBeAg-Heron HBcAg
atgcagctgttccacctgtgcctgatcatcagctgcagctgccccaccgtgcaggccagcaagctgtgcctgggctggctgtggggcatggacatcgacccctacaaggagttcggcgccaccgccgagctgctgagcttcctgcccagcgacttcttccccagcgtgagagacctgctggacaccgccagcgccctgtacagagaggccctggagagccccgagcactgcagcccccaccacaccgccctgagacaggccatcctgtgctggggcgagctgatgaccctggccacctgggtgggcgtgaacctggaggaccccgccagcagagacctggtggtgagctacgtgaacaccaacatgggcctgaagttcagacagctgctgtggttccacatcagctgcctgaccttcggcagagagaccgtgatcgagtacctggtgagcttcggcgtgtggatcagaaccccccccgcctacagaccccccaacgcccccatcctgagcaccctgcccgagaccaccgtggtgagaagaagaggcagaagccccagaagaagaacccccagccccagaagaagaagaagccagagccccagaagaagaagaagccagagcagagagagccagtgcatggacgtgaacgccagcagagccctggccaacgtgtacgacctgcccgacgacttcttcccccagatcgacgacctggtgagagacgccaaggacgccctggagccctactggaaggccgagaccatcaagaagcacgtgctgatcgccacccacttcgtggacttgatcgaggacttctggcagaccacccagggcatgagccagatcgccgacgccctgagagccgtgatcccccccaccaccgtgcccgtgcccgagggcttcctgatcacccacagcgaggccgaggagctccccctgaacgacctgttcagtctgcaggaggagagaatcgtgaacttccagcccgactaccccatcaccgccagaattcacacccacctgagagtgtacaccaagctgaacgagcaggccctggacaaggccagaagactgctgtggtggcactacaactgcctgctgtggggcgaggccaccgtgaccaactacatcagcagactgagaacctggctgagcacccccgagaagtacagaggcaaggacgcccccaccatcgaggccatcaccagacccatccaggtggcccagggcggcagaaaccagaccaagggcaccagaaagcccagaggcctggagcccagaagaagaaaggtgaagaccaccgtggtgtacggcagaagaagaagcaagagcagaggcagaagaagcagccccagccagagagccggcagccccctgcccagaaacagaggcaaccagaccagaagccccagccccagagagtga

SEQ ID NO: 49 - protein, 474 aa, Artificial Sequence
  Codon optimized HBV HBeAg-Heron HBcAg
MQLFHLCLIISCSCPTVQASKLCLGWLWGLDIDPYKEFGATAELLSFLPSDFFPSVRDLLDTASALYREALESPEHCSPHHTALRQAILCWGELMTLATWVGVNLEDPASRDLVVSYVNTNMGLKFRQLLWFHISCLTFGRETVIEYLVSFGVWIRTPPAYRPPNAPILSTLPETTVVRRRGRSPRRRTPSPRRRRSQSPRRRRSQSRESQCMDVNASRALANVYDLPDDFFPQIDDLVRDAKDALEPYWKAETIKKHVLIATHFVDLIEDFWQTTQGMSQIADALRAVIPPTTVPVPEGFLITHSEAEELPLNDLFSLQEERIVNFQPDYPITARIHTHLRVYTKLNEQALDKARRLLWWHYNCLLWGEATVTNYISRLRTWLSTPEKYRGKDAPTIEAITRPIQVAQGGRNQTKGTRKPRGLEPRRRKVKTTVVYGRRRSKSRGRRSSPSQRAGSPLPRNRGNQTRSPSPRE

SEQ ID NO: 50 - DNA, 1425 bp, Artificial Sequence
  Codon optimized HBV HBeAg-Heron HBcAg
atgcagctgttccacctgtgcctgatcatcagctgcagctgccccaccgtgcaggccagcaagctgtgcctgggctggctgtggggcctggacatcgacccctacaaggagttcggcgccaccgccgagctgctgagcttcctgcccagcgacttcttccccagcgtgagagacctgctggacaccgccagcgccctgtacagagaggccctggagagccccgagcactgcagcccccaccacaccgccctgagacaggccatcctgtgctggggcgagctgatgaccctggccacctgggtgggcgtgaacctggaggaccccgccagcagagacctggtggtgagctacgtgaacaccaacatgggcctgaagttcagacagctgctgtggttccacatcagctgcctgaccttcggcagagagaccgtgatcgagtacctggtgagcttcggcgtgtggatcagaaccccccccgcctacagaccccccaacgcccccatcctgagcaccctgcccgagaccaccgtggtgagaagaagaggcagaagccccagaagaagaacccccagccccagaagaagaagaagccagagccccagaagaagaagaagccagagcagagagagccagtgcatggacgtgaacgccagcagagccctggccaacgtgtacgacctgcccgacgacttcttcccccagatcgacgacctggtgagagacgccaaggacgccctggagccctactggaaggccgagaccatcaagaagcacgtgctgatcgccacccacttcgtggacttgatcgaggacttctggcagaccacccagggcatgagccagatcgccgacgccctgagagccgtgatcccccccaccaccgtgcccgtgcccgagggcttcctgatcacccacagcgaggccgaggagctccccctgaacgacctgttcagtctgcaggaggagagaatcgtgaacttccagcccgactaccccatcaccgccagaattcacacccacctgagagtgtacaccaagctgaacgagcaggccctggacaaggccagaagactgctgtggtggcactacaactgcctgctgtggggcgaggccaccgtgaccaactacatcagcagactgagaacctggctgagcacccccgagaagtacagaggcaaggacgcccccaccatcgaggccatcaccagacccatccaggtggcccagggcggcagaaaccagaccaagggcaccagaaagcccagaggcctggagcccagaagaagaaaggtgaagaccaccgtggtgtacggcagaagaagaagcaagagcagaggcagaagaagcagccccagccagagagccggcagccccctgcccagaaacagaggcaaccagaccagaagccccagccccagagagtga

SEQ ID NO: 51 - protein, 445 aa, Artificial Sequence
  Codon optimized HBV HBcAg-Stork HBcAg
MDIDPYKEFGATVELLSFLPSDFFPSVRDLLDTASALYREALESPEHCSPHHTALRQAILCWGELMTLATWVGVNLEDPASRDLVVSYVNTNMGLKFRQLLWFHISCLTFGRETVIEYLVSFGVWIRTPPAYRPPNAPILSTLPETTVVRRRGRSPRRRTPSPRRRRSQSPRRRRSQSRESQCMDVNASRALANVYDLPDDFFPQIDDLVRDAKDALEPYWKAETIKKHVLIATHFVDLIEDFWQTTQGMSQIADALRAVIPPTTTPVPDGYLISHNEAQELPLNDLFVLQEERIVNFQPDYPITARIHTHLRVYTKLNEQALDKARRLLWWHYNCLLWGESNVTNYISRLRTWLSTPEKYRGKDAPTIEAITRPIQVAQGSRNQTKGVRKPRGLEPRRRKVKTTVVYGRRRSKSRGRRSSPSQRAGSPIPRNRENQSRSSSPRE

SEQ ID NO: 52 - DNA, 1338 bp, Artificial Sequence
  Codon optimized HBV HBcAg-Stork HBcAg
atggacatcgacccctacaaggagttcggcgccaccgtggagctgctgagcttcctgcccagcgacttcttccccagcgtgagagacctgctggacaccgccagcgccctgtacagagaggccctggagagccccgagcactgcagcccccaccacaccgccctgagacaggccatcctgtgctggggcgagctgatgaccctggccacctgggtgggcgtgaacctggaggaccccgccagcagagacctggtggtgagctacgtgaacaccaacatgggcctgaagttcagacagctgctgtggttccacatcagctgcctgaccttcggcagagagaccgtgatcgagtacctggtgagcttcggcgtgtggatcagaaccccccccgcctacagaccccccaacgcccccatcctgagcaccctgcccgagaccaccgtggtgagaagaagaggcagaagccccagaagaagaacccccagccccagaagaagaagaagccagagccccagaagaagaagaagccagagcagagagagccagtgcatggacgtgaacgccagcagagccctggccaacgtgtacgacctgcccgacgacttcttcccccagatcgacgacctggtgagagacgccaaggacgccctggagccctactggaaggccgagaccatcaagaagcacgtgctgatcgccacccacttcgtggacttgatcgaggacttctggcagaccacccagggcatgagccagatcgccgacgccctgagagccgtgatcccccccaccaccacccccgtgcccgacggctacctgatcagccacaacgaggcccaggagctccccctgaacgacctgttcgtgctgcaggaggagagaatcgtgaacttccagcccgactaccccatcaccgccagaattcacacccacctgagagtgtacaccaagctgaacgagcaggccctggacaaggccagaagactgctgtggtggcactacaactgcctgctgtggggcgagagcaacgtgaccaactacatcagcagactgagaacctggctgagcacccccgagaagtacagaggcaaggacgcccccaccatcgaggccatcaccagacccatccaggtggcccagggcagcagaaaccagaccaagggcgtgagaaagcccagaggcctggagcccagaagaagaaaggtgaagaccaccgtggtgtacggcagaagaagaagcaagagcagaggcagaagaagcagccccagccagagagccggcagccccatccccagaaacagagagaaccagagcagaagcagcagccccagagagtga

SEQ ID NO: 53 - protein, 445 aa, Artificial Sequence
  Codon optimized HBV HBcAg-Heron core
MDIDPYKEFGATVELLSFLPSDFFPSVRDLLDTASALYREALESPEHCSPHHTALRQAILCWGELMTLATWVGVNLEDPASRDLVVSYVNTNMGLKFRQLLWFHISCLTFGRETVIEYLVSFGVWIRTPPAYRPPNAPILSTLPETTVVRRRGRSPRRRTPSPRRRRSQSPRRRRSQSRESQCMDVNASRALANVYDLPDDFFPQIDDLVRDAKDALEPYWKAETIKKHVLIATHFVDLIEDFWQTTQGMSQIADALRAVIPPTTVPVPEGFLITHSEAEELPLNDLFSLQEERIVNFQPDYPITARIHTHLRVYTKLNEQALDKARRLLWWHYNCLLWGEATVTNYISRLRTWLSTPEKYRGKDAPTIEAITRPIQVAQGGRNQTKGTRKPRGLEPRRRKVKTTVVYGRRRSKSRGRRSSPSQRAGSPLPRNRGNQTRSPSPRE

SEQ ID NO: 54 - DNA, 1338 bp, Artificial Sequence
  Codon optimized HBV HBcAg-Heron core
atggacatcgacccctacaaggagttcggcgccaccgtggagctgctgagcttcctgcccagcgacttcttccccagcgtgagagacctgctggacaccgccagcgccctgtacagagaggccctggagagccccgagcactgcagcccccaccacaccgccctgagacaggccatcctgtgctggggcgagctgatgaccctggccacctgggtgggcgtgaacctggaggaccccgccagcagagacctggtggtgagctacgtgaacaccaacatgggcctgaagttcagacagctgctgtggttccacatcagctgcctgaccttcggcagagagaccgtgatcgagtacctggtgagcttcggcgtgtggatcagaaccccccccgcctacagaccccccaacgcccccatcctgagcaccctgcccgagaccaccgtggtgagaagaagaggcagaagccccagaagaagaacccccagccccagaagaagaagaagccagagccccagaagaagaagaagccagagcagagagagccagtgcatggacgtgaacgccagcagagccctggccaacgtgtacgacctgcccgacgacttcttcccccagatcgacgacctggtgagagacgccaaggacgccctggagccctactggaaggccgagaccatcaagaagcacgtgctgatcgccacccacttcgtggacttgatcgaggacttctggcagaccacccagggcatgagccagatcgccgacgccctgagagccgtgatcccccccaccaccgtgcccgtgcccgagggcttcctgatcacccacagcgaggccgaggagctccccctgaacgacctgttcagtctgcaggaggagagaatcgtgaacttccagcccgactaccccatcaccgccagaattcacacccacctgagagtgtacaccaagctgaacgagcaggccctggacaaggccagaagactgctgtggtggcactacaactgcctgctgtggggcgaggccaccgtgaccaactacatcagcagactgagaacctggctgagcacccccgagaagtacagaggcaaggacgcccccaccatcgaggccatcaccagacccatccaggtggcccagggcggcagaaaccagaccaagggcaccagaaagcccagaggcctggagcccagaagaagaaaggtgaagaccaccgtggtgtacggcagaagaagaagcaagagcagaggcagaagaagcagccccagccagagagccggcagccccctgcccagaaacagaggcaaccagaccagaagccccagccccagagagtga

SEQ ID NO: 55 - protein, 422 aa, Artificial Sequence
  Codon optimized BetvI (Birch)-Stork HBcAg
MGVFNYETETTSVIPAARLFKAFILDGDNLVPKVAPQAISSVENIEGNGGPGTIKKINFPEGFPFKYVKDRVDEVDHTNFKYNYSVIEGGPVGDTLEKISNEIKIVATPDGGCVLKISNKYHTKGNHEVKAEQVKASKEMGETLLRAVESYLLAHSDAYNMDVNASRALANVYDLPDDFFPQIDDLVRDAKDALEPYWKAETIKKHVLIATHFVDLIEDFWQTTQGMSQIADALRAVIPPTTTPVPDGYLISHNEAQELPLNDLFVLQEERIVNFQPDYPITARIHTHLRVYTKLNEQALDKARRLLWWHYNCLLWGESNVTNYISRLRTWLSTPEKYRGKDAPTIEAITRPIQVAQGSRNQTKGVRKPRGLEPRRRKVKTTVVYGRRRSKSRGRRSSPSQRAGSPIPRNRENQSRSSSPRE

SEQ ID NO: 56 - DNA, 1269 bp, Artificial Sequence
  Codon optimized BetvI (Birch)-Stork HBcAg
atgggcgtcttcaattacgaaaccgagacaactagtgtgatcccagctgcacgcctttttaaggccttcattcttgatggagacaaccttgtgccaaaggtggctccccaagctattagtagcgtcgaaaacatcgagggaaatggcggaccgggaaccatcaaaaagatcaactttccggagggcttccctttcaagtacgttaaggacagggttgatgaagttgatcatacaaattttaagtacaactactccgtgatcgagggcggccccgttggagatactctggaaaagatcagcaacgagattaagatcgtggctacacccgatggcgggtgtgtgcttaagatctccaacaaatatcataccaaagggaaccacgaggttaaggctgaacaggtgaaggcatcaaaggagatgggagagaccctcctccgagcggtggagtcttacttgctcgcacacagcgatgcttacaatatggacgtgaacgccagcagagccctggccaacgtgtacgacctgcccgacgacttcttcccccagatcgacgacctggtgagagacgccaaggacgccctggagccctactggaaggccgagaccatcaagaagcacgtgctgatcgccacccacttcgtggacttgatcgaggacttctggcagaccacccagggcatgagccagatcgccgacgccctgagagccgtgatcccccccaccaccacccccgtgcccgacggctacctgatcagccacaacgaggcccaggagctccccctgaacgacctgttcgtgctgcaggaggagagaatcgtgaacttccagcccgactaccccatcaccgccagaattcacacccacctgagagtgtacaccaagctgaacgagcaggccctggacaaggccagaagactgctgtggtggcactacaactgcctgctgtggggcgagagcaacgtgaccaactacatcagcagactgagaacctggctgagcacccccgagaagtacagaggcaaggacgcccccaccatcgaggccatcaccagacccatccaggtggcccagggcagcagaaaccagaccaagggcgtgagaaagcccagaggcctggagcccagaagaagaaaggtgaagaccaccgtggtgtacggcagaagaagaagcaagagcagaggcagaagaagcagccccagccagagagccggcagccccatccccagaaacagagagaaccagagcagaagcagcagccccagagagtga

SEQ ID NO: 57 - protein, 422 aa, Artificial Sequence
  Codon optimized BetvI (Birch)-Heron HBcAg
MGVFNYETETTSVIPAARLFKAFILDGDNLVPKVAPQAISSVENIEGNGGPGTIKKINFPEGFPFKYVKDRVDEVDHTNFKYNYSVIEGGPVGDTLEKISNEIKIVATPDGGCVLKISNKYHTKGNHEVKAEQVKASKEMGETLLRAVESYLLAHSDAYNMDVNASRALANVYDLPDDFFPQIDDLVRDAKDALEPYWKAETIKKHVLIATHFVDLIEDFWQTTQGMSQIADALRAVIPPTTVPVPEGFLITHSEAEELPLNDLFSLQEERIVNFQPDYPITARIHTHLRVYTKLNEQALDKARRLLWWHYNCLLWGEATVTNYISRLRTWLSTPEKYRGKDAPTIEAITRPIQVAQGGRNQTKGTRKPRGLEPRRRKVKTTVVYGRRRSKSRGRRSSPSQRAGSPLPRNRGNQTRSPSPRE

SEQ ID NO: 58 - DNA, 1269 bp, Artificial Sequence
  Codon optimized BetvI (Birch)-Heron HBcAg
atgggcgtcttcaattacgaaaccgagacaactagtgtgatcccagctgcacgcctttttaaggccttcattcttgatggagacaaccttgtgccaaaggtggctccccaagctattagtagcgtcgaaaacatcgagggaaatggcggaccgggaaccatcaaaaagatcaactttccggagggcttccctttcaagtacgttaaggacagggttgatgaagttgatcatacaaattttaagtacaactactccgtgatcgagggcggccccgttggagatactctggaaaagatcagcaacgagattaagatcgtggctacacccgatggcgggtgtgtgcttaagatctccaacaaatatcataccaaagggaaccacgaggttaaggctgaacaggtgaaggcatcaaaggagatgggagagaccctcctccgagcggtggagtcttacttgctcgcacacagcgatgcttacaatatggacgtgaacgccagcagagccctggccaacgtgtacgacctgcccgacgacttcttcccccagatcgacgacctggtgagagacgccaaggacgccctggagccctactggaaggccgagaccatcaagaagcacgtgctgatcgccacccacttcgtggacttgatcgaggacttctggcagaccacccagggcatgagccagatcgccgacgccctgagagccgtgatcccccccaccaccgtgcccgtgcccgagggcttcctgatcacccacagcgaggccgaggagctccccctgaacgacctgttcagtctgcaggaggagagaatcgtgaacttccagcccgactaccccatcaccgccagaattcacacccacctgagagtgtacaccaagctgaacgagcaggccctggacaaggccagaagactgctgtggtggcactacaactgcctgctgtggggcgaggccaccgtgaccaactacatcagcagactgagaacctggctgagcacccccgagaagtacagaggcaaggacgcccccaccatcgaggccatcaccagacccatccaggtggcccagggcggcagaaaccagaccaagggcaccagaaagcccagaggcctggagcccagaagaagaaaggtgaagaccaccgtggtgtacggcagaagaagaagcaagagcagaggcagaagaagcagccccagccagagagccggcagccccctgcccagaaacagaggcaaccagaccagaagccccagccccagagagtga

SEQ ID NO: 59 - protein, 648 aa, Artificial Sequence
  Codon optimized Ovalbumin-Stork HBcAg
MGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSPMDVNASRALANVYDLPDDFFPQIDDLVRDAKDALEPYWKAETIKKHVLIATHFVDLIEDFWQTTQGMSQIADALRAVIPPTTTPVPDGYLISHNEAQELPLNDLFVLQEERIVNFQPDYPITARIHTHLRVYTKLNEQALDKARRLLWWHYNCLLWGESNVTNYISRLRTWLSTPEKYRGKDAPTIEAITRPIQVAQGSRNQTKGVRKPRGLEPRRRKVKTTVVYGRRRSKSRGRRSSPSQRAGSPIPRNRENQSRSSSPRE

SEQ ID NO: 60 - DNA, 1947 bp, Artificial Sequence
  Codon optimized Ovalbumin-Stork HBcAg
atgggcagcatcggcgccgccagcatggagttctgcttcgacgtgttcaaggagctgaaggtgcaccacgccaacgagaacatcttctactgccccatcgccatcatgagcgccctggccatggtgtacctgggcgccaaggacagcacccgcacccagatcaacaaggtggtgcgcttcgacaagctgcccggcttcggcgacagcatcgaggcccagtgcggcaccagcgtgaacgtgcacagcagcctgcgcgacatcctgaaccagatcaccaagcccaacgacgtgtacagcttcagcctggccagccgcctgtacgccgaggagcgctaccccatcctgcccgagtacctgcagtgcgtgaaggagctgtaccgcggcggcctggagcccatcaacttccagaccgccgccgaccaggcccgcgagctgatcaacagctgggtggagagccagaccaacggcatcatccgcaacgtgctgcagcccagcagcgtggacagccagaccgccatggtgctggtgaacgccatcgtgttcaagggcctgtgggagaaggccttcaaggacgaggacacccaggccatgcccttccgcgtgaccgagcaggagagcaagcccgtgcagatgatgtaccagatcggcctgttccgcgtggccagcatggccagcgagaagatgaagatcctggagctgcccttcgccagcggcaccatgagcatgctggtgctgctgcccgacgaggtgagcggcctggagcagctggagagcatcatcaacttcgagaagctgaccgagtggaccagcagcaacgtgatggaggagcgcaagatcaaggtgtacctgccccgcatgaagatggaggagaagtacaacctgaccagcgtgctgatggccatgggcatcaccgacgtgttcagcagcagcgccaacctgagcggcatcagcagcgccgagagcctgaagatcagccaggccgtgcacgccgcccacgccgagatcaacgaggccggccgcgaggtggtgggcagcgccgaggccggcgtggacgccgccagcgtgagcgaggagttccgcgccgaccaccccttcctgttctgcatcaagcacatcgccaccaacgccgtgctgttcttcggccgctgcgtgagccccatggacgtgaacgccagcagagccctggccaacgtgtacgacctgcccgacgacttcttcccccagatcgacgacctggtgagagacgccaaggacgccctggagccctactggaaggccgagaccatcaagaagcacgtgctgatcgccacccacttcgtggacttgatcgaggacttctggcagaccacccagggcatgagccagatcgccgacgccctgagagccgtgatcccccccaccaccacccccgtgcccgacggctacctgatcagccacaacgaggcccaggagctccccctgaacgacctgttcgtgctgcaggaggagagaatcgtgaacttccagcccgactaccccatcaccgccagaattcacacccacctgagagtgtacaccaagctgaacgagcaggccctggacaaggccagaagactgctgtggtggcactacaactgcctgctgtggggcgagagcaacgtgaccaactacatcagcagactgagaacctggctgagcacccccgagaagtacagaggcaaggacgcccccaccatcgaggccatcaccagacccatccaggtggcccagggcagcagaaaccagaccaagggcgtgagaaagcccagaggcctggagcccagaagaagaaaggtgaagaccaccgtggtgtacggcagaagaagaagcaagagcagaggcagaagaagcagccccagccagagagccggcagccccatccccagaaacagagagaaccagagcagaagcagcagccccagagagtga

SEQ ID NO: 61 - protein, 648 aa, Artificial Sequence
  Codon optimized Ovalbumin-Heron HBcAg
MGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSPMDVNASRALANVYDLPDDFFPQIDDLVRDAKDALEPYWKAETIKKHVLIATHFVDLIEDFWQTTQGMSQIADALRAVIPPTTVPVPEGFLITHSEAEELPLNDLFSLQEERIVNFQPDYPITARIHTHLRVYTKLNEQALDKARRLLWWHYNCLLWGEATVTNYISRLRTWLSTPEKYRGKDAPTIEAITRPIQVAQGGRNQTKGTRKPRGLEPRRRKVKTTVVYGRRRSKSRGRRSSPSQRAGSPLPRNRGNQTRSPSPRE

SEQ ID NO: 62 - DNA, 1947 bp, Artificial Sequence
  Codon optimized Ovalbumin-Heron HBcAg
atgggcagcatcggcgccgccagcatggagttctgcttcgacgtgttcaaggagctgaaggtgcaccacgccaacgagaacatcttctactgccccatcgccatcatgagcgccctggccatggtgtacctgggcgccaaggacagcacccgcacccagatcaacaaggtggtgcgcttcgacaagctgcccggcttcggcgacagcatcgaggcccagtgcggcaccagcgtgaacgtgcacagcagcctgcgcgacatcctgaaccagatcaccaagcccaacgacgtgtacagcttcagcctggccagccgcctgtacgccgaggagcgctaccccatcctgcccgagtacctgcagtgcgtgaaggagctgtaccgcggcggcctggagcccatcaacttccagaccgccgccgaccaggcccgcgagctgatcaacagctgggtggagagccagaccaacggcatcatccgcaacgtgctgcagcccagcagcgtggacagccagaccgccatggtgctggtgaacgccatcgtgttcaagggcctgtgggagaaggccttcaaggacgaggacacccaggccatgcccttccgcgtgaccgagcaggagagcaagcccgtgcagatgatgtaccagatcggcctgttccgcgtggccagcatggccagcgagaagatgaagatcctggagctgcccttcgccagcggcaccatgagcatgctggtgctgctgcccgacgaggtgagcggcctggagcagctggagagcatcatcaacttcgagaagctgaccgagtggaccagcagcaacgtgatggaggagcgcaagatcaaggtgtacctgccccgcatgaagatggaggagaagtacaacctgaccagcgtgctgatggccatgggcatcaccgacgtgttcagcagcagcgccaacctgagcggcatcagcagcgccgagagcctgaagatcagccaggccgtgcacgccgcccacgccgagatcaacgaggccggccgcgaggtggtgggcagcgccgaggccggcgtggacgccgccagcgtgagcgaggagttccgcgccgaccaccccttcctgttctgcatcaagcacatcgccaccaacgccgtgctgttcttcggccgctgcgtgagccccatggacgtgaacgccagcagagccctggccaacgtgtacgacctgcccgacgacttcttcccccagatcgacgacctggtgagagacgccaaggacgccctggagccctactggaaggccgagaccatcaagaagcacgtgctgatcgccacccacttcgtggacttgatcgaggacttctggcagaccacccagggcatgagccagatcgccgacgccctgagagccgtgatcccccccaccaccgtgcccgtgcccgagggcttcctgatcacccacagcgaggccgaggagctccccctgaacgacctgttcagtctgcaggaggagagaatcgtgaacttccagcccgactaccccatcaccgccagaattcacacccacctgagagtgtacaccaagctgaacgagcaggccctggacaaggccagaagactgctgtggtggcactacaactgcctgctgtggggcgaggccaccgtgaccaactacatcagcagactgagaacctggctgagcacccccgagaagtacagaggcaaggacgcccccaccatcgaggccatcaccagacccatccaggtggcccagggcggcagaaaccagaccaagggcaccagaaagcccagaggcctggagcccagaagaagaaaggtgaagaccaccgtggtgtacggcagaagaagaagcaagagcagaggcagaagaagcagccccagccagagagccggcagccccctgcccagaaacagaggcaaccagaccagaagccccagccccagagagtga

SEQ ID NO: 63 - protein, 1417 aa, Artificial Sequence
  Codon opt. NS3/4A-4Bjunct-NS5A-4Bjunct-Stork HBcAg
MAPITAYAQQTRGLLGCIITSLTGRDKNQVEGEVQIVSTAAQTFLATCINGVCWTVYHGAGTRTIASPKGPVIQMYTNVDQDLVGWPAPQGARSLTPCTCGSSDLYLVTRHADVIPVRRRGDGRGSLLSPRPISYLKGSSGGPLLCPAGHAVGIFRAAVCTRGVAKAVDFIPVESLETTMRSPVFSDNSSPPAVPQSYQVAHLHAPTGSGKSTKVPAAYAAQGYKVLVLNPSVAATMGFGAYMSKAHGIDPNIRTGVRTITTGSPITYSTYGKFLADGGCSGGAYDIIICDECHSTDATSILGIGTVLDQAETAGARLTVLATATPPGSVTVPHPNIEEVALSTTGEIPFYGKAIPLEAIKGGRHLIFCHSKKKCDELAAKLVALGVNAVAYYRGLDVSVIPTSGDVVVVATDALMTGFTGDFDSVIDCNTCVTQTVDFSLDPTFTIETITLPQDAVSRTQRRGRTGRGKPGIYRFVAPGERPSGMFDSSVLCECYDAGCAWYELTPAETTVRLRAYMNTPGLPVCQDHLEFWEGVFTGLTHIDAHFLSQTKQSGENLPYLVAYQATVCARAQAPPPSWDQMWKCLIRLKPTLHGPTPLLYRLGAVQNEVTLTHPVTKYIMTCMSADLEVVTSTWVLVGGVLAALAAYCLSTGCVVIVGRIVLSGKPAIIPDREVLYREFDEMEECSQHLPYIEQGMGSGSWLRDVWDWICTVLTDFKTWLQSKLLPKLPGVPFFSCQRGYKGVWRGDGIMQTTCPCGAQITGHVKNGSMRIVGPKTCSNTWHGTFPINAYTTGPCTPSPAPNYSRALWRVAAEEYVEITRVGDFHYVTGMTTDNVKCPCQVPAPEFFTELDGVRLHRYAPACRPLLREDVTFQVGLNQYLVGSQLPCEPEPDVAVLTSMLTDPSHITAETAKRRLARGSPPSLASSSASQLSAPSLKATCTTHHDSPDADLIEANLLWRQEMGGNITRVESENKVVILDSFDPLRAEEDEREVSVAAEILRKSKKFPPALPIWARPDYNPPLLESWKSPDYVPPAVHGCPLPPTTGPPIPPPRKKRTVVLTESTVSSALAELATKTFGSSGSSAVDSGTATAPPDQTSDDGDKESDIESYSSMPPLEGEPGDPDLSDGSWSTVSGEAGDDIVCCSQHLPYIEQGMDVNASRALANVYDLPDDFFPQIDDLVRDAKDALEPYWKAETIKKHVLIATHFVDLIEDFWQTTQGMSQIADALRAVIPPTTTPVPDGYLISHNEAQELPLNDLFVLQEERIVNFQPDYPITARIHTHLRVYTKLNEQALDKARRLLWWHYNCLLWGESNVTNYISRLRTWLSTPEKYRGKDAPTIEAITRPIQVAQGSRNQTKGVRKPRGLEPRRRKVKTTVVYGRRRSKSRGRRSSPSQRAGSPIPRNRENQSRSSSPRE

SEQ ID NO: 64 - DNA, 4254 bp, Artificial Sequence
  Codon opt. NS3/4A-4Bjunct-NS5A-4Bjunct-Stork HBcAg
atggcccccatcaccgcctacgcccagcagacccgcggcctgctgggctgcatcatcaccagcctgaccggccgcgacaagaaccaggtggagggcgaggtgcagatcgtgagcaccgccgcccagaccttcctggccacctgcatcaacggcgtgtgctggaccgtgtaccacggcgccggcacccgcaccatcgccagccccaagggccccgtgatccagatgtacaccaacgtggaccaggacctggtgggctggcccgccccccagggcgcccgcagcctgaccccctgcacctgcggcagcagcgacctgtacctggtgacccgccacgccgacgtgatccccgtgcgccgccgcggcgacggccgcggcagcctgctgagcccccgccccatcagctacctgaagggcagcagcggcggccccctgctgtgccccgccggccacgccgtgggcatcttccgcgccgccgtgtgcacccgcggcgtggccaaggccgtggacttcatccccgtggagagcctggagaccaccatgcgcagccccgtgttcagcgacaacagcagcccccccgccgtgccccagagctaccaggtggcccacctgcacgcccccaccggcagcggcaagagcaccaaggtgcccgccgcctacgccgcccagggctacaaggtgctggtgctgaaccccagcgtggccgccaccatgggcttcggcgcctacatgagcaaggcccacggcatcgaccccaacatccgcaccggcgtgcgcaccatcaccaccggcagccccatcacctacagcacctacggcaagttcctggccgacggcggctgcagcggcggcgcctacgacatcatcatctgcgacgagtgccacagcaccgacgccaccagcatcctgggcatcggcaccgtgctggaccaggccgagaccgccggcgcccgcctgaccgtgctggccaccgccaccccccccggcagcgtgaccgtgccccaccccaacatcgaggaggtggccctgagcaccaccggcgagatccccttctacggcaaggccatccccctggaggccatcaagggcggccgccacctgatcttctgccacagcaagaagaagtgcgacgagctggccgccaagctggtggccctgggcgtgaacgccgtggcctactaccgcggcctggacgtgagcgtgatccccaccagcggcgacgtggtggtggtggccaccgacgccctgatgaccggcttcaccggcgacttcgacagcgtgatcgactgcaacacctgcgtgacccagaccgtggacttcagcctggaccccaccttcaccatcgagaccatcaccctgccccaggacgccgtgagccgcacccagcgccgcggccgcaccggccgcggcaagcccggcatctaccgcttcgtggcccccggcgagcgccccagcggcatgttcgacagcagcgtgctgtgcgagtgctacgacgccggctgcgcctggtacgagctgacccccgccgagaccaccgtgcgcctgcgcgcctacatgaacacccccggcctgcccgtgtgccaggaccacctggagttctgggagggcgtgttcaccggcctgacccacatcgacgcccacttcctgagccagaccaagcagagcggcgagaacctgccctacctggtggcctaccaggccaccgtgtgcgcccgcgcccaggcccccccccccagctgggaccagatgtggaagtgcctgatccgcctgaagcccaccctgcacggccccacccccctgctgtaccgcctgggcgccgtgcagaacgaggtgaccctgacccaccccgtgaccaagtacatcatgacctgcatgagcgccgacctggaggtggtgaccagcacctgggtgctggtgggcggcgtgctggccgccctggccgcctactgcctgagcaccggctgcgtggtgatcgtgggccgcatcgtgctgagcggcaagcccgccatcatccccgaccgcgaggtgctgtaccgcgagttcgacgagatggaggagtgcagccagcacctgccctacatcgagcagggcatgggatctggaagctggctgagggatgtttgggattggatttgtaccgtcctcaccgacttcaaaacctggctccagtccaagctgctgccaaagctgcccggagtgccattcttctcctgtcagaggggctataaaggcgtgtggaggggcgatggaattatgcagactacttgcccctgtggagctcaaattactgggcacgttaagaatggctccatgcggattgttggcccaaaaacctgttccaacacctggcacggaaccttccctattaacgcttacaccaccggaccttgcactccttcccccgcacctaattattcccgggctctctggcgggtggcagcagaggaatatgtcgaaattaccagagtcggcgacttccactacgtcacaggaatgactacagacaacgttaaatgtccctgccaagtgcccgctccagagttctttaccgaactcgacggggttaggctccacagatacgcacccgcctgccggccactgctgcgggaagacgtcacattccaggtcgggctgaaccagtacctggtgggctctcagctgccttgtgagcctgagcccgacgtggcagttctcaccagcatgctcaccgatcctagccacatcaccgctgagacagccaaacgccgcctggctagagggtcccctccctctctggccagctccagcgctagccagctctccgcaccaagcctgaaagccacatgcactacacaccacgatagccccgacgcagacctgattgaagccaacctcctctggagacaggaaatgggcggaaacatcactagggtcgaatccgagaataaagtggttattctggatagcttcgacccactcagggcagaggaagatgagagagaggttagcgtggccgctgagattctccgcaagtccaaaaagttccctcccgcactgcccatttgggcaaggcccgattacaatcctccactgctcgagagctggaagtcccctgactacgtgccaccagccgtccacggatgccctctgccccctaccacaggaccaccaattccaccccctagaaagaaacggaccgtggttctgactgagtccaccgtgtcctctgcactcgctgagctggcaaccaagacctttggatccagcggatcctccgcagtcgactccggcaccgctaccgccccacccgatcaaacctctgacgatggagacaaggagagcgatattgagtcctattccagcatgcccccactcgagggagaacccggcgaccccgacctgagcgatgggtcctggagcactgtgagcggggaagcaggggacgacattgtctgttgcagccagcacctgccctacatcgagcagggcatggacgtgaacgccagcagagccctggccaacgtgtacgacctgcccgacgacttcttcccccagatcgacgacctggtgagagacgccaaggacgccctggagccctactggaaggccgagaccatcaagaagcacgtgctgatcgccacccacttcgtggacttgatcgaggacttctggcagaccacccagggcatgagccagatcgccgacgccctgagagccgtgatcccccccaccaccacccccgtgcccgacggctacctgatcagccacaacgaggcccaggagctccccctgaacgacctgttcgtgctgcaggaggagagaatcgtgaacttccagcccgactaccccatcaccgccagaattcacacccacctgagagtgtacaccaagctgaacgagcaggccctggacaaggccagaagactgctgtggtggcactacaactgcctgctgtggggcgagagcaacgtgaccaactacatcagcagactgagaacctggctgagcacccccgagaagtacagaggcaaggacgcccccaccatcgaggccatcaccagacccatccaggtggcccagggcagcagaaaccagaccaagggcgtgagaaagcccagaggcctggagcccagaagaagaaaggtgaagaccaccgtggtgtacggcagaagaagaagcaagagcagaggcagaagaagcagccccagccagagagccggcagccccatccccagaaacagagagaaccagagcagaagcagcagccccagagagtga

SEQ ID NO: 65 - protein, 1417 aa, Artificial Sequence
  Codon opt. NS3/4A-4Bjunct-NS5A-4Bjunct-Heron HBcAg
MAPITAYAQQTRGLLGCIITSLTGRDKNQVEGEVQIVSTAAQTFLATCINGVCWTVYHGAGTRTIASPKGPVIQMYTNVDQDLVGWPAPQGARSLTPCTCGSSDLYLVTRHADVIPVRRRGDGRGSLLSPRPISYLKGSSGGPLLCPAGHAVGIFRAAVCTRGVAKAVDFIPVESLETTMRSPVFSDNSSPPAVPQSYQVAHLHAPTGSGKSTKVPAAYAAQGYKVLVLNPSVAATMGFGAYMSKAHGIDPNIRTGVRTITTGSPITYSTYGKFLADGGCSGGAYDIIICDECHSTDATSILGIGTVLDQAETAGARLTVLATATPPGSVTVPHPNIEEVALSTTGEIPFYGKAIPLEAIKGGRHLIFCHSKKKCDELAAKLVALGVNAVAYYRGLDVSVIPTSGDVVVVATDALMTGFTGDFDSVIDCNTCVTQTVDFSLDPTFTIETITLPQDAVSRTQRRGRTGRGKPGIYRFVAPGERPSGMFDSSVLCECYDAGCAWYELTPAETTVRLRAYMNTPGLPVCQDHLEFWEGVFTGLTHIDAHFLSQTKQSGENLPYLVAYQATVCARAQAPPPSWDQMWKCLIRLKPTLHGPTPLLYRLGAVQNEVTLTHPVTKYIMTCMSADLEVVTSTWVLVGGVLAALAAYCLSTGCVVIVGRIVLSGKPAIIPDREVLYREFDEMEECSQHLPYIEQGMGSGSWLRDVWDWICTVLTDFKTWLQSKLLPKLPGVPFFSCQRGYKGVWRGDGIMQTTCPCGAQITGHVKNGSMRIVGPKTCSNTWHGTFPINAYTTGPCTPSPAPNYSRALWRVAAEEYVEITRVGDFHYVTGMTTDNVKCPCQVPAPEFFTELDGVRLHRYAPACRPLLREDVTFQVGLNQYLVGSQLPCEPEPDVAVLTSMLTDPSHITAETAKRRLARGSPPSLASSSASQLSAPSLKATCTTHHDSPDADLIEANLLWRQEMGGNITRVESENKVVILDSFDPLRAEEDEREVSVAAEILRKSKKFPPALPIWARPDYNPPLLESWKSPDYVPPAVHGCPLPPTTGPPIPPPRKKRTVVLTESTVSSALAELATKTFGSSGSSAVDSGTATAPPDQTSDDGDKESDIESYSSMPPLEGEPGDPDLSDGSWSTVSGEAGDDIVCCSQHLPYIEQGMDVNASRALANVYDLPDDFFPQIDDLVRDAKDALEPYWKAETIKKHVLIATHFVDLIEDFWQTTQGMSQIADALRAVIPPTTVPVPEGFLITHSEAEELPLNDLFSLQEERIVNFQPDYPITARIHTHLRVYTKLNEQALDKARRLLWWHYNCLLWGEATVTNYISRLRTWLSTPEKYRGKDAPTIEAITRPIQVAQGGRNQTKGTRKPRGLEPRRRKVKTTVVYGRRRSKSRGRRSSPSQRAGSPLPRNRGNQTRSPSPRE

SEQ ID NO: 66 - DNA, 4254 bp, Artificial Sequence
  Codon opt. NS3/4A-4Bjunct-NS5A-4Bjunct-Heron HBcAg
atggcccccatcaccgcctacgcccagcagacccgcggcctgctgggctgcatcatcaccagcctgaccggccgcgacaagaaccaggtggagggcgaggtgcagatcgtgagcaccgccgcccagaccttcctggccacctgcatcaacggcgtgtgctggaccgtgtaccacggcgccggcacccgcaccatcgccagccccaagggccccgtgatccagatgtacaccaacgtggaccaggacctggtgggctggcccgccccccagggcgcccgcagcctgaccccctgcacctgcggcagcagcgacctgtacctggtgacccgccacgccgacgtgatccccgtgcgccgccgcggcgacggccgcggcagcctgctgagcccccgccccatcagctacctgaagggcagcagcggcggccccctgctgtgccccgccggccacgccgtgggcatcttccgcgccgccgtgtgcacccgcggcgtggccaaggccgtggacttcatccccgtggagagcctggagaccaccatgcgcagccccgtgttcagcgacaacagcagcccccccgccgtgccccagagctaccaggtggcccacctgcacgcccccaccggcagcggcaagagcaccaaggtgcccgccgcctacgccgcccagggctacaaggtgctggtgctgaaccccagcgtggccgccaccatgggcttcggcgcctacatgagcaaggcccacggcatcgaccccaacatccgcaccggcgtgcgcaccatcaccaccggcagccccatcacctacagcacctacggcaagttcctggccgacggcggctgcagcggcggcgcctacgacatcatcatctgcgacgagtgccacagcaccgacgccaccagcatcctgggcatcggcaccgtgctggaccaggccgagaccgccggcgcccgcctgaccgtgctggccaccgccaccccccccggcagcgtgaccgtgccccaccccaacatcgaggaggtggccctgagcaccaccggcgagatccccttctacggcaaggccatccccctggaggccatcaagggcggccgccacctgatcttctgccacagcaagaagaagtgcgacgagctggccgccaagctggtggccctgggcgtgaacgccgtggcctactaccgcggcctggacgtgagcgtgatccccaccagcggcgacgtggtggtggtggccaccgacgccctgatgaccggcttcaccggcgacttcgacagcgtgatcgactgcaacacctgcgtgacccagaccgtggacttcagcctggaccccaccttcaccatcgagaccatcaccctgccccaggacgccgtgagccgcacccagcgccgcggccgcaccggccgcggcaagcccggcatctaccgcttcgtggcccccggcgagcgccccagcggcatgttcgacagcagcgtgctgtgcgagtgctacgacgccggctgcgcctggtacgagctgacccccgccgagaccaccgtgcgcctgcgcgcctacatgaacacccccggcctgcccgtgtgccaggaccacctggagttctgggagggcgtgttcaccggcctgacccacatcgacgcccacttcctgagccagaccaagcagagcggcgagaacctgccctacctggtggcctaccaggccaccgtgtgcgcccgcgcccaggcccccccccccagctgggaccagatgtggaagtgcctgatccgcctgaagcccaccctgcacggccccacccccctgctgtaccgcctgggcgccgtgcagaacgaggtgaccctgacccaccccgtgaccaagtacatcatgacctgcatgagcgccgacctggaggtggtgaccagcacctgggtgctggtgggcggcgtgctggccgccctggccgcctactgcctgagcaccggctgcgtggtgatcgtgggccgcatcgtgctgagcggcaagcccgccatcatccccgaccgcgaggtgctgtaccgcgagttcgacgagatggaggagtgcagccagcacctgccctacatcgagcagggcatgggatctggaagctggctgagggatgtttgggattggatttgtaccgtcctcaccgacttcaaaacctggctccagtccaagctgctgccaaagctgcccggagtgccattcttctcctgtcagaggggctataaaggcgtgtggaggggcgatggaattatgcagactacttgcccctgtggagctcaaattactgggcacgttaagaatggctccatgcggattgttggcccaaaaacctgttccaacacctggcacggaaccttccctattaacgcttacaccaccggaccttgcactccttcccccgcacctaattattcccgggctctctggcgggtggcagcagaggaatatgtcgaaattaccagagtcggcgacttccactacgtcacaggaatgactacagacaacgttaaatgtccctgccaagtgcccgctccagagttctttaccgaactcgacggggttaggctccacagatacgcacccgcctgccggccactgctgcgggaagacgtcacattccaggtcgggctgaaccagtacctggtgggctctcagctgccttgtgagcctgagcccgacgtggcagttctcaccagcatgctcaccgatcctagccacatcaccgctgagacagccaaacgccgcctggctagagggtcccctccctctctggccagctccagcgctagccagctctccgcaccaagcctgaaagccacatgcactacacaccacgatagccccgacgcagacctgattgaagccaacctcctctggagacaggaaatgggcggaaacatcactagggtcgaatccgagaataaagtggttattctggatagcttcgacccactcagggcagaggaagatgagagagaggttagcgtggccgctgagattctccgcaagtccaaaaagttccctcccgcactgcccatttgggcaaggcccgattacaatcctccactgctcgagagctggaagtcccctgactacgtgccaccagccgtccacggatgccctctgccccctaccacaggaccaccaattccaccccctagaaagaaacggaccgtggttctgactgagtccaccgtgtcctctgcactcgctgagctggcaaccaagacctttggatccagcggatcctccgcagtcgactccggcaccgctaccgccccacccgatcaaacctctgacgatggagacaaggagagcgatattgagtcctattccagcatgcccccactcgagggagaacccggcgaccccgacctgagcgatgggtcctggagcactgtgagcggggaagcaggggacgacattgtctgttgcagccagcacctgccctacatcgagcagggcatggacgtgaacgccagcagagccctggccaacgtgtacgacctgcccgacgacttcttcccccagatcgacgacctggtgagagacgccaaggacgccctggagccctactggaaggccgagaccatcaagaagcacgtgctgatcgccacccacttcgtggacttgatcgaggacttctggcagaccacccagggcatgagccagatcgccgacgccctgagagccgtgatcccccccaccaccgtgcccgtgcccgagggcttcctgatcacccacagcgaggccgaggagctccccctgaacgacctgttcagtctgcaggaggagagaatcgtgaacttccagcccgactaccccatcaccgccagaattcacacccacctgagagtgtacaccaagctgaacgagcaggccctggacaaggccagaagactgctgtggtggcactacaactgcctgctgtggggcgaggccaccgtgaccaactacatcagcagactgagaacctggctgagcacccccgagaagtacagaggcaaggacgcccccaccatcgaggccatcaccagacccatccaggtggcccagggcggcagaaaccagaccaagggcaccagaaagcccagaggcctggagcccagaagaagaaaggtgaagaccaccgtggtgtacggcagaagaagaagcaagagcagaggcagaagaagcagccccagccagagagccggcagccccctgcccagaaacagaggcaaccagaccagaagccccagccccagagagtga

SEQ ID NO: 67 - protein, 1338 aa, Artificial Sequence
  Codon opt. NS3/4A-4Bjunct-NS5A-4Bjunct-HBV HBcAg
MAPITAYAQQTRGLLGCIITSLTGRDKNQVEGEVQIVSTAAQTFLATCINGVCWTVYHGAGTRTIASPKGPVIQMYTNVDQDLVGWPAPQGARSLTPCTCGSSDLYLVTRHADVIPVRRRGDGRGSLLSPRPISYLKGSSGGPLLCPAGHAVGIFRAAVCTRGVAKAVDFIPVESLETTMRSPVFSDNSSPPAVPQSYQVAHLHAPTGSGKSTKVPAAYAAQGYKVLVLNPSVAATMGFGAYMSKAHGIDPNIRTGVRTITTGSPITYSTYGKFLADGGCSGGAYDIIICDECHSTDATSILGIGTVLDQAETAGARLTVLATATPPGSVTVPHPNIEEVALSTTGEIPFYGKAIPLEAIKGGRHLIFCHSKKKCDELAAKLVALGVNAVAYYRGLDVSVIPTSGDVVVVATDALMTGFTGDFDSVIDCNTCVTQTVDFSLDPTFTIETITLPQDAVSRTQRRGRTGRGKPGIYRFVAPGERPSGMFDSSVLCECYDAGCAWYELTPAETTVRLRAYMNTPGLPVCQDHLEFWEGVFTGLTHIDAHFLSQTKQSGENLPYLVAYQATVCARAQAPPPSWDQMWKCLIRLKPTLHGPTPLLYRLGAVQNEVTLTHPVTKYIMTCMSADLEVVTSTWVLVGGVLAALAAYCLSTGCVVIVGRIVLSGKPAIIPDREVLYREFDEMEECSQHLPYIEQGMGSGSWLRDVWDWICTVLTDFKTWLQSKLLPKLPGVPFFSCQRGYKGVWRGDGIMQTTCPCGAQITGHVKNGSMRIVGPKTCSNTWHGTFPINAYTTGPCTPSPAPNYSRALWRVAAEEYVEITRVGDFHYVTGMTTDNVKCPCQVPAPEFFTELDGVRLHRYAPACRPLLREDVTFQVGLNQYLVGSQLPCEPEPDVAVLTSMLTDPSHITAETAKRRLARGSPPSLASSSASQLSAPSLKATCTTHHDSPDADLIEANLLWRQEMGGNITRVESENKVVILDSFDPLRAEEDEREVSVAAEILRKSKKFPPALPIWARPDYNPPLLESWKSPDYVPPAVHGCPLPPTTGPPIPPPRKKRTVVLTESTVSSALAELATKTFGSSGSSAVDSGTATAPPDQTSDDGDKESDIESYSSMPPLEGEPGDPDLSDGSWSTVSGEAGDDIVCCSQHLPYIEQGMDIDPYKEFGATVELLSFLPSDFFPSVRDLLDTASALYREALESPEHCSPHHTALRQAILCWGELMTLATWVGVNLEDPASRDLVVSYVNTNMGLKFRQLLWFHISCLTFGRETVIEYLVSFGVWIRTPPAYRPPNAPILSTLPETTVVRRRGRSPRRRTPSPRRRRSQSPRRRRSQSRESQC

SEQ ID NO: 68 - DNA, 4017 bp, Artificial Sequence
  Codon opt. NS3/4A-4Bjunct-NS5A-4Bjunct-HBV HBcAg
atggcccccatcaccgcctacgcccagcagacccgcggcctgctgggctgcatcatcaccagcctgaccggccgcgacaagaaccaggtggagggcgaggtgcagatcgtgagcaccgccgcccagaccttcctggccacctgcatcaacggcgtgtgctggaccgtgtaccacggcgccggcacccgcaccatcgccagccccaagggccccgtgatccagatgtacaccaacgtggaccaggacctggtgggctggcccgccccccagggcgcccgcagcctgaccccctgcacctgcggcagcagcgacctgtacctggtgacccgccacgccgacgtgatccccgtgcgccgccgcggcgacggccgcggcagcctgctgagcccccgccccatcagctacctgaagggcagcagcggcggccccctgctgtgccccgccggccacgccgtgggcatcttccgcgccgccgtgtgcacccgcggcgtggccaaggccgtggacttcatccccgtggagagcctggagaccaccatgcgcagccccgtgttcagcgacaacagcagcccccccgccgtgccccagagctaccaggtggcccacctgcacgcccccaccggcagcggcaagagcaccaaggtgcccgccgcctacgccgcccagggctacaaggtgctggtgctgaaccccagcgtggccgccaccatgggcttcggcgcctacatgagcaaggcccacggcatcgaccccaacatccgcaccggcgtgcgcaccatcaccaccggcagccccatcacctacagcacctacggcaagttcctggccgacggcggctgcagcggcggcgcctacgacatcatcatctgcgacgagtgccacagcaccgacgccaccagcatcctgggcatcggcaccgtgctggaccaggccgagaccgccggcgcccgcctgaccgtgctggccaccgccaccccccccggcagcgtgaccgtgccccaccccaacatcgaggaggtggccctgagcaccaccggcgagatccccttctacggcaaggccatccccctggaggccatcaagggcggccgccacctgatcttctgccacagcaagaagaagtgcgacgagctggccgccaagctggtggccctgggcgtgaacgccgtggcctactaccgcggcctggacgtgagcgtgatccccaccagcggcgacgtggtggtggtggccaccgacgccctgatgaccggcttcaccggcgacttcgacagcgtgatcgactgcaacacctgcgtgacccagaccgtggacttcagcctggaccccaccttcaccatcgagaccatcaccctgccccaggacgccgtgagccgcacccagcgccgcggccgcaccggccgcggcaagcccggcatctaccgcttcgtggcccccggcgagcgccccagcggcatgttcgacagcagcgtgctgtgcgagtgctacgacgccggctgcgcctggtacgagctgacccccgccgagaccaccgtgcgcctgcgcgcctacatgaacacccccggcctgcccgtgtgccaggaccacctggagttctgggagggcgtgttcaccggcctgacccacatcgacgcccacttcctgagccagaccaagcagagcggcgagaacctgccctacctggtggcctaccaggccaccgtgtgcgcccgcgcccaggcccccccccccagctgggaccagatgtggaagtgcctgatccgcctgaagcccaccctgcacggccccacccccctgctgtaccgcctgggcgccgtgcagaacgaggtgaccctgacccaccccgtgaccaagtacatcatgacctgcatgagcgccgacctggaggtggtgaccagcacctgggtgctggtgggcggcgtgctggccgccctggccgcctactgcctgagcaccggctgcgtggtgatcgtgggccgcatcgtgctgagcggcaagcccgccatcatccccgaccgcgaggtgctgtaccgcgagttcgacgagatggaggagtgcagccagcacctgccctacatcgagcagggcatgggatctggaagctggctgagggatgtttgggattggatttgtaccgtcctcaccgacttcaaaacctggctccagtccaagctgctgccaaagctgcccggagtgccattcttctcctgtcagaggggctataaaggcgtgtggaggggcgatggaattatgcagactacttgcccctgtggagctcaaattactgggcacgttaagaatggctccatgcggattgttggcccaaaaacctgttccaacacctggcacggaaccttccctattaacgcttacaccaccggaccttgcactccttcccccgcacctaattattcccgggctctctggcgggtggcagcagaggaatatgtcgaaattaccagagtcggcgacttccactacgtcacaggaatgactacagacaacgttaaatgtccctgccaagtgcccgctccagagttctttaccgaactcgacggggttaggctccacagatacgcacccgcctgccggccactgctgcgggaagacgtcacattccaggtcgggctgaaccagtacctggtgggctctcagctgccttgtgagcctgagcccgacgtggcagttctcaccagcatgctcaccgatcctagccacatcaccgctgagacagccaaacgccgcctggctagagggtcccctccctctctggccagctccagcgctagccagctctccgcaccaagcctgaaagccacatgcactacacaccacgatagccccgacgcagacctgattgaagccaacctcctctggagacaggaaatgggcggaaacatcactagggtcgaatccgagaataaagtggttattctggatagcttcgacccactcagggcagaggaagatgagagagaggttagcgtggccgctgagattctccgcaagtccaaaaagttccctcccgcactgcccatttgggcaaggcccgattacaatcctccactgctcgagagctggaagtcccctgactacgtgccaccagccgtccacggatgccctctgccccctaccacaggaccaccaattccaccccctagaaagaaacggaccgtggttctgactgagtccaccgtgtcctctgcactcgctgagctggcaaccaagacctttggatccagcggatcctccgcagtcgactccggcaccgctaccgccccacccgatcaaacctctgacgatggagacaaggagagcgatattgagtcctattccagcatgcccccactcgagggagaacccggcgaccccgacctgagcgatgggtcctggagcactgtgagcggggaagcaggggacgacattgtctgttgcagccagcacctgccctacatcgagcagggcatggacatcgacccctacaaggagttcggcgccaccgtggagctgctgagcttcctgcccagcgacttcttccccagcgtgagagacctgctggacaccgccagcgccctgtacagagaggccctggagagccccgagcactgcagcccccaccacaccgccctgagacaggccatcctgtgctggggcgagctgatgaccctggccacctgggtgggcgtgaacctggaggaccccgccagcagagacctggtggtgagctacgtgaacaccaacatgggcctgaagttcagacagctgctgtggttccacatcagctgcctgaccttcggcagagagaccgtgatcgagtacctggtgagcttcggcgtgtggatcagaaccccccccgcctacagaccccccaacgcccccatcctgagcaccctgcccgagaccaccgtggtgagaagaagaggcagaagccccagaagaagaacccccagccccagaagaagaagaagccagagccccagaagaagaagaagccagagcagagagagccagtgctag

SEQ ID NO: 69 - protein, 262 aa, Artificial Sequence; identical to SEQ ID NO: 19
  Codon optimized Stork HBcAg with HBV HBcAg

SEQ ID NO: 70 - protein, 262 aa, Artificial Sequence; identical to SEQ ID NO: 21
  Codon optimized Stork HBcAg with HBV HBcAg

SEQ ID NO: 71 - DNA, 552 bp, HBV
  misc_feature (0)...(0): wild-type HBcAg
atggacatcgacccttataaagaatttggagctactgtggagttactctcgtttttgccctccgacttctttccttcagtacgagatcttctagataccgcctcagctctgtatcgggaagccttagagtctcctgagcattgttcacctcaccatactgcactcaggcaagcaattctttgctggggggaactaatgactctagctacctgggtgggtgttaatttggaagatccagcgtctagagacctagtagtcagttatgtcaacactaatatgggcctaaagttcaggcaactcttgtggtttcacatttcttgtctcacttttggaagagaaacagttatagagtatttggtgtctttcggagtgtggattcgcactcctccagcttatagaccaccaaatgcccctatcctatcaacacttccggagactactgttgttagacgacgaggcaggtcccctagaagaagaactccctcgcctcgcagacgaaggtctcaatcgccgcgtcgcagaagatctcaatctcgggaatctcaatgttag

SEQ ID NO: 72 - protein, 869 aa, Artificial Sequence
  CONSTR-1 NS3/4A-HBcAg
MAPITAYAQQTRGLLGCIITSLTGRDKNQVEGEVQIVSTAAQTFLATCINGVCWTVYHGAGTRTIASPKGPVIQMYTNVDQDLVGWPAPQGARSLTPCTCGSSDLYLVTRHADVIPVRRRGDGRGSLLSPRPISYLKGSSGGPLLCPAGHAVGIFRAAVCTRGVAKAVDFIPVESLETTMRSPVFSDNSSPPAVPQSYQVAHLHAPTGSGKSTKVPAAYAAQGYKVLVLNPSVAATMGFGAYMSKAHGIDPNIRTGVRTITTGSPITYSTYGKFLADGGCSGGAYDIIICDECHSTDATSILGIGTVLDQAETAGARLTVLATATPPGSVTVPHPNIEEVALSTTGEIPFYGKAIPLEAIKGGRHLIFCHSKKKCDELAAKLVALGVNAVAYYRGLDVSVIPTSGDVVVVATDALMTGFTGDFDSVIDCNTCVTQTVDFSLDPTFTIETITLPQDAVSRTQRRGRTGRGKPGIYRFVAPGERPSGMFDSSVLCECYDAGCAWYELTPAETTVRLRAYMNTPGLPVCQDHLEFWEGVFTGLTHIDAHFLSQTKQSGENLPYLVAYQATVCARAQAPPPSWDQMWKCLIRLKPTLHGPTPLLYRLGAVQNEVTLTHPVTKYIMTCMSADLEVVTPTWVLVGGVLAALAAYCLSTGCVVIVGRIVLSGKPAIIPDREVLYREFDEMEECMDIDPYKEFGATVELLSFLPSDFFPSVRDLLDTASALYREALESPEHCSPHHTALRQAILCWGELMTLATWVGVNLEDPASRDLVVSYVNTNMGLKFRQLLWFHISCLTFGRETVIEYLVSFGVWIRTPPAYRPPNAPILSTLPETTVVRRRGRSPRRRTPSPRRRRSQSPRRRRSQSRESQC

SEQ ID NO: 73 - DNA, 2610 bp, Artificial Sequence
  CONSTR-1 NS3/4A-HBcAg
atggcccccatcaccgcctacgcccagcagacccgcggcctgctgggctgcatcatcaccagcctgaccggccgcgacaagaaccaggtggagggcgaggtgcagatcgtgagcaccgccgcccagaccttcctggccacctgcatcaacggcgtgtgctggaccgtgtaccacggcgccggcacccgcaccatcgccagccccaagggccccgtgatccagatgtacaccaacgtggaccaggacctggtgggctggcccgccccccagggcgcccgcagcctgaccccctgcacctgcggcagcagcgacctgtacctggtgacccgccacgccgacgtgatccccgtgcgccgccgcggcgacggccgcggcagcctgctgagcccccgccccatcagctacctgaagggcagcagcggcggccccctgctgtgccccgccggccacgccgtgggcatcttccgcgccgccgtgtgcacccgcggcgtggccaaggccgtggacttcatccccgtggagagcctggagaccaccatgcgcagccccgtgttcagcgacaacagcagcccccccgccgtgccccagagctaccaggtggcccacctgcacgcccccaccggcagcggcaagagcaccaaggtgcccgccgcctacgccgcccagggctacaaggtgctggtgctgaaccccagcgtggccgccaccatgggcttcggcgcctacatgagcaaggcccacggcatcgaccccaacatccgcaccggcgtgcgcaccatcaccaccggcagccccatcacctacagcacctacggcaagttcctggccgacggcggctgcagcggcggcgcctacgacatcatcatctgcgacgagtgccacagcaccgacgccaccagcatcctgggcatcggcaccgtgctggaccaggccgagaccgccggcgcccgcctgaccgtgctggccaccgccaccccccccggcagcgtgaccgtgccccaccccaacatcgaggaggtggccctgagcaccaccggcgagatccccttctacggcaaggccatccccctggaggccatcaagggcggccgccacctgatcttctgccacagcaagaagaagtgcgacgagctggccgccaagctggtggccctgggcgtgaacgccgtggcctactaccgcggcctggacgtgagcgtgatccccaccagcggcgacgtggtggtggtggccaccgacgccctgatgaccggcttcaccggcgacttcgacagcgtgatcgactgcaacacctgcgtgacccagaccgtggacttcagcctggaccccaccttcaccatcgagaccatcaccctgccccaggacgccgtgagccgcacccagcgccgcggccgcaccggccgcggcaagcccggcatctaccgcttcgtggcccccggcgagcgccccagcggcatgttcgacagcagcgtgctgtgcgagtgctacgacgccggctgcgcctggtacgagctgacccccgccgagaccaccgtgcgcctgcgcgcctacatgaacacccccggcctgcccgtgtgccaggaccacctggagttctgggagggcgtgttcaccggcctgacccacatcgacgcccacttcctgagccagaccaagcagagcggcgagaacctgccctacctggtggcctaccaggccaccgtgtgcgcccgcgcccaggcccccccccccagctgggaccagatgtggaagtgcctgatccgcctgaagcccaccctgcacggccccacccccctgctgtaccgcctgggcgccgtgcagaacgaggtgaccctgacccaccccgtgaccaagtacatcatgacctgcatgagcgccgacctggaggtggtgacccccacctgggtgctggtgggcggcgtgctggccgccctggccgcctactgcctgagcaccggctgcgtggtgatcgtgggccgcatcgtgctgagcggcaagcccgccatcatccccgaccgcgaggtgctgtaccgcgagttcgacgagatggaggagtgcatggacatcgacccctacaaggagttcggcgccaccgtggagctgctgagcttcctgcccagcgacttcttccccagcgtgcgcgacctgctggacaccgccagcgccctgtaccgcgaggccctggagagccccgagcactgcagcccccaccacaccgccctgcgccaggccatcctgtgctggggcgagctgatgaccctggccacctgggtgggcgtgaacctggaggaccccgccagccgcgacctggtggtgagctacgtgaacaccaacatgggcctgaagttccgccagctgctgtggttccacatcagctgcctgaccttcggccgcgagaccgtgatcgagtacctggtgagcttcggcgtgtggatccgcaccccccccgcctaccgcccccccaacgcccccatcctgagcaccctgcccgagaccaccgtggtgcgccgccgcggccgcagcccccgccgccgcacccccagcccccgccgccgccgcagccagagcccccgccgccgccgcagccagagccgcgagagccagtgctag

SEQ ID NO: 74 - protein, 869 aa, Artificial Sequence
  CONSTR-2 Mutant(catalytic triade)NS3/4A-HBcAg
MAPITAYAQQTRGLLGCIITSLTGRDKNQVEGEVQIVSTAAQTFLATCINGVCWTVYAGAGTRTIASPKGPVIQMYTNVDQALVGWPAPQGARSLTPCTCGSSDLYLVTRHADVIPVRRRGDGRGSLLSPRPISYLKGSSGGPLLCPAGHAVGIFRAAVCTRGVAKAVDFIPVESLETTMRSPVFSDNSSPPAVPQSYQVAHLHAPTGSGKSTKVPAAYAAQGYKVLVLNPSVAATMGFGAYMSKAHGIDPNIRTGVRTITTGSPITYSTYGKFLADGGCSGGAYDIIICDECHSTDATSILGIGTVLDQAETAGARLTVLATATPPGSVTVPHPNIEEVALSTTGEIPFYGKAIPLEAIKGGRHLIFCHSKKKCDELAAKLVALGVNAVAYYRGLDVSVIPTSGDVVVVATDALMTGFTGDFDSVIDCNTCVTQTVDFSLDPTFTIETITLPQDAVSRTQRRGRTGRGKPGIYRFVAPGERPSGMFDSSVLCECYDAGCAWYELTPAETTVRLRAYMNTPGLPVCQDHLEFWEGVFTGLTHIDAHFLSQTKQSGENLPYLVAYQATVCARAQAPPPSWDQMWKCLIRLKPTLHGPTPLLYRLGAVQNEVTLTHPVTKYIMTCMSADLEVVTSTWVLVGGVLAALAAYCLSTGCVVIVGRIVLSGKPAIIPDREVLYREFDEMEECMDIDPYKEFGATVELLSFLPSDFFPSVRDLLDTASALYREALESPEHCSPHHTALRQAILCWGELMTLATWVGVNLEDPASRDLVVSYVNTNMGLKFRQLLWFHISCLTFGRETVIEYLVSFGVWIRTPPAYRPPNAPILSTLPETTVVRRRGRSPRRRTPSPRRRRSQSPRRRRSQSRESQC

SEQ ID NO: 75 - DNA, 2610 bp, Artificial Sequence
  CONSTR-2 Mutant(catalytic triade)NS3/4A-HBcAg
atggcccccatcaccgcctacgcccagcagacccgcggcctgctgggctgcatcatcaccagcctgaccggccgcgacaagaaccaggtggagggcgaggtgcagatcgtgagcaccgccgcccagaccttcctggccacctgcatcaacggcgtgtgctggaccgtgtacgccggcgccggcacccgcaccatcgccagccccaagggccccgtgatccagatgtacaccaacgtggaccaggccctggtgggctggcccgccccccagggcgcccgcagcctgaccccctgcacctgcggcagcagcgacctgtacctggtgacccgccacgccgacgtgatccccgtgcgccgccgcggcgacggccgcggcagcctgctgagcccccgccccatcagctacctgaagggcagcagcggcggccccctgctgtgccccgccggccacgccgtgggcatcttccgcgccgccgtgtgcacccgcggcgtggccaaggccgtggacttcatccccgtggagagcctggagaccaccatgcgcagccccgtgttcagcgacaacagcagcccccccgccgtgccccagagctaccaggtggcccacctgcacgcccccaccggcagcggcaagagcaccaaggtgcccgccgcctacgccgcccagggctacaaggtgctggtgctgaaccccagcgtggccgccaccatgggcttcggcgcctacatgagcaaggcccacggcatcgaccccaacatccgcaccggcgtgcgcaccatcaccaccggcagccccatcacctacagcacctacggcaagttcctggccgacggcggctgcagcggcggcgcctacgacatcatcatctgcgacgagtgccacagcaccgacgccaccagcatcctgggcatcggcaccgtgctggaccaggccgagaccgccggcgcccgcctgaccgtgctggccaccgccaccccccccggcagcgtgaccgtgccccaccccaacatcgaggaggtggccctgagcaccaccggcgagatccccttctacggcaaggccatccccctggaggccatcaagggcggccgccacctgatcttctgccacagcaagaagaagtgcgacgagctggccgccaagctggtggccctgggcgtgaacgccgtggcctactaccgcggcctggacgtgagcgtgatccccaccagcggcgacgtggtggtggtggccaccgacgccctgatgaccggcttcaccggcgacttcgacagcgtgatcgactgcaacacctgcgtgacccagaccgtggacttcagcctggaccccaccttcaccatcgagaccatcaccctgccccaggacgccgtgagccgcacccagcgccgcggccgcaccggccgcggcaagcccggcatctaccgcttcgtggcccccggcgagcgccccagcggcatgttcgacagcagcgtgctgtgcgagtgctacgacgccggctgcgcctggtacgagctgacccccgccgagaccaccgtgcgcctgcgcgcctacatgaacacccccggcctgcccgtgtgccaggaccacctggagttctgggagggcgtgttcaccggcctgacccacatcgacgcccacttcctgagccagaccaagcagagcggcgagaacctgccctacctggtggcctaccaggccaccgtgtgcgcccgcgcccaggcccccccccccagctgggaccagatgtggaagtgcctgatccgcctgaagcccaccctgcacggccccacccccctgctgtaccgcctgggcgccgtgcagaacgaggtgaccctgacccaccccgtgaccaagtacatcatgacctgcatgagcgccgacctggaggtggtgaccagcacctgggtgctggtgggcggcgtgctggccgccctggccgcctactgcctgagcaccggctgcgtggtgatcgtgggccgcatcgtgctgagcggcaagcccgccatcatccccgaccgcgaggtgctgtaccgcgagttcgacgagatggaggagtgcatggacatcgacccctacaaggagttcggcgccaccgtggagctgctgagcttcctgcccagcgacttcttccccagcgtgcgcgacctgctggacaccgccagcgccctgtaccgcgaggccctggagagccccgagcactgcagcccccaccacaccgccctgcgccaggccatcctgtgctggggcgagctgatgaccctggccacctgggtgggcgtgaacctggaggaccccgccagccgcgacctggtggtgagctacgtgaacaccaacatgggcctgaagttccgccagctgctgtggttccacatcagctgcctgaccttcggccgcgagaccgtgatcgagtacctggtgagcttcggcgtgtggatccgcaccccccccgcctaccgcccccccaacgcccccatcctgagcaccctgcccgagaccaccgtggtgcgccgccgcggccgcagcccccgccgccgcacccccagcccccgccgccgccgcagccagagcccccgccgccgccgcagccagagccgcgagagccagtgctag

SEQ ID NO: 76 - protein, 869 aa, Artificial Sequence
  CONSTR-3 NS3/4A-HBcAg (NS3 and NS4A-HBcAg fusion)
MAPITAYAQQTRGLLGCIITSLTGRDKNQVEGEVQIVSTAAQTFLATCINGVCWTVYHGAGTRTIASPKGPVIQMYTNVDQDLVGWPAPQGARSLTPCTCGSSDLYLVTRHADVIPVRRRGDGRGSLLSPRPISYLKGSSGGPLLCPAGHAVGIFRAAVCTRGVAKAVDFIPVESLETTMRSPVFSDNSSPPAVPQSYQVAHLHAPTGSGKSTKVPAAYAAQGYKVLVLNPSVAATMGFGAYMSKAHGIDPNIRTGVRTITTGSPITYSTYGKFLADGGCSGGAYDIIICDECHSTDATSILGIGTVLDQAETAGARLTVLATATPPGSVTVPHPNIEEVALSTTGEIPFYGKAIPLEAIKGGRHLIFCHSKKKCDELAAKLVALGVNAVAYYRGLDVSVIPTSGDVVVVATDALMTGFTGDFDSVIDCNTCVTQTVDFSLDPTFTIETITLPQDAVSRTQRRGRTGRGKPGIYRFVAPGERPSGMFDSSVLCECYDAGCAWYELTPAETTVRLRAYMNTPGLPVCQDHLEFWEGVFTGLTHIDAHFLSQTKQSGENLPYLVAYQATVCARAQAPPPSWDQMWKCLIRLKPTLHGPTPLLYRLGAVQNEVTLTHPVTKYIMTCMSADLEVVTSTWVLVGGVLAALAAYCLSTGCVVIVGRIVLSGKPAIIPDREVLYREFDEMEECMDIDPYKEFGATVELLSFLPSDFFPSVRDLLDTASALYREALESPEHCSPHHTALRQAILCWGELMTLATWVGVNLEDPASRDLVVSYVNTNMGLKFRQLLWFHISCLTFGRETVIEYLVSFGVWIRTPPAYRPPNAPILSTLPETTVVRRRGRSPRRRTPSPRRRRSQSPRRRRSQSRESQC

SEQ ID NO: 77 - DNA, 2610 bp, Artificial Sequence
  CONSTR-3 NS3/4A-HBcAg (NS3 and NS4A-HBcAg fusion)
atggcccccatcaccgcctacgcccagcagacccgcggcctgctgggctgcatcatcaccagcctgaccggccgcgacaagaaccaggtggagggcgaggtgcagatcgtgagcaccgccgcccagaccttcctggccacctgcatcaacggcgtgtgctggaccgtgtaccacggcgccggcacccgcaccatcgccagccccaagggccccgtgatccagatgtacaccaacgtggaccaggacctggtgggctggcccgccccccagggcgcccgcagcctgaccccctgcacctgcggcagcagcgacctgtacctggtgacccgccacgccgacgtgatccccgtgcgccgccgcggcgacggccgcggcagcctgctgagcccccgccccatcagctacctgaagggcagcagcggcggccccctgctgtgccccgccggccacgccgtgggcatcttccgcgccgccgtgtgcacccgcggcgtggccaaggccgtggacttcatccccgtggagagcctggagaccaccatgcgcagccccgtgttcagcgacaacagcagcccccccgccgtgccccagagctaccaggtggcccacctgcacgcccccaccggcagcggcaagagcaccaaggtgcccgccgcctacgccgcccagggctacaaggtgctggtgctgaaccccagcgtggccgccaccatgggcttcggcgcctacatgagcaaggcccacggcatcgaccccaacatccgcaccggcgtgcgcaccatcaccaccggcagccccatcacctacagcacctacggcaagttcctggccgacggcggctgcagcggcggcgcctacgacatcatcatctgcgacgagtgccacagcaccgacgccaccagcatcctgggcatcggcaccgtgctggaccaggccgagaccgccggcgcccgcctgaccgtgctggccaccgccaccccccccggcagcgtgaccgtgccccaccccaacatcgaggaggtggccctgagcaccaccggcgagatccccttctacggcaaggccatccccctggaggccatcaagggcggccgccacctgatcttctgccacagcaagaagaagtgcgacgagctggccgccaagctggtggccctgggcgtgaacgccgtggcctactaccgcggcctggacgtgagcgtgatccccaccagcggcgacgtggtggtggtggccaccgacgccctgatgaccggcttcaccggcgacttcgacagcgtgatcgactgcaacacctgcgtgacccagaccgtggacttcagcctggaccccaccttcaccatcgagaccatcaccctgccccaggacgccgtgagccgcacccagcgccgcggccgcaccggccgcggcaagcccggcatctaccgcttcgtggcccccggcgagcgccccagcggcatgttcgacagcagcgtgctgtgcgagtgctacgacgccggctgcgcctggtacgagctgacccccgccgagaccaccgtgcgcctgcgcgcctacatgaacacccccggcctgcccgtgtgccaggaccacctggagttctgggagggcgtgttcaccggcctgacccacatcgacgcccacttcctgagccagaccaagcagagcggcgagaacctgccctacctggtggcctaccaggccaccgtgtgcgcccgcgcccaggcccccccccccagctgggaccagatgtggaagtgcctgatccgcctgaagcccaccctgcacggccccacccccctgctgtaccgcctgggcgccgtgcagaacgaggtgaccctgacccaccccgtgaccaagtacatcatgacctgcatgagcgccgacctggaggtggtgaccagcacctgggtgctggtgggcggcgtgctggccgccctggccgcctactgcctgagcaccggctgcgtggtgatcgtgggccgcatcgtgctgagcggcaagcccgccatcatccccgaccgcgaggtgctgtaccgcgagttcgacgagatggaggagtgcatggacatcgacccctacaaggagttcggcgccaccgtggagctgctgagcttcctgcccagcgacttcttccccagcgtgcgcgacctgctggacaccgccagcgccctgtaccgcgaggccctggagagccccgagcactgcagcccccaccacaccgccctgcgccaggccatcctgtgctggggcgagctgatgaccctggccacctgggtgggcgtgaacctggaggaccccgccagccgcgacctggtggtgagctacgtgaacaccaacatgggcctgaagttccgccagctgctgtggttccacatcagctgcctgaccttcggccgcgagaccgtgatcgagtacctggtgagcttcggcgtgtggatccgcaccccccccgcctaccgcccccccaacgcccccatcctgagcaccctgcccgagaccaccgtggtgcgccgccgcggccgcagcccccgccgccgcacccccagcccccgccgccgccgcagccagagcccccgccgccgccgcagccagagccgcgagagccagtgctag

SEQ ID NO: 78 - protein, 879 aa, Artificial Sequence
  CONSTR-4 NS3/4A-4Bjunct-HBcAg
MAPITAYAQQTRGLLGCIITSLTGRDKNQVEGEVQIVSTAAQTFLATCINGVCWTVYHGAGTRTIASPKGPVIQMYTNVDQDLVGWPAPQGARSLTPCTCGSSDLYLVTRHADVIPVRRRGDGRGSLLSPRPISYLKGSSGGPLLCPAGHAVGIFRAAVCTRGVAKAVDFIPVESLETTMRSPVFSDNSSPPAVPQSYQVAHLHAPTGSGKSTKVPAAYAAQGYKVLVLNPSVAATMGFGAYMSKAHGIDPNIRTGVRTITTGSPITYSTYGKFLADGGCSGGAYDIIICDECHSTDATSILGIGTVLDQAETAGARLTVLATATPPGSVTVPHPNIEEVALSTTGEIPFYGKAIPLEAIKGGRHLIFCHSKKKCDELAAKLVALGVNAVAYYRGLDVSVIPTSGDVVVVATDALMTGFTGDFDSVIDCNTCVTQTVDFSLDPTFTIETITLPQDAVSRTQRRGRTGRGKPGIYRFVAPGERPSGMFDSSVLCECYDAGCAWYELTPAETTVRLRAYMNTPGLPVCQDHLEFWEGVFTGLTHIDAHFLSQTKQSGENLPYLVAYQATVCARAQAPPPSWDQMWKCLIRLKPTLHGPTPLLYRLGAVQNEVTLTHPVTKYIMTCMSADLEVVTSTWVLVGGVLAALAAYCLSTGCVVIVGRIVLSGKPAIIPDREVLYREFDEMEECSQHLPYIEQGMDIDPYKEFGATVELLSFLPSDFFPSVRDLLDTASALYREALESPEHCSPHHTALRQAILCWGELMTLATWVGVNLEDPASRDLVVSYVNTNMGLKFRQLLWFHISCLTFGRETVIEYLVSFGVWIRTPPAYRPPNAPILSTLPETTVVRRRGRSPRRRTPSPRRRRSQSPRRRRSQSRESQC

SEQ ID NO: 79 - DNA, 2640 bp, Artificial Sequence
  CONSTR-4 NS3/4A-4Bjunct-HBcAg
atggcccccatcaccgcctacgcccagcagacccgcggcctgctgggctgcatcatcaccagcctgaccggccgcgacaagaaccaggtggagggcgaggtgcagatcgtgagcaccgccgcccagaccttcctggccacctgcatcaacggcgtgtgctggaccgtgtaccacggcgccggcacccgcaccatcgccagccccaagggccccgtgatccagatgtacaccaacgtggaccaggacctggtgggctggcccgccccccagggcgcccgcagcctgaccccctgcacctgcggcagcagcgacctgtacctggtgacccgccacgccgacgtgatccccgtgcgccgccgcggcgacggccgcggcagcctgctgagcccccgccccatcagctacctgaagggcagcagcggcggccccctgctgtgccccgccggccacgccgtgggcatcttccgcgccgccgtgtgcacccgcggcgtggccaaggccgtggacttcatccccgtggagagcctggagaccaccatgcgcagccccgtgttcagcgacaacagcagcccccccgccgtgccccagagctaccaggtggcccacctgcacgcccccaccggcagcggcaagagcaccaaggtgcccgccgcctacgccgcccagggctacaaggtgctggtgctgaaccccagcgtggccgccaccatgggcttcggcgcctacatgagcaaggcccacggcatcgaccccaacatccgcaccggcgtgcgcaccatcaccaccggcagccccatcacctacagcacctacggcaagttcctggccgacggcggctgcagcggcggcgcctacgacatcatcatctgcgacgagtgccacagcaccgacgccaccagcatcctgggcatcggcaccgtgctggaccaggccgagaccgccggcgcccgcctgaccgtgctggccaccgccaccccccccggcagcgtgaccgtgccccaccccaacatcgaggaggtggccctgagcaccaccggcgagatccccttctacggcaaggccatccccctggaggccatcaagggcggccgccacctgatcttctgccacagcaagaagaagtgcgacgagctggccgccaagctggtggccctgggcgtgaacgccgtggcctactaccgcggcctggacgtgagcgtgatccccaccagcggcgacgtggtggtggtggccaccgacgccctgatgaccggcttcaccggcgacttcgacagcgtgatcgactgcaacacctgcgtgacccagaccgtggacttcagcctggaccccaccttcaccatcgagaccatcaccctgccccaggacgccgtgagccgcacccagcgccgcggccgcaccggccgcggcaagcccggcatctaccgcttcgtggcccccggcgagcgccccagcggcatgttcgacagcagcgtgctgtgcgagtgctacgacgccggctgcgcctggtacgagctgacccccgccgagaccaccgtgcgcctgcgcgcctacatgaacacccccggcctgcccgtgtgccaggaccacctggagttctgggagggcgtgttcaccggcctgacccacatcgacgcccacttcctgagccagaccaagcagagcggcgagaacctgccctacctggtggcctaccaggccaccgtgtgcgcccgcgcccaggcccccccccccagctgggaccagatgtggaagtgcctgatccgcctgaagcccaccctgcacggccccacccccctgctgtaccgcctgggcgccgtgcagaacgaggtgaccctgacccaccccgtgaccaagtacatcatgacctgcatgagcgccgacctggaggtggtgaccagcacctgggtgctggtgggcggcgtgctggccgccctggccgcctactgcctgagcaccggctgcgtggtgatcgtgggccgcatcgtgctgagcggcaagcccgccatcatccccgaccgcgaggtgctgtaccgcgagttcgacgagatggaggagtgcagccagcacctgccctacatcgagcagggcatggacatcgacccctacaaggagttcggcgccaccgtggagctgctgagcttcctgcccagcgacttcttccccagcgtgcgcgacctgctggacaccgccagcgccctgtaccgcgaggccctggagagccccgagcactgcagcccccaccacaccgccctgcgccaggccatcctgtgctggggcgagctgatgaccctggccacctgggtgggcgtgaacctggaggaccccgccagccgcgacctggtggtgagctacgtgaacaccaacatgggcctgaagttccgccagctgctgtggttccacatcagctgcctgaccttcggccgcgagaccgtgatcgagtacctggtgagcttcggcgtgtggatccgcaccccccccgcctaccgcccccccaacgcccccatcctgagcaccctgcccgagaccaccgtggtgcgccgccgcggccgcagcccccgccgccgcacccccagcccccgccgccgccgcagccagagcccccgccgccgccgcagccagagccgcgagagccagtgctag

SEQ ID NO: 80 - protein, 933 aa, Artificial Sequence
  CONSTR-5 NS3/4A-HBcAg Fragments
MAPITAYAQQTRGLLGCIITSLTGRDKNQVEGEVQIVSTAAQTFLATCINGVCWTVYHGAGTRTIASPKGPVIQMYTNVDQDLVGWPAPQGARSLTPCTCGSSDLYLVTRHADVIPVRRRGDGRGSLLSPRPISYLKGSSGGPLLCPAGHAVGIFRAAVCTRGVAKAVDFIPVESLETTMRSPVFSDNSSPPAVPQSYQVAHLHAPTGSGKSTKVPAAYAAQGYKVLVLNPSVAATMGFGAYMSKAHGIDPNIRTGVRTITTGSPITYSTYGKFLADGGCSGGAYDIIICDECHSTDATSILGIGTVLDQAETAGARLTVLATATPPGSVTVPHPNIEEVALSTTGEIPFYGKAIPLEAIKGGRHLIFCHSKKKCDELAAKLVALGVNAVAYYRGLDVSVIPTSGDVVVVATDALMTGFTGDFDSVIDCNTCVTQTVDFSLDPTFTIETITLPQDAVSRTQRRGRTGRGKPGIYRFVAPGERPSGMFDSSVLCECYDAGCAWYELTPAETTVRLRAYMNTPGLPVCQDHLEFWEGVFTGLTHIDAHFLSQTKQSGENLPYLVAYQATVCARAQAPPPSWDQMWKCLIRLKPTLHGPTPLLYRLGAVQNEVTLTHPVTKYIMTCMSADLEVVTSTWVLVGGVLAALAAYCLSTGCVVIVGRIVLSGKPAIIPDREVLYREFDEMEECSQHLPYIEQGMDIDPYKEFGATVELLSFLPSDFFPSVRDLLDTASALYREALESSADLEVVTSTWVLVGGVLPEHCSPHHTALRQAILCWGELMTLATWVGVNLEDPASRDLVVSSADLEVVTSTWVLVGGVLYVNTNMGLKFRQLLWFHISCLTFGRETVIEYLVSFGVWIRTPPAYRPPNAPILSSADLEVVTSTWVLVGGVLTLPETTVVRRRGRSPRRRTPSPRRRRSQSPRRRRSQSRESQC

SEQ ID NO: 81 - DNA, 2802 bp, Artificial Sequence
  CONSTR-5 NS3/4A-HBcAg Fragments
atggcccccatcaccgcctacgcccagcagacccgcggcctgctgggctgcatcatcaccagcctgaccggccgcgacaagaaccaggtggagggcgaggtgcagatcgtgagcaccgccgcccagaccttcctggccacctgcatcaacggcgtgtgctggaccgtgtaccacggcgccggcacccgcaccatcgccagccccaagggccccgtgatccagatgtacaccaacgtggaccaggacctggtgggctggcccgccccccagggcgcccgcagcctgaccccctgcacctgcggcagcagcgacctgtacctggtgacccgccacgccgacgtgatccccgtgcgccgccgcggcgacggccgcggcagcctgctgagcccccgccccatcagctacctgaagggcagcagcggcggccccctgctgtgccccgccggccacgccgtgggcatcttccgcgccgccgtgtgcacccgcggcgtggccaaggccgtggacttcatccccgtggagagcctggagaccaccatgcgcagccccgtgttcagcgacaacagcagcccccccgccgtgccccagagctaccaggtggcccacctgcacgcccccaccggcagcggcaagagcaccaaggtgcccgccgcctacgccgcccagggctacaaggtgctggtgctgaaccccagcgtggccgccaccatgggcttcggcgcctacatgagcaaggcccacggcatcgaccccaacatccgcaccggcgtgcgcaccatcaccaccggcagccccatcacctacagcacctacggcaagttcctggccgacggcggctgcagcggcggcgcctacgacatcatcatctgcgacgagtgccacagcaccgacgccaccagcatcctgggcatcggcaccgtgctggaccaggccgagaccgccggcgcccgcctgaccgtgctggccaccgccaccccccccggcagcgtgaccgtgccccaccccaacatcgaggaggtggccctgagcaccaccggcgagatccccttctacggcaaggccatccccctggaggccatcaagggcggccgccacctgatcttctgccacagcaagaagaagtgcgacgagctggccgccaagctggtggccctgggcgtgaacgccgtggcctactaccgcggcctggacgtgagcgtgatccccaccagcggcgacgtggtggtggtggccaccgacgccctgatgaccggcttcaccggcgacttcgacagcgtgatcgactgcaacacctgcgtgacccagaccgtggacttcagcctggaccccaccttcaccatcgagaccatcaccctgccccaggacgccgtgagccgcacccagcgccgcggccgcaccggccgcggcaagcccggcatctaccgcttcgtggcccccggcgagcgccccagcggcatgttcgacagcagcgtgctgtgcgagtgctacgacgccggctgcgcctggtacgagctgacccccgccgagaccaccgtgcgcctgcgcgcctacatgaacacccccggcctgcccgtgtgccaggaccacctggagttctgggagggcgtgttcaccggcctgacccacatcgacgcccacttcctgagccagaccaagcagagcggcgagaacctgccctacctggtggcctaccaggccaccgtgtgcgcccgcgcccaggcccccccccccagctgggaccagatgtggaagtgcctgatccgcctgaagcccaccctgcacggccccacccccctgctgtaccgcctgggcgccgtgcagaacgaggtgaccctgacccaccccgtgaccaagtacatcatgacctgcatgagcgccgacctggaggtggtgaccagcacctgggtgctggtgggcggcgtgctggccgccctggccgcctactgcctgagcaccggctgcgtggtgatcgtgggccgcatcgtgctgagcggcaagcccgccatcatccccgaccgcgaggtgctgtaccgcgagttcgacgagatggaggagtgcagccagcacctgccctacatcgagcagggcatggacatcgacccctacaaggagttcggcgccaccgtggagctgctgagcttcctgcccagcgacttcttccccagcgtgcgcgacctgctggacaccgccagcgccctgtaccgcgaggccctggagagcagcgccgacctggaggtggtgaccagcacctgggtgctggtgggcggcgtgctgcccgagcactgcagcccccaccacaccgccctgcgccaggccatcctgtgctggggcgagctgatgaccctggccacctgggtgggcgtgaacctggaggaccccgccagccgcgacctggtggtgagcagcgccgacctggaggtggtgaccagcacctgggtgctggtgggcggcgtgctgtacgtgaacaccaacatgggcctgaagttccgccagctgctgtggttccacatcagctgcctgaccttcggccgcgagaccgtgatcgagtacctggtgagcttcggcgtgtggatccgcaccccccccgcctaccgcccccccaacgcccccatcctgagcagcgccgacctggaggtggtgaccagcacctgggtgctggtgggcggcgtgctgaccctgcccgagaccaccgtggtgcgccgccgcggccgcagcccccgccgccgcacccccagcccccgccgccgccgcagccagagcccccgccgccgccgcagccagagccgcgagagccagtgctag

SEQ ID NO: 82 - protein, 933 aa, Artificial Sequence
  CONSTR-6 NS3/4A-HBcAg Fragments
MAPITAYAQQTRGLLGCIITSLTGRDKNQVEGEVQIVSTAAQTFLATCINGVCWTVYHGAGTRTIASPKGPVIQMYTNVDQDLVGWPAPQGARSLTPCTCGSSDLYLVTRHADVIPVRRRGDGRGSLLSPRPISYLKGSSGGPLLCPAGHAVGIFRAAVCTRGVAKAVDFIPVESLETTMRSPVFSDNSSPPAVPQSYQVAHLHAPTGSGKSTKVPAAYAAQGYKVLVLNPSVAATMGFGAYMSKAHGIDPNIRTGVRTITTGSPITYSTYGKFLADGGCSGGAYDIIICDECHSTDATSILGIGTVLDQAETAGARLTVLATATPPGSVTVPHPNIEEVALSTTGEIPFYGKAIPLEAIKGGRHLIFCHSKKKCDELAAKLVALGVNAVAYYRGLDVSVIPTSGDVVVVATDALMTGFTGDFDSVIDCNTCVTQTVDFSLDPTFTIETITLPQDAVSRTQRRGRTGRGKPGIYRFVAPGERPSGMFDSSVLCECYDAGCAWYELTPAETTVRLRAYMNTPGLPVCQDHLEFWEGVFTGLTHIDAHFLSQTKQSGENLPYLVAYQATVCARAQAPPPSWDQMWKCLIRLKPTLHGPTPLLYRLGAVQNEVTLTHPVTKYIMTCMSADLEVVTSTWVLVGGVLAALAAYCLSTGCVVIVGRIVLSGKPAIIPDREVLYREFDEMEECSQHLPYIEQGTLPETTVVRRRGRSPRRRTPSPRRRRSQSPRRRRSQSRESQCSADLEVVTSTWVLVGGVLPEHCSPHHTALRQAILCWGELMTLATWVGVNLEDPASRDLVVSSADLEVVTSTWVLVGGVLYVNTNMGLKFRQLLWFHISCLTFGRETVIEYLVSFGVWIRTPPAYRPPNAPILSSADLEVVTSTWVLVGGVLMDIDPYKEFGATVELLSFLPSDFFPSVRDLLDTASALYREALES

SEQ ID NO: 83 - DNA, 2802 bp, Artificial Sequence
  CONSTR-6 NS3/4A-HBcAg Fragments
atggcccccatcaccgcctacgcccagcagacccgcggcctgctgggctgcatcatcaccagcctgaccggccgcgacaagaaccaggtggagggcgaggtgcagatcgtgagcaccgccgcccagaccttcctggccacctgcatcaacggcgtgtgctggaccgtgtaccacggcgccggcacccgcaccatcgccagccccaagggccccgtgatccagatgtacaccaacgtggaccaggacctggtgggctggcccgccccccagggcgcccgcagcctgaccccctgcacctgcggcagcagcgacctgtacctggtgacccgccacgccgacgtgatccccgtgcgccgccgcggcgacggccgcggcagcctgctgagcccccgccccatcagctacctgaagggcagcagcggcggccccctgctgtgccccgccggccacgccgtgggcatcttccgcgccgccgtgtgcacccgcggcgtggccaaggccgtggacttcatccccgtggagagcctggagaccaccatgcgcagccccgtgttcagcgacaacagcagcccccccgccgtgccccagagctaccaggtggcccacctgcacgcccccaccggcagcggcaagagcaccaaggtgcccgccgcctacgccgcccagggctacaaggtgctggtgctgaaccccagcgtggccgccaccatgggcttcggcgcctacatgagcaaggcccacggcatcgaccccaacatccgcaccggcgtgcgcaccatcaccaccggcagccccatcacctacagcacctacggcaagttcctggccgacggcggctgcagcggcggcgcctacgacatcatcatctgcgacgagtgccacagcaccgacgccaccagcatcctgggcatcggcaccgtgctggaccaggccgagaccgccggcgcccgcctgaccgtgctggccaccgccaccccccccggcagcgtgaccgtgccccaccccaacatcgaggaggtggccctgagcaccaccggcgagatccccttctacggcaaggccatccccctggaggccatcaagggcggccgccacctgatcttctgccacagcaagaagaagtgcgacgagctggccgccaagctggtggccctgggcgtgaacgccgtggcctactaccgcggcctggacgtgagcgtgatccccaccagcggcgacgtggtggtggtggccaccgacgccctgatgaccggcttcaccggcgacttcgacagcgtgatcgactgcaacacctgcgtgacccagaccgtggacttcagcctggaccccaccttcaccatcgagaccatcaccctgccccaggacgccgtgagccgcacccagcgccgcggccgcaccggccgcggcaagcccggcatctaccgcttcgtggcccccggcgagcgccccagcggcatgttcgacagcagcgtgctgtgcgagtgctacgacgccggctgcgcctggtacgagctgacccccgccgagaccaccgtgcgcctgcgcgcctacatgaacacccccggcctgcccgtgtgccaggaccacctggagttctgggagggcgtgttcaccggcctgacccacatcgacgcccacttcctgagccagaccaagcagagcggcgagaacctgccctacctggtggcctaccaggccaccgtgtgcgcccgcgcccaggcccccccccccagctgggaccagatgtggaagtgcctgatccgcctgaagcccaccctgcacggccccacccccctgctgtaccgcctgggcgccgtgcagaacgaggtgaccctgacccaccccgtgaccaagtacatcatgacctgcatgagcgccgacctggaggtggtgaccagcacctgggtgctggtgggcggcgtgctggccgccctggccgcctactgcctgagcaccggctgcgtggtgatcgtgggccgcatcgtgctgagcggcaagcccgccatcatccccgaccgcgaggtgctgtaccgcgagttcgacgagatggaggagtgcagccagcacctgccctacatcgagcagggcaccctgcccgagaccaccgtggtgcgccgccgcggccgcagcccccgccgccgcacccccagcccccgccgccgccgcagccagagcccccgccgccgccgcagccagagccgcgagagccagtgcagcgccgacctggaggtggtgaccagcacctgggtgctggtgggcggcgtgctgcccgagcactgcagcccccaccacaccgccctgcgccaggccatcctgtgctggggcgagctgatgaccctggccacctgggtgggcgtgaacctggaggaccccgccagccgcgacctggtggtgagcagcgccgacctggaggtggtgaccagcacctgggtgctggtgggcggcgtgctgtacgtgaacaccaacatgggcctgaagttccgccagctgctgtggttccacatcagctgcctgaccttcggccgcgagaccgtgatcgagtacctggtgagcttcggcgtgtggatccgcaccccccccgcctaccgcccccccaacgcccccatcctgagcagcgccgacctggaggtggtgaccagcacctgggtgctggtgggcggcgtgctgatggacatcgacccctacaaggagttcggcgccaccgtggagctgctgagcttcctgcccagcgacttcttccccagcgtgcgcgacctgctggacaccgccagcgccctgtaccgcgaggccctggagagctag

SEQ ID NO: 84 - protein, 933 aa, Artificial Sequence
  CONSTR-7 NS3/4A-HBcAg Fragments
MAPITAYAQQTRGLLGCIITSLTGRDKNQVEGEVQIVSTAAQTFLATCINGVCWTVYHGAGTRTIASPKGPVIQMYTNVDQDLVGWPAPQGARSLTPCTCGSSDLYLVTRHADVIPVRRRGDGRGSLLSPRPISYLKGSSGGPLLCPAGHAVGIFRAAVCTRGVAKAVDFIPVESLETTMRSPVFSDNSSPPAVPQSYQVAHLHAPTGSGKSTKVPAAYAAQGYKVLVLNPSVAATMGFGAYMSKAHGIDPNIRTGVRTITTGSPITYSTYGKFLADGGCSGGAYDIIICDECHSTDATSILGIGTVLDQAETAGARLTVLATATPPGSVTVPHPNIEEVALSTTGEIPFYGKAIPLEAIKGGRHLIFCHSKKKCDELAAKLVALGVNAVAYYRGLDVSVIPTSGDVVVVATDALMTGFTGDFDSVIDCNTCVTQTVDFSLDPTFTIETITLPQDAVSRTQRRGRTGRGKPGIYRFVAPGERPSGMFDSSVLCECYDAGCAWYELTPAETTVRLRAYMNTPGLPVCQDHLEFWEGVFTGLTHIDAHFLSQTKQSGENLPYLVAYQATVCARAQAPPPSWDQMWKCLIRLKPTLHGPTPLLYRLGAVQNEVTLTHPVTKYIMTCMSADLEVVTSTWVLVGGVLAALAAYCLSTGCVVIVGRIVLSGKPAIIPDREVLYREFDEMEECSQHLPYIEQGTLPETTVVRRRGRSPRRRTPSPRRRRSQSPRRRRSQSRESQCSADLEVVTSTWVLVGGVLYVNTNMGLKFRQLLWFHISCLTFGRETVIEYLVSFGVWIRTPPAYRPPNAPILSSADLEVVTSTWVLVGGVLPEHCSPHHTALRQAILCWGELMTLATWVGVNLEDPASRDLVVSSADLEVVTSTWVLVGGVLMDIDPYKEFGATVELLSFLPSDFFPSVRDLLDTASALYREALES

SEQ ID NO: 85 - DNA, 2802 bp, Artificial Sequence
  CONSTR-7 NS3/4A-HBcAg Fragments
atggcccccatcaccgcctacgcccagcagacccgcggcctgctgggctgcatcatcaccagcctgaccggccgcgacaagaaccaggtggagggcgaggtgcagatcgtgagcaccgccgcccagaccttcctggccacctgcatcaacggcgtgtgctggaccgtgtaccacggcgccggcacccgcaccatcgccagccccaagggccccgtgatccagatgtacaccaacgtggaccaggacctggtgggctggcccgccccccagggcgcccgcagcctgaccccctgcacctgcggcagcagcgacctgtacctggtgacccgccacgccgacgtgatccccgtgcgccgccgcggcgacggccgcggcagcctgctgagcccccgccccatcagctacctgaagggcagcagcggcggccccctgctgtgccccgccggccacgccgtgggcatcttccgcgccgccgtgtgcacccgcggcgtggccaaggccgtggacttcatccccgtggagagcctggagaccaccatgcgcagccccgtgttcagcgacaacagcagcccccccgccgtgccccagagctaccaggtggcccacctgcacgcccccaccggcagcggcaagagcaccaaggtgcccgccgcctacgccgcccagggctacaaggtgctggtgctgaaccccagcgtggccgccaccatgggcttcggcgcctacatgagcaaggcccacggcatcgaccccaacatccgcaccggcgtgcgcaccatcaccaccggcagccccatcacctacagcacctacggcaagttcctggccgacggcggctgcagcggcggcgcctacgacatcatcatctgcgacgagtgccacagcaccgacgccaccagcatcctgggcatcggcaccgtgctggaccaggccgagaccgccggcgcccgcctgaccgtgctggccaccgccaccccccccggcagcgtgaccgtgccccaccccaacatcgaggaggtggccctgagcaccaccggcgagatccccttctacggcaaggccatccccctggaggccatcaagggcggccgccacctgatcttctgccacagcaagaagaagtgcgacgagctggccgccaagctggtggccctgggcgtgaacgccgtggcctactaccgcggcctggacgtgagcgtgatccccaccagcggcgacgtggtggtggtggccaccgacgccctgatgaccggcttcaccggcgacttcgacagcgtgatcgactgcaacacctgcgtgacccagaccgtggacttcagcctggaccccaccttcaccatcgagaccatcaccctgccccaggacgccgtgagccgcacccagcgccgcggccgcaccggccgcggcaagcccggcatctaccgcttcgtggcccccggcgagcgccccagcggcatgttcgacagcagcgtgctgtgcgagtgctacgacgccggctgcgcctggtacgagctgacccccgccgagaccaccgtgcgcctgcgcgcctacatgaacacccccggcctgcccgtgtgccaggaccacctggagttctgggagggcgtgttcaccggcctgacccacatcgacgcccacttcctgagccagaccaagcagagcggcgagaacctgccctacctggtggcctaccaggccaccgtgtgcgcccgcgcccaggcccccccccccagctgggaccagatgtggaagtgcctgatccgcctgaagcccaccctgcacggccccacccccctgctgtaccgcctgggcgccgtgcagaacgaggtgaccctgacccaccccgtgaccaagtacatcatgacctgcatgagcgccgacctggaggtggtgaccagcacctgggtgctggtgggcggcgtgctggccgccctggccgcctactgcctgagcaccggctgcgtggtgatcgtgggccgcatcgtgctgagcggcaagcccgccatcatccccgaccgcgaggtgctgtaccgcgagttcgacgagatggaggagtgcagccagcacctgccctacatcgagcagggcaccctgcccgagaccaccgtggtgcgccgccgcggccgcagcccccgccgccgcacccccagcccccgccgccgccgcagccagagcccccgccgccgccgcagccagagccgcgagagccagtgcagcgccgacctggaggtggtgaccagcacctgggtgctggtgggcggcgtgctgtacgtgaacaccaacatgggcctgaagttccgccagctgctgtggttccacatcagctgcctgaccttcggccgcgagaccgtgatcgagtacctggtgagcttcggcgtgtggatccgcaccccccccgcctaccgcccccccaacgcccccatcctgagcagcgccgacctggaggtggtgaccagcacctgggtgctggtgggcggcgtgctgcccgagcactgcagcccccaccacaccgccctgcgccaggccatcctgtgctggggcgagctgatgaccctggccacctgggtgggcgtgaacctggaggaccccgccagccgcgacctggtggtgagcagcgccgacctggaggtggtgaccagcacctgggtgctggtgggcggcgtgctgatggacatcgacccctacaaggagttcggcgccaccgtggagctgctgagcttcctgcccagcgacttcttccccagcgtgcgcgacctgctggacaccgccagcgccctgtaccgcgaggccctggagagctag

SEQ ID NO: 86 - protein, 933 aa, Artificial Sequence
  CONSTR-8 NS3/4A-HBcAg Fragments
MAPITAYAQQTRGLLGCIITSLTGRDKNQVEGEVQIVSTAAQTFLATCINGVCWTVYHGAGTRTIASPKGPVIQMYTNVDQDLVGWPAPQGARSLTPCTCGSSDLYLVTRHADVIPVRRRGDGRGSLLSPRPISYLKGSSGGPLLCPAGHAVGIFRAAVCTRGVAKAVDFIPVESLETTMRSPVFSDNSSPPAVPQSYQVAHLHAPTGSGKSTKVPAAYAAQGYKVLVLNPSVAATMGFGAYMSKAHGIDPNIRTGVRTITTGSPITYSTYGKFLADGGCSGGAYDIIICDECHSTDATSILGIGTVLDQAETAGARLTVLATATPPGSVTVPHPNIEEVALSTTGEIPFYGKAIPLEAIKGGRHLIFCHSKKKCDELAAKLVALGVNAVAYYRGLDVSVIPTSGDVVVVATDALMTGFTGDFDSVIDCNTCVTQTVDFSLDPTFTIETITLPQDAVSRTQRRGRTGRGKPGIYRFVAPGERPSGMFDSSVLCECYDAGCAWYELTPAETTVRLRAYMNTPGLPVCQDHLEFWEGVFTGLTHIDAHFLSQTKQSGENLPYLVAYQATVCARAQAPPPSWDQMWKCLIRLKPTLHGPTPLLYRLGAVQNEVTLTHPVTKYIMTCMSADLEVVTSTWVLVGGVLAALAAYCLSTGCVVIVGRIVLSGKPAIIPDREVLYREFDEMEECSQHLPYIEQGTLPETTVVRRRGRSPRRRTPSPRRRRSQSPRRRRSQSRESQCSADLEVVTSTWVLVGGVLYVNTNMGLKFRQLLWFHISCLTFGRETVIEYLVSFGVWIRTPPAYRPPNAPILSSADLEVVTSTWVLVGGVLMDIDPYKEFGATVELLSFLPSDFFPSVRDLLDTASALYREALESSADLEVVTSTWVLVGGVLPEHCSPHHTALRQAILCWGELMTLATWVGVNLEDPASRDLVVS

SEQ ID NO: 87 - DNA, 2802 bp, Artificial Sequence
  CONSTR-8 NS3/4A-HBcAg Fragments
atggcccccatcaccgcctacgcccagcagacccgcggcctgctgggctgcatcatcaccagcctgaccggccgcgacaagaaccaggtggagggcgaggtgcagatcgtgagcaccgccgcccagaccttcctggccacctgcatcaacggcgtgtgctggaccgtgtaccacggcgccggcacccgcaccatcgccagccccaagggccccgtgatccagatgtacaccaacgtggaccaggacctggtgggctggcccgccccccagggcgcccgcagcctgaccccctgcacctgcggcagcagcgacctgtacctggtgacccgccacgccgacgtgatccccgtgcgccgccgcggcgacggccgcggcagcctgctgagcccccgccccatcagctacctgaagggcagcagcggcggccccctgctgtgccccgccggccacgccgtgggcatcttccgcgccgccgtgtgcacccgcggcgtggccaaggccgtggacttcatccccgtggagagcctggagaccaccatgcgcagccccgtgttcagcgacaacagcagcccccccgccgtgccccagagctaccaggtggcccacctgcacgcccccaccggcagcggcaagagcaccaaggtgcccgccgcctacgccgcccagggctacaaggtgctggtgctgaaccccagcgtggccgccaccatgggcttcggcgcctacatgagcaaggcccacggcatcgaccccaacatccgcaccggcgtgcgcaccatcaccaccggcagccccatcacctacagcacctacggcaagttcctggccgacggcggctgcagcggcggcgcctacgacatcatcatctgcgacgagtgccacagcaccgacgccaccagcatcctgggcatcggcaccgtgctggaccaggccgagaccgccggcgcccgcctgaccgtgctggccaccgccaccccccccggcagcgtgaccgtgccccaccccaacatcgaggaggtggccctgagcaccaccggcgagatccccttctacggcaaggccatccccctggaggccatcaagggcggccgccacctgatcttctgccacagcaagaagaagtgcgacgagctggccgccaagctggtggccctgggcgtgaacgccgtggcctactaccgcggcctggacgtgagcgtgatccccaccagcggcgacgtggtggtggtggccaccgacgccctgatgaccggcttcaccggcgacttcgacagcgtgatcgactgcaacacctgcgtgacccagaccgtggacttcagcctggaccccaccttcaccatcgagaccatcaccctgccccaggacgccgtgagccgcacccagcgccgcggccgcaccggccgcggcaagcccggcatctaccgcttcgtggcccccggcgagcgccccagcggcatgttcgacagcagcgtgctgtgcgagtgctacgacgccggctgcgcctggtacgagctgacccccgccgagaccaccgtgcgcctgcgcgcctacatgaacacccccggcctgcccgtgtgccaggaccacctggagttctgggagggcgtgttcaccggcctgacccacatcgacgcccacttcctgagccagaccaagcagagcggcgagaacctgccctacctggtggcctaccaggccaccgtgtgcgcccgcgcccaggcccccccccccagctgggaccagatgtggaagtgcctgatccgcctgaagcccaccctgcacggccccacccccctgctgtaccgcctgggcgccgtgcagaacgaggtgaccctgacccaccccgtgaccaagtacatcatgacctgcatgagcgccgacctggaggtggtgaccagcacctgggtgctggtgggcggcgtgctggccgccctggccgcctactgcctgagcaccggctgcgtggtgatcgtgggccgcatcgtgctgagcggcaagcccgccatcatccccgaccgcgaggtgctgtaccgcgagttcgacgagatggaggagtgcagccagcacctgccctacatcgagcagggcaccctgcccgagaccaccgtggtgcgccgccgcggccgcagcccccgccgccgcacccccagcccccgccgccgccgcagccagagcccccgccgccgccgcagccagagccgcgagagccagtgcagcgccgacctggaggtggtgaccagcacctgggtgctggtgggcggcgtgctgtacgtgaacaccaacatgggcctgaagttccgccagctgctgtggttccacatcagctgcctgaccttcggccgcgagaccgtgatcgagtacctggtgagcttcggcgtgtggatccgcaccccccccgcctaccgcccccccaacgcccccatcctgagcagcgccgacctggaggtggtgaccagcacctgggtgctggtgggcggcgtgctgatggacatcgacccctacaaggagttcggcgccaccgtggagctgctgagcttcctgcccagcgacttcttccccagcgtgcgcgacctgctggacaccgccagcgccctgtaccgcgaggccctggagagcagcgccgacctggaggtggtgaccagcacctgggtgctggtgggcggcgtgctgcccgagcactgcagcccccaccacaccgccctgcgccaggccatcctgtgctggggcgagctgatgaccctggccacctgggtgggcgtgaacctggaggaccccgccagccgcgacctggtggtgagctag

SEQ ID NO: 88 - protein, 933 aa, Artificial Sequence
  CONSTR-9 NS3/4A-HBcAg Fragments
MAPITAYAQQTRGLLGCIITSLTGRDKNQVEGEVQIVSTAAQTFLATCINGVCWTVYHGAGTRTIASPKGPVIQMYTNVDQDLVGWPAPQGARSLTPCTCGSSDLYLVTRHADVIPVRRRGDGRGSLLSPRPISYLKGSSGGPLLCPAGHAVGIFRAAVCTRGVAKAVDFIPVESLETTMRSPVFSDNSSPPAVPQSYQVAHLHAPTGSGKSTKVPAAYAAQGYKVLVLNPSVAATMGFGAYMSKAHGIDPNIRTGVRTITTGSPITYSTYGKFLADGGCSGGAYDIIICDECHSTDATSILGIGTVLDQAETAGARLTVLATATPPGSVTVPHPNIEEVALSTTGEIPFYGKAIPLEAIKGGRHLIFCHSKKKCDELAAKLVALGVNAVAYYRGLDVSVIPTSGDVVVVATDALMTGFTGDFDSVIDCNTCVTQTVDFSLDPTFTIETITLPQDAVSRTQRRGRTGRGKPGIYRFVAPGERPSGMFDSSVLCECYDAGCAWYELTPAETTVRLRAYMNTPGLPVCQDHLEFWEGVFTGLTHIDAHFLSQTKQSGENLPYLVAYQATVCARAQAPPPSWDQMWKCLIRLKPTLHGPTPLLYRLGAVQNEVTLTHPVTKYIMTCMSADLEVVTSTWVLVGGVLAALAAYCLSTGCVVIVGRIVLSGKPAIIPDREVLYREFDEMEECSQHLPYIEQGYVNTNMGLKFRQLLWFHISCLTFGRETVIEYLVSFGVWIRTPPAYRPPNAPILSSADLEVVTSTWVLVGGVLTLPETTVVRRRGRSPRRRTPSPRRRRSQSPRRRRSQSRESQCSADLEVVTSTWVLVGGVLMDIDPYKEFGATVELLSFLPSDFFPSVRDLLDTASALYREALESSADLEVVTSTWVLVGGVLPEHCSPHHTALRQAILCWGELMTLATWVGVNLEDPASRDLVVS

SEQ ID NO: 89 - DNA, 2802 bp, Artificial Sequence
  CONSTR-9 NS3/4A-HBcAg Fragments
atggcccccatcaccgcctacgcccagcagacccgcggcctgctgggctgcatcatcaccagcctgaccggccgcgacaagaaccaggtggagggcgaggtgcagatcgtgagcaccgccgcccagaccttcctggccacctgcatcaacggcgtgtgctggaccgtgtaccacggcgccggcacccgcaccatcgccagccccaagggccccgtgatccagatgtacaccaacgtggaccaggacctggtgggctggcccgccccccagggcgcccgcagcctgaccccctgcacctgcggcagcagcgacctgtacctggtgacccgccacgccgacgtgatccccgtgcgccgccgcggcgacggccgcggcagcctgctgagcccccgccccatcagctacctgaagggcagcagcggcggccccctgctgtgccccgccggccacgccgtgggcatcttccgcgccgccgtgtgcacccgcggcgtggccaaggccgtggacttcatccccgtggagagcctggagaccaccatgcgcagccccgtgttcagcgacaacagcagcccccccgccgtgccccagagctaccaggtggcccacctgcacgcccccaccggcagcggcaagagcaccaaggtgcccgccgcctacgccgcccagggctacaaggtgctggtgctgaaccccagcgtggccgccaccatgggcttcggcgcctacatgagcaaggcccacggcatcgaccccaacatccgcaccggcgtgcgcaccatcaccaccggcagccccatcacctacagcacctacggcaagttcctggccgacggcggctgcagcggcggcgcctacgacatcatcatctgcgacgagtgccacagcaccgacgccaccagcatcctgggcatcggcaccgtgctggaccaggccgagaccgccggcgcccgcctgaccgtgctggccaccgccaccccccccggcagcgtgaccgtgccccaccccaacatcgaggaggtggccctgagcaccaccggcgagatccccttctacggcaaggccatccccctggaggccatcaagggcggccgccacctgatcttctgccacagcaagaagaagtgcgacgagctggccgccaagctggtggccctgggcgtgaacgccgtggcctactaccgcggcctggacgtgagcgtgatccccaccagcggcgacgtggtggtggtggccaccgacgccctgatgaccggcttcaccggcgacttcgacagcgtgatcgactgcaacacctgcgtgacccagaccgtggacttcagcctggaccccaccttcaccatcgagaccatcaccctgccccaggacgccgtgagccgcacccagcgccgcggccgcaccggccgcggcaagcccggcatctaccgcttcgtggcccccggcgagcgccccagcggcatgttcgacagcagcgtgctgtgcgagtgctacgacgccggctgcgcctggtacgagctgacccccgccgagaccaccgtgcgcctgcgcgcctacatgaacacccccggcctgcccgtgtgccaggaccacctggagttctgggagggcgtgttcaccggcctgacccacatcgacgcccacttcctgagccagaccaagcagagcggcgagaacctgccctacctggtggcctaccaggccaccgtgtgcgcccgcgcccaggcccccccccccagctgggaccagatgtggaagtgcctgatccgcctgaagcccaccctgcacggccccacccccctgctgtaccgcctgggcgccgtgcagaacgaggtgaccctgacccaccccgtgaccaagtacatcatgacctgcatgagcgccgacctggaggtggtgaccagcacctgggtgctggtgggcggcgtgctggccgccctggccgcctactgcctgagcaccggctgcgtggtgatcgtgggccgcatcgtgctgagcggcaagcccgccatcatccccgaccgcgaggtgctgtaccgcgagttcgacgagatggaggagtgcagccagcacctgccctacatcgagcagggctacgtgaacaccaacatgggcctgaagttccgccagctgctgtggttccacatcagctgcctgaccttcggccgcgagaccgtgatcgagtacctggtgagcttcggcgtgtggatccgcaccccccccgcctaccgcccccccaacgcccccatcctgagcagcgccgacctggaggtggtgaccagcacctgggtgctggtgggcggcgtgctgaccctgcccgagaccaccgtggtgcgccgccgcggccgcagcccccgccgccgcacccccagcccccgccgccgccgcagccagagcccccgccgccgccgcagccagagccgcgagagccagtgcagcgccgacctggaggtggtgaccagcacctgggtgctggtgggcggcgtgctgatggacatcgacccctacaaggagttcggcgccaccgtggagctgctgagcttcctgcccagcgacttcttccccagcgtgcgcgacctgctggacaccgccagcgccctgtaccgcgaggccctggagagcagcgccgacctggaggtggtgaccagcacctgggtgctggtgggcggcgtgctgcccgagcactgcagcccccaccacaccgccctgcgccaggccatcctgtgctggggcgagctgatgaccctggccacctgggtgggcgtgaacctggaggaccccgccagccgcgacctggtggtgagctag

SEQ ID NO: 90 - protein, 10 aa, Artificial Sequence
  TP-5 Restricted Sequence
GLLGCIITSL

SEQ ID NO: 91 - protein, 10 aa, Artificial Sequence
  TP-6 Restricted Sequence
TGSPITYSTY

SEQ ID NO: 92 - protein, 9 aa, Artificial Sequence
  TP-7 Restricted Sequence
KLVAGVNAV

SEQ ID NO: 93 - protein, 9 aa, Artificial Sequence
  TP-9 Restricted Sequence
CINGVCWTV

SEQ ID NO: 94 - protein, 9 aa, Artificial Sequence
  TP-10 Restricted Sequence
LLCPAGHAV

SEQ ID NO: 95 - protein, 9 aa, Artificial Sequence
  TP-11 Restricted Sequence
ATMGFGAYM

SEQ ID NO: 96 - protein, 9 aa, Artificial Sequence
  TP-12 Restricted Sequence
YLVAYQATV

SEQ ID NO: 97 - protein, 9 aa, Artificial Sequence
  TP-13 Restricted Sequence
TLHGPTPLL

SEQ ID NO: 98 - DNA, 30 bp, Artificial Sequence
  NS4KR Primer
ccgtctagatcagcactcttccatttcatc

SEQ ID NO: 99 - DNA, 30 bp, Artificial Sequence
  NS3KF primer
cctgaattcatggcgcctatcacggcctat

SEQ ID NO: 100 - DNA, 2061 bp, HCV
  misc_feature (0)...(0): Hepatitis C virus NS3/4A coding region
atggcgcctatcacggcctatgcccagcagacaaggggccttttgggatgcataatcaccagcttgaccggccgggacaaaaaccaggtggagggtgaggttcagatcgtgtcaactgctgcccagactttcttggcaacctgcattaacggggtgtgttggactgtctaccatggagccggaacaaggaccattgcgtcacctaagggtcctgttatccagatgtacaccaatgtggaccaagacctcgtaggctggcccgctccccaaggtgcccgctcattaacaccatgcacttgcggctcctcggacctttacctggtcacgaggcacgccgatgtcattcctgtgcgccgacggggtgatggcaggggcagcctgctttcgccccggcctatctcttacttgaaaggctcctcgggaggccctctgctgtgccccgcaggacatgccgtaggcatattcagagccgcggtatgcacccgtggagtggctaaggcggtggacttcatccccgtagagagcttagagacaaccatgaggtccccggtgttctcagacaactcctccccaccagcagtgccccagagctaccaagtggcccacctgcatgctcccaccggcagcggtaagagcaccaaggtcccggccgcatacgcagctcagggctacaaggtgctggtgctcaacccctccgttgctgcaacaatgggctttggtgcttacatgtccaaggcccatgggattgatcctaacatcaggactggggtgaggacaattactactggcagcccgatcacgtattccacctacggcaagttccttgccgacggcgggtgttcagggggtgcttatgacataataatttgtgacgagtgccactccacggatgcaacatccatcttgggcattggcactgtccttgaccaagcagagaccgcgggggcgagactgactgtgctcgccaccgctacccctccgggctccgtcactgtgccccatcctaacatcgaggaggttgctctgtccactaccggagagatccccttttatggcaaggctattccccttgaagcaattaagggggggagacatctcatcttctgccactcaaagaagaagtgcgacgagctcgccgcaaaactggtcgcgttgggcgtcaatgccgtggcttactaccgcggccttgatgtgtccgtcatcccgaccagtggtgacgttgtcgtcgtggcaactgacgccctcatgaccggctttaccggcgacttcgattcggtgatagactgcaacacgtgtgtcacccagacagtcgacttcagccttgaccctaccttcaccattgagacaatcacgcttccccaggatgctgtctcccgtactcaacgtcggggtaggactggcagagggaagccaggcatctacagatttgtggcaccgggggagcgtccttctggcatgtttgactcgtctgtcctctgcgagtgctatgacgcgggttgtgcttggtatgagcttacgcccgccgagaccacagttaggctacgagcatacatgaacaccccgggacttcccgtgtgccaagaccatcttgaattttgggagggcgtctttacgggtctcacccacatagacgcccacttcctatcccagacaaagcagagtggggaaaaccttccctatctggtagcgtaccaagccaccgtgtgcgctagagctcaagcccctcccccgtcgtgggaccagatgtggaagtgcttgatccgtctcaagcccaccctccatgggccaacacctctgctatatagactgggcgctgtccagaatgaagtcaccctgacgcacccagtcaccaagtatatcatgacatgtatgtcggctgacctggaggtcgtcacgagtacctgggtgctcgttggcggcgttctggctgctttggccgcgtattgcctatccacaggctgcgtggtcatagtaggtaggattgtcttgtccggaaagccggcaatcatacccgacagggaagtcctctaccgggagttcgatgaaatggaagagtgctga

SEQ ID NO: 101 - DNA, 24 bp, Artificial Sequence
  NS3KF Primer
ccacgcggccgcgacgacctacag

SEQ ID NO: 102 - protein, 262 aa, Artificial Sequence; identical to SEQ ID NO: 19
  c.o. Stork HBcAg w/ human HBcAg insert (87-129)

SEQ ID NO: 103 - DNA, 789 bp, Artificial Sequence; identical to SEQ ID NO: 20
  c.o. Stork HBcAg w/ human HBcAg insert (87-129)

SEQ ID NO: 104 - protein, 262 aa, Artificial Sequence; identical to SEQ ID NO: 21
  c.o. Heron HBcAg w/ human HBcAg insert (87-129)

SEQ ID NO: 105 - DNA, 789 bp, Artificial Sequence; identical to SEQ ID NO: 22
  c.o. Heron HBcAg w/ human HBcAg insert (87-129)

SEQ ID NO: 106 - DNA, 9 bp, Artificial Sequence
  Kozak Sequence
gcaccatgg

(19) United States
(12) Patent Application Publication
(10) Pub. No.: US 2014/0286985 A1
(43) Pub. Date: Sep. 25, 2014

(54) SIMPLE VACCINES FROM DNA LAUNCHED SUICIDAL FLAVIVIRUSES

(71) Applicant: ChronTech Pharma AB, Huddinge (SE)

(72) Inventors: Matti Sallberg, Stockholm (SE); Lars Frelin, Alvsjo (SE); Magnus Johansson, Stockholm (SE)

(73) Assignee: ChronTech Pharma AB, Huddinge (SE)

(21) Appl. No.: 14/221,662

(22) Filed: Mar. 21, 2014

Related U.S. Application Data
(63) Continuation of application No. 13/104,909, filed on May 10, 2011, now abandoned.
(60) Provisional application No. 61/333,705, filed on May 11, 2010.

(51) Int. Cl. C07K 14/005

(52) U.S. Cl. CPC C07K 14/005; C12N 2770/28134
USPC 424/189.1

(57) ABSTRACT

Immunogenic compositions relating to DNA launched suicidal flaviviruses and methods of administering the same are described herein.

### CROSS-REFERENCE TO RELATED APPLICATIONS

This application is a continuation of U.S. patent application Ser. No. 13/104,909, filed May 10, 2011, which claims the benefit under 35 U.S.C. §119(e) of U.S. Provisional Application No. 61/333,705, filed May 11, 2010, the entirety of which is hereby expressly incorporated by reference.

### REFERENCE TO SEQUENCE LISTING

The present application is being filed along with a Sequence Listing in electronic format. The Sequence Listing is provided as a file entitled TRIPEP119C1_SEQLISTING.TXT, created Mar. 20, 2014, which is 364 KB in size. The information in the electronic format of the Sequence Listing is incorporated herein by reference in its entirety.

### BACKGROUND

Traditionally, vaccines have been based on live attenuated or inactivated pathogens. These strategies are inefficient, however, largely because of the antigenic variability of pathogens (e.g., viruses). Several peptide vaccines that comprise antigenic peptides or peptide fragments of pathogens have been developed. Conserved peptide fragments are less likely to exhibit antigenic variability and can overcome some of the problems associated with traditional peptides. Accordingly, subunit vaccines have been developed, which target conserved regions of pathogens. Synthetic peptide vaccines tend to be poorly immunogenic, however. The poor immunogenicity of synthetic peptide vaccines may be attributed to the fact that although these types of vaccines induce humoral antibody responses, they are less likely to induce cell-mediated responses.

Several investigators have sought to improve the antigenicity of synthetic peptide vaccines. For example, Klein et al. describe the engineering of chimeric proteins that comprise an immunogenic region of a protein from a first antigen linked to an immunogenic region from a second pathogen. (See, U.S. Pat. Nos. 6,033,668; 6,017,539; 5,998,169; and 5,968,776). Others have sought to create chimeric proteins that couple B-cell epitopes to universal T-cell epitopes in order to improve the immune response. (See, e.g., U.S. Pat. No. 5,114,713). Russell-Jones et al. (U.S. Pat. No. 5,928,644) also disclose T-cell epitopes derived from the TraT protein of Escherichia coli, which are used to produce hybrid molecules so as to generate an immune response to parasites, soluble factors (e.g., LSH) and viruses. Further, Ruslan (U.S. Patent Application Publication No. 20030232055) discloses the manufacture of vaccines based on PAMPs and immunogenic antigens.

The hepatitis B virus core antigen (HBcAg) is thought to be a key target for the host immune response in the control of the infection. In particular, the presence of HBcAg-specific T cells has been associated with clearance of acute and chronic infections with the hepatitis B virus (HBV). Subsequently, prophylactic and therapeutic vaccines that induce HBcAg-specific T cells have been developed and some have shown efficacy in infectious models. However, despite the high immunogenicity of exogenous HBcAg, many of the studies using endogenous HBcAg as a vaccine have been disappointing.

When expressed alone, HBcAg will spontaneously assemble into virus-like particles (VLPs) that are immunogenic in vivo. These VLPs interact with B cells as the primary antigen-presenting cell (APC) by an unusual interaction with the B cell receptor. HBcAg effectively primes specific T helper (Th) and, much less effectively, cytotoxic T cells (CTLs) as an exogenous antigen when high antigen doses in adjuvant are used. Both DNA and retrovirus-based immunizations using HBcAg have been reported to induce detectable HBcAg-specific CTLs in mice. Some investigators have sought to use HBcAg VLPs as a platform to display heterogeneous antigens, as well, but these approaches have been hindered by poor assembly and instability of the particles. (See e.g., U.S. Pat. Nos. 4,818,527; 4,882,145; 5,143,726; 6,231,864; 6,887,464; 6,942,866; 7,144,712; 7,320,795; 7,351,413; and 7,361,352; the disclosures of which are hereby expressly incorporated by reference in their entireties).

DNA vaccines can be used as a model to study the endogenous immunogenicity of antigens. However, phase I/II clinical trials reveal that it is difficult to prime robust immune responses in humans with direct intramuscular injections of DNA vaccines. Different modes of DNA delivery have now become available, including transdermal delivery of DNA coated to gold beads using the gene gun or treatment of the injection site by in vivo electroporation. The need for approaches that enhance the immune response of a subject after vaccination, in particular DNA vaccination, is manifest.

### SUMMARY OF THE INVENTION

Several embodiments provided herein relate to immunogenic compositions including: (a) a first construct that comprises a nucleic acid sequence encoding a tick-borne encephalitis (TBE) core, Pre-M, and envelope proteins, but lacking the TBE non-structural replicon proteins; and (b) a second construct that comprises a nucleic acid sequence encoding a hepatitis C virus (HCV) NS3/4A fusion protein and TBE non-structural replicon proteins.

In one aspect, the nucleic acid encoding the NS3/4A fusion protein comprises a nucleic acid sequence of SEQ ID NO: 2. In another aspect, the second construct further comprises a 5′ untranslated nucleic acid sequence, a nucleic acid sequence encoding an internal ribosome entry site (IRES) element 5′ to the NS3/4A fusion protein and TBE non-structural replicon proteins, and a 3′ untranslated nucleic acid sequence. In a further aspect, the second construct further comprises a nucleic acid sequence encoding a HCV NS5A protein.

In another aspect, the aforementioned embodiments further include a third construct that comprises a nucleic acid sequence encoding a hepatitis B core antigen (HBcAg) and TBE non-structural replicon proteins. In one aspect, the third construct further includes a 5′ untranslated nucleic acid sequence, a nucleic acid sequence encoding an IRES element 5′ to the HBcAg and TBE non-structural replicon proteins, and a 3′ untranslated nucleic acid sequence. In a further aspect, the HBcAg is stork or heron HBcAg. In the same aspect, the stork or heron HBcAg comprises the nucleic acid sequence of SEQ ID NO: 20 or SEQ ID NO: 22, respectively.

In an additional aspect of the aforementioned embodiments, the first construct further includes a constitutive promoter operably linked to the nucleic acid sequence encoding the TBE core, Pre-M, and envelope proteins. In one aspect, the first and second constructs are capable of generating TBE particles that can infect once and produce new non-structural replicon proteins and the NS3/4A fusion protein in a subject administered the immunogenic composition.

Several embodiments provided herein relate to immunogenic compositions including: (a) a first construct that comprises a nucleic acid sequence encoding a tick-borne encephalitis (TBE) core, Pre-M, and envelope proteins, but lacking the TBE non-structural replicon proteins; (b) a second construct that comprises a 5′ untranslated nucleic acid sequence, a nucleic acid sequence encoding an IRES element, a nucleic acid sequence encoding a hepatitis C virus (HCV) NS3/4A fusion protein and TBE non-structural replicon proteins, and a 3′ untranslated nucleic acid sequence; and (c) a third construct that comprises a 5′ untranslated nucleic acid sequence, a nucleic acid sequence encoding an IRES element, a nucleic acid sequence encoding a hepatitis B core antigen (HBcAg) and TBE non-structural replicon proteins, and a 3′ untranslated nucleic acid sequence. In one aspect, the HBcAg is stork or heron HBcAg. In the same aspect, the stork or heron HBcAg comprises the nucleic acid sequence of SEQ ID NO: 20 or SEQ ID NO: 22, respectively.

Several embodiments provided herein relate to methods of generating an immune response in a subject including: providing a first construct that comprises a nucleic acid sequence encoding a tick-borne encephalitis (TBE) core, Pre-M, and envelope proteins, but lacking the TBE non-structural replicon proteins; providing a second construct that comprises a nucleic acid sequence encoding a hepatitis C virus (HCV) NS3/4A fusion protein and TBE non-structural replicon proteins; and administering the first and second constructs to the subject.

In one aspect, the methods further include administering a third construct that comprises a nucleic acid sequence encoding a hepatitis B core antigen (HBcAg) and TBE non-structural replicon proteins, wherein the third construct enhances the immune response to the NS3/4A fusion protein. In another aspect, the first and second constructs are coadministered to the subject. In the same aspect, the coadministration is performed by intramuscular injection.

In a further aspect of the aforementioned embodiments, any one of the first, second, or third constructs is administered separately from the other two constructs. In another aspect, the HBcAg is stork or heron HBcAg. In an additional aspect, the subject has been identified as having an HCV or HBV infection.

### BRIEF DESCRIPTION OF THE DRAWINGS

FIGS. 1 (a-i) illustrate constructs encoding HBcAg and HCV NS3/N4A.

FIGS. 2 (a-b) show the results from transcription and translation assays on nucleic acids that encode HBcAg and HCV NS3/N4A. The products were separated by gel electrophoresis.

FIGS. 3 (a-e) show the results from an ELISpot assay conducted on HLA-A2 transgenic mice, which had been administered nucleic acids that encode HBcAg and HCV NS3/N4A.

FIGS. 4 (a-e) show the results of an ELISpot assay conducted on HCV NS3/4A+HLA-A2 transgenic mice, which had been administered nucleic acids that encode HBcAg and HCV NS3/N4A.

FIG. 5 is a schematic illustration of a plasmid (A) that encodes structural proteins that form a tick-borne encephalitis (TBE) virus-like particle (VLP), a plasmid (B) that encodes HCV NS3/4A antigen and TBE non-structural proteins, and a plasmid (C) that encodes HBcAg antigen and TBE non-structural proteins.

FIG. 6 is a schematic illustration of a method of vaccinating a subject with a first plasmid that forms a flavivirus VLP and a second plasmid that encodes the flavivirus replicon and gene-of-interest immunogen.

FIG. 7 is a schematic illustration of a method of vaccinating a subject with a first plasmid that forms a flavivirus VLP and a second plasmid that encodes the flavivirus replicon and gene-of-interest immunogen.

### DETAILED DESCRIPTION

It has been discovered that hepatitis B core antigen (HBcAg) is a potent adjuvant that improves the immune response of a subject to a co-administered antigen. Disclosed herein are the results of experiments that revealed that a nucleic acid encoding HBcAg improved the immune response of a mammal to a co-administered nucleic acid encoding a hepatitis C virus (HCV) protein (NS3/4A). Accordingly, some embodiments include methods enhancing or improving an immune response of a subject, wherein an HBcAg or a nucleic acid encoding an HBcAg is provided to a subject in a mixture with a peptide immunogen or a nucleic acid encoding a peptide immunogen. In some embodiments, the peptide immunogen or nucleic acid encoding a peptide immunogen is provided in Cis with the HBcAg (e.g., a fusion protein encoding HBcAg joined to a desired peptide antigen or a nucleic acid encoding said fusion protein, see for example SEQ. ID. Nos 24, 26, 28, 30, 32, 34, 36, 38, 40, 42, 44, 46, 48, 50, 51, 52, 54, 56, 58, 60, 62, 64, 66, 68 and 73, 75, 77, 79, 81, 83, 85, 87 and 89). In other embodiments, the peptide immunogen or nucleic acid encoding a peptide immunogen is provided in Trans with the HBcAg (e.g., HBcAg or a nucleic acid encoding HBcAg (e.g., SEQ. ID. NO. 10, 20 and 22) is provided in a mixture or co-administered with a desired peptide antigen or a nucleic acid encoding said desired peptide antigen (e.g., SEQ. ID. NOs. 2, 8, 10, 12, 14, 16, and 18). Preferably, the compositions described herein comprise, consist essentially of, or consist of an “avian HBcAg,” that is an HBcAg derived from a hepatitis virus that infects a bird, such as stork or heron). It is contemplated that the use of avian HBcAg in the compositions described herein will allow the formulation of immunogenic compositions that are suitable for administration to HBV infected individuals or subjects that have antibodies specific for HBV, since antibodies specific for an HBV that infects humans (“human HBV”) generally do not cross-react with the HBV that infects avian species, such as stork and heron. Additionally, it is preferred that the nucleic acid sequences used in the compositions and methods disclosed herein are codon-optimized for expression in the subject to which the immunogenic compositions are to be administered (e.g., humans).

Accordingly, one or more of the compositions described herein can be used to improve, enhance or generate an immune response in a subject. By some approaches, a subject in need of an immune response to a particular antigen is identified. The identification step can be accomplished by diagnostic approaches or clinical evaluation (e.g., a subject in need of an immune response to HCV can be identified by diagnostic test or clinical evaluation). Next, one or more of the HBcAg-containing compositions described herein is provided to the identified subject. In some embodiments, the composition comprises an HBcAg protein or fragment thereof that is at least, equal to or any number in between about 20, 30, 40, 50, 60, 70, 80, 90, 100, 150, or more amino acids (e.g., HBcAg from a hepatitis that infects birds or humans) and an antigen to which an immune response is desired (e.g., an HCV protein, such as NS3/4A or NS5A). In other embodiments, the composition comprises a nucleic acid that encodes an HBcAg protein or a fragment thereof that is at least, equal to, or any number in between about 20, 30, 40, 50, 60, 70, 80, 90, 100, 200, 250, 300, or more amino acids (e.g., HBcAg from a hepatitis that infects birds or humans) and a nucleic acid encoding an antigen to which an immune response is desired (e.g., an HCV protein, such as NS3/4A or NS5A). In more embodiments, the composition comprises an HBcAg protein or fragment thereof that is at least, equal to or any number in between about 20, 30, 40, 50, 60, 70, 80, 90, 100, 150 or more amino acids (e.g., HBcAg from a hepatitis that infects birds or humans) and a nucleic acid encoding an antigen to which an immune response is desired (e.g., an HCV protein, such as NS3/4A or NS5A). In still more embodiments, the composition comprises a nucleic acid that encodes an HBcAg protein or a fragment thereof that is at least, equal to, or any number in between about 20, 30, 40, 50, 60, 70, 80, 90, 100, 150, or more amino acids (e.g., HBcAg from a hepatitis that infects birds or humans) and an antigen to which an immune response is desired (e.g., an HCV protein, such as NS3/4A or NS5A). Preferably, the compositions described above utilize an HBcAg protein or nucleic acid encoding an HBcAg protein that is derived from an avian hepatitis virus, such as stork or heron (e.g., SEQ. ID. NO. 20 and 22). Preferably, the peptide antigens or nucleic acids encoding said peptide antigens are hepatitis antigens, such as HCV antigens (e.g., NS3/4A or NS5A), HBV antigens (e.g., HBV surface antigen, HBV e antigen, human HBcAg, a human HBV polymerase antigen, a human HBV x antigen) or said peptide antigens or nucleic acids encoding said peptide antigens are birch allergens. Exemplary constructs and nucleic acids encoding preferred antigens, which can be used in one or more of the compositions and methods described herein are provided in SEQ. ID. NOs. 2, 8, 10, 12, 14, 16, and 18. Optionally, any of the aforementioned approaches can further include the step of measuring the immune response of the subject before, during, and after administration of the immunogenic composition. Such measurements can be made, for example, by diagnostic evaluation of viral titer in the case of viral disease, clinical evaluation, and scratch tests as are used when evaluating the response to allergens.

Generally, the generation, enhancement, or improvement of an immune response refers to an induction of a humoral (antibody) response and/or a cellular response. Most simply, an increase in the amount of antigen-specific antibodies (e.g., total IgG) can be seen by utilizing one or more of the embodiments described herein. Enhancement of an immune response also refers to any statistically significant change in the level of one or more immune cells (T cells, B cells, antigen-presenting cells, dendritic cells and the like) or in the activity of one or more of these immune cells (cytotoxic T lymphocyte (CTL) activity, helper T lymphocyte (HTL) activity, cytokine secretion, change in profile of cytokine secretion). The skilled artisan will readily appreciate that several methods for measuring or establishing whether an immune response is generated, enhanced, or improved are available. A variety of methods for detecting the presence and levels of an immune response are available, for example. (See, e.g., Current Protocols in Immunology, Ed: John E. Coligan, et al. (2001) John Wiley & Sons, NY, N.Y.; Current Protocols in Molecular Biology, (2001), Greene Publ. Assoc. Inc. & John Wiley & Sons, NY, N.Y.; Ausubel et al. (2001) Current Protocols in Molecular Biology, Greene Publ. Assoc. Inc. & John Wiley & Sons, Inc., NY, N.Y.; Sambrook et al. (1989) Molecular Cloning, Second Ed., Cold Spring Harbor Laboratory, Plainview, N.Y.); Maniatis et al. (1982) Molecular Cloning, Cold Spring Harbor Laboratory, Plainview, N.Y.; and elsewhere). Illustrative methods useful in this context include intracellular cytokine staining (ICS), ELISPOT, proliferation assays, cytotoxic T cell assays including chromium release or equivalent assays, and gene expression analysis using any number of polymerase chain reaction (PCR) or RT-PCR based assays. For example, the number of CD8⁺ T-cells specific for a particular antigen or T-cell epitope (TCE) can be measured by flow cytometry. (See, e.g., Frelin et al. (2004) Gene Therapy 11:522-533). CTL priming can also be measured in vivo by, for example, a tumor inhibition model, in which the ability of an animal (e.g., mouse) to inhibit growth of tumors derived from tumor cells engineered to express the antigen of interest. Id.

In some embodiments, generation or enhancement of an immune response comprises an increase in target-specific CTL activity of between 1.5 and 5 fold in a subject that is provided a composition that comprises the nucleic acids or polypeptides disclosed herein (e.g., in the context of a HBcAg nucleic acid or polypeptide), wherein the TCE is derived from the target, as compared to the same TCE that is not provided in the context of the compositions disclosed herein. In some embodiments, an enhancement of an immune response comprises an increase in target-specific CTL activity of about 2, 2.5, 3, 3.5, 4, 4.5, 5, 5.5, 6, 6.5, 7, 7.5, 8, 8.5, 9, 9.5, 10, 10.5, 11, 11.5, 12, 12.5, 15, 16, 17, 18, 19, 20, or more fold in a subject that is provided a composition that comprises a nucleic acid or a polypeptide disclosed herein (e.g., in the context of a HBcAg nucleic acid or polypeptide), wherein the TCE is derived from the target, as compared to administration of the same TCE that is not provided in the context of the compositions disclosed herein.

In other embodiments, an alteration of an immune response comprises an increase in target-specific HTL activity, such as proliferation of helper T cells, of between 1.5 and 5 fold in a subject that is provided a composition that comprises a nucleic acid or polypeptide disclosed herein (e.g., in the context of a HBcAg nucleic acid or polypeptide), wherein the TCE is derived from the target, as compared to the same TCE that is not provided in the context of the compositions disclosed herein. In some embodiments, alteration of an immune response comprises an increase in target-specific HTL activity of about 2, 2.5, 3, 3.5, 4, 4.5, 5, 5.5, 6, 6.5, 7, 7.5, 8, 8.5, 9, 9.5, 10, 10.5, 11, 11.5, 12, 12.5, 15, 16, 17, 18, 19, 20, or more fold in a subject that is provided a composition that comprises a nucleic acid or polypeptide disclosed herein (e.g., in the context of a HBcAg nucleic acid or polypeptide), wherein the TCE is derived from the target, as compared to administration of the same TCE that is not provided in the context of the compositions disclosed herein. In this context, an enhancement in HTL activity may comprise an increase as described above, or decrease, in production of a particular cytokine, such as interferon-gamma (IFNγ), interleukin-1 (IL-1), IL-2, IL-3, IL-6, IL-7, IL-12, IL-15, tumor necrosis factor-alpha (TNFα), granulocyte macrophage colony-stimulating factor (GM-CSF), granulocyte-colony stimulating factor (G-CSF), or other cytokine. In this regard, generation or enhancement of an immune response may comprise a shift from a Th2 type response to a Th1 type response or in certain embodiments a shift from a Th1 type response, to a Th2 type response. In other embodiments, the generation or enhancement of an immune response may comprise the stimulation of a predominantly Th1 or a Th2 type response.

In still more embodiments, an increase in the amount of antibody specific for the antigen (e.g., total IgG) is increased. Some embodiments, for example, generate an increase in heterologous target-specific antibody production of between 1.5, 2, 3, 4, or 5 fold in a subject that is provided a composition comprising the nucleic acids or polypeptides disclosed herein, (e.g., in the context of a HBcAg nucleic acid or polypeptide), wherein the TCE is derived from the target, as compared to the same TCE that is not present in the context of the compositions disclosed herein. In some embodiments, the increase in heterologous target-specific antibody production is about 2, 2.5, 3, 3.5, 4, 4.5, 5, 5.5, 6, 6.5, 7, 7.5, 8, 8.5, 9, 9.5, 10, 10.5, 11, 11.5, 12, 12.5, 15, 16, 17, 18, 19, 20, or more fold in a subject that is provided a composition that comprises a nucleic acid or polypeptide disclosed herein, (e.g., in the context of a HBcAg nucleic acid or polypeptide), wherein the TCE is derived from the target, as compared to as compared to administration of the same TCE that is not present in the context of the compositions disclosed herein.

Generation or enhancement of a cellular immune response can also refer to the frequency of cytotoxic T lymphocytes (CTLs) specific for a desired antigen that are primed, or the rapidity of priming of cytotoxic T lymphocytes (CTLs) specific for a desired antigen, compared to the priming of CTLs specific for the desired epitope when the epitope is not presented in the context of the nucleic acids or peptides disclosed herein. The section below describes several of the HBcAg and heterologous protein sequences that can be used in the compositions and methods described herein.

Several embodiments described herein concern isolated nucleic acids, peptides, compositions and methods that are useful for the generation, enhancement, or improvement of an immune response to a target antigen. These compositions are particularly useful to enhance the immune response of a subject that receives a protein or nucleic acid-based immunogen (e.g., DNA immunogen or conventional protein-based vaccine). Although Hepatitis B virus core antigen (HBcAg) is a well known antigen, HBcAg or portions thereof have not been described for use as an adjuvant, which can be administered to a subject in conjunction with a protein or nucleic acid-based immunogen (e.g., a DNA vaccine) so as to improve the immune response to the protein or the protein encoded by the nucleic acid immunogen. In a first series of experiments disclosed herein, it was discovered that a nucleic acid encoding HBcAg improved the immune response of a subject to a co-administered nucleic acid encoding a hepatitis C virus (HCV) protein (NS3/4A).

In this disclosure, it is revealed that HBcAg, in particular non-human HBcAgs, such as those derived from an avian hepatitis virus, in particular, the virus that infects stork and heron, are uniquely suitable for enhancing an immune response of a subject to a co-administered antigen (e.g., a nucleic acid or peptide immunogen that is administered in a mixture with the HBcAg adjuvant or within approximately at least, equal to, or any number in between 1, 2, 3, 4, 5, 6, 7, 8, 9, 10, 11, 12, 13, 14, 15, 20, 30, 45, or 60 minutes before or after inoculation with the immunogen). It is contemplated that HBcAg and fragments thereof or a nucleic acid encoding these compositions are useful additions to immunogen preparations, which improve the immune response of a subject (e.g., a human or mammal) to the immunogen. Sequences described herein are provided in Annex A.

Preferably, an HBcAg derived from a hepatitis virus that does not infect a human (a “non-human HBcAg”) or a nucleic acid encoding said non-human HBcAg is used as the adjuvant (e.g., an HBcAg derived from an avian hepatitis virus, such as the hepatitis virus that infects stork or heron (e.g., SEQ. ID. NOs. 20 and 22). HBV now afflicts almost a third of the world's population. Accordingly, a significant amount of the population has antibodies that react to an HBcAg derived from a hepatitis virus that infects humans. By utilizing HBcAg sequences derived from divergent hepatitis species, the compositions described herein can be made suitable for introduction into subjects that are already infected with HBV or subjects that have already generated antibodies to HBV (e.g., a subject that had been previously inoculated with an HBV vaccine). Additionally, when nucleic acids encoding an HBcAg or a fragment thereof (e.g., a nucleic acid encoding an HBcAg derived from an avian hepatitis virus that infects stork or heron) are administered, these sequences are, preferably, codon-optimized for expression in the subject (e.g., codon-optimized for expression in the particular animal or human (e.g., SEQ. ID. NOs. 20 and 22)).

Accordingly, several aspects of the invention described herein concern compositions that comprise, consist essentially of, or that consist of nucleic acids that encode an HBcAg of an avian hepatitis virus (e.g., a hepatitis virus that infects stork or heron (e.g., SEQ. ID. NO. 20 and 22)), which has been codon-optimized for expression in humans and, which can be joined (e.g., in Cis) to a nucleic acid (preferably codon-optimized for expression in an animal or human) that encodes a heterologous antigen (e.g., a non-HBV antigen or a non-hepatitis antigen), such as, SEQ. ID. NOs. 2, 8, 10, 12, 14, 16, and 18. In some embodiments, it is preferred that the nucleic acid that encodes a heterologous protein or heterologous protein antigen is inserted in a spike region of the encoded HBcAg (e.g., between amino acid residues of about 87 to about 129). Compositions or mixtures that comprise, consist essentially of, or that consist of nucleic acids (e.g., in Trans) that encode an HBcAg of an avian hepatitis virus (e.g., a hepatitis virus that infects stork or heron), which has been codon-optimized for expression in humans (e.g., SEQ. ID. NO. 20 and 22) and a nucleic acid (preferably codon-optimized for expression in an animal or human) that encodes a heterologous peptide antigen (e.g., a non-HBV peptide or a non-hepatitis peptide), such as SEQ. ID. NOs. 2, 8, 10, 12, 14, 16, and 18 are also embodiments. Methods of using the aforementioned compositions to improve, enhance, or generate an immune response in a subject are also contemplated.

Some embodiments disclosed herein include an immunogenic composition comprising an isolated nucleic acid, which is codon optimized for expression in humans, encoding a hepatitis B virus core antigen (HBcAg) or a fragment thereof that is at least 50 amino acids in length; and an isolated nucleic acid, which is codon optimized for expression in humans, encoding a heterologous protein antigen. In some embodiments, the HBcAg is a human hepatitis antigen. The HBcAg may, in some embodiments, be a stork hepatitis antigen. In certain embodiments, the HBcAg is a heron hepatitis antigen.

Certain aspects of the immunogenic compositions disclosed herein comprise a full-length HBcAg. In some embodiments, the immunogenic composition comprises a fragment of a HBcAg that is at least 75 amino acids in length. In some other embodiments, the immunogenic composition comprises a fragment of a HBcAg that is at least 125 amino acids in length. Some embodiments have an immunogenic composition that comprises a fragment of a HBcAg that is at least 150 amino acids in length. In still another embodiment, the immunogenic composition comprises a fragment of a HBcAg that is at least 175 amino acids in length.

Certain embodiments disclosed herein include an immunogenic composition where the heterologous protein is a viral antigen, plant antigen, or animal antigen. In some embodiments, the heterologous protein is a viral antigen. Certain aspects of the immunogenic composition include a viral antigen that is a hepatitis antigen. In some embodiments, the hepatitis antigen is a hepatitis C virus (HCV) antigen. In some embodiments, the HCV antigen comprises NS3/4A. In other embodiments, the HCV antigen comprises NS5A. In still other embodiments, the HCV antigen comprises NS3/4A and NS5A. In some embodiments, the hepatitis antigen comprises a hepatitis B virus (HBV) antigen that is non-naturally occurring or in a non-naturally occurring position with respect to said HBcAg or fragment thereof. Some embodiments have an HBV antigen that comprises a human HBV surface antigen, a human HBV e antigen, a human HBcAg, a human HBV polymerase antigen, or a human HBV x antigen.

In certain aspects, said HBcAg or fragment thereof is a stork or heron hepatitis antigen and said HBV antigen is a human HBcAg. In some aspects, said HBcAg is a stork or heron hepatitis antigen and said HBV antigen is a human HBV e antigen.

In some embodiments, the heterologous protein is a plant antigen. In certain embodiments, the plant antigen comprises a birch antigen. In some embodiments, the heterologous protein is an animal antigen. In some aspects, the animal antigen comprises an ovalbumin antigen.

In some embodiments, the immunogenic compositions disclosed herein includes said isolated nucleic acid, which is codon optimized for expression in humans, encodes a full-length HBcAg and said isolated nucleic acid, which is codon optimized for expression in humans, encodes a heterologous protein and both isolated nucleic acids are in the same nucleic acid construct.

Another embodiment includes the immunogenic compositions disclosed herein includes said isolated nucleic acid, which is codon optimized for expression in humans, encodes a HBcAg and said isolated nucleic acid, which is codon optimized for expression in humans, encodes a heterologous protein and said nucleic acids are in separate nucleic acid constructs.

In some embodiments of the immunogenic composition, said HBcAg is a human hepatitis antigen and said heterologous protein comprises a HCV NS3/4A. In certain aspects,

said immunogenic composition comprises a nucleic acid of sequence SEQ. ID. NO. 10, SEQ. ID. NO. 2, or SEQ. ID. NO. 75.

In some embodiments of the immunogenic composition, said HBcAg is a stork hepatitis antigen and said heterologous protein comprises a HCV NS3/4A. In certain aspects, said immunogenic composition comprises a nucleic acid of sequence SEQ. ID. NO. 24, SEQ. ID. NO. 2, or SEQ. ID. NO. 24.

In some embodiments of the immunogenic composition, said HBcAg is a heron hepatitis antigen and said heterologous protein comprises a HCV NS3/4A. In certain aspects, said immunogenic composition comprises a nucleic acid of sequence SEQ. ID. NO. 22, SEQ. ID. NO. 2, or SEQ. ID. NO. 32.

In some embodiments of the immunogenic composition, said HBcAg is a human hepatitis antigen and said heterologous protein comprises a HCV NS5A. In certain aspects, said immunogenic composition comprises a nucleic acid of sequence SEQ. ID. NO. 10 or SEQ. ID. NO. 8.

In some embodiments of the immunogenic composition, said HBcAg is a stork hepatitis antigen and said heterologous protein comprises a HCV NS5A. In certain aspects, said immunogenic composition comprises a nucleic acid of sequence SEQ. ID. NO. 20, SEQ. ID. NO. 2, or SEQ. ID. NO. 28.

In some embodiments of the immunogenic composition, said HBcAg is a heron hepatitis antigen and said heterologous protein comprises a HCV NS5A. In certain aspects, said immunogenic composition comprises a nucleic acid of sequence SEQ. ID. NO. 22, SEQ. ID. NO. 8, or SEQ. ID. NO. 42.

In some embodiments of the immunogenic composition, HBcAg is a human hepatitis antigen and said heterologous protein comprises a HBV e antigen. In certain aspects, said immunogenic composition comprises a nucleic acid of sequence SEQ. ID. NO. 10, SEQ. ID. NO. 12, or SEQ. ID. No. 14.

In some embodiments of the immunogenic composition, said HBcAg is a stork hepatitis antigen and said heterologous protein comprises a HBV e antigen. In certain aspects, said immunogenic composition comprises a nucleic acid of sequence SEQ. ID. NO. 20, SEQ. ID. NO. 12, SEQ. ID. No. 14, SEQ. ID. NO. 44 or SEQ. ID. NO. 46.

In some embodiments of the immunogenic composition, said HBcAg is a heron hepatitis antigen and said heterologous protein comprises a HBV e antigen. In certain aspects, said immunogenic composition comprises a nucleic acid of sequence SEQ. ID. NO. 22, SEQ. ID. NO. 12, SEQ. ID. No. 14, SEQ. ID. NO. 48 or SEQ. ID. NO. 50.

In some embodiments of the immunogenic composition, said HBcAg is a stork hepatitis antigen and said heterologous protein comprises a HBcAg that is a human hepatitis antigen. In certain aspects, said immunogenic composition comprises a nucleic acid of sequence SEQ. ID. NO. 20, SEQ. ID. NO. 10, or SEQ. ID. NO. 52.

In some embodiments of the immunogenic composition, said HBcAg is a heron hepatitis antigen and said heterologous protein comprises a HBcAg that is a human hepatitis antigen. In certain aspects, said immunogenic composition comprises a nucleic acid of sequence SEQ. ID. NO. 22, SEQ. ID. NO. 10, or SEQ. ID. NO. 54.

In some embodiments of the immunogenic composition, said HBcAg is a human hepatitis antigen and said heterologous protein comprises a birch antigen. In certain aspects, said immunogenic composition comprises a nucleic acid of sequence SEQ. ID. NO. 10 or SEQ. ID. NO. 18.

In some embodiments of the immunogenic composition, said HBcAg is a stork hepatitis antigen and said heterologous protein comprises a birch antigen. In certain aspects, said immunogenic composition comprises a nucleic acid of sequence SEQ. ID. NO. 20, SEQ. ID. NO. 18, or SEQ. ID. NO. 56.

In some embodiments of the immunogenic composition, said HBcAg is a heron hepatitis antigen and said heterologous protein comprises a birch antigen. In certain aspects, said immunogenic composition comprises a nucleic acid of sequence SEQ. ID. NO. 22, SEQ. ID. NO. 18, or SEQ. ID. NO. 58.

In some embodiments of the immunogenic composition, said HBcAg is a human hepatitis antigen and said heterologous protein comprises an ovalbumin antigen. In certain aspects, said immunogenic composition comprises a nucleic acid of sequence SEQ. ID. NO. 10, or SEQ. ID. NO. 16.

In some embodiments of the immunogenic composition, said HBcAg is a stork hepatitis antigen and said heterologous protein comprises an ovalbumin antigen. In certain aspects, said immunogenic composition comprises a nucleic acid of sequence SEQ. ID. NO. 22, SEQ. ID. NO. 16, or SEQ. ID. NO. 60.

In some embodiments of the immunogenic composition, said HBcAg is a heron hepatitis antigen and said heterologous protein comprises an ovalbumin antigen. In certain aspects, said immunogenic composition comprises a nucleic acid of sequence SEQ. ID. NO. 22, SEQ. ID. NO. 16, or SEQ. ID. NO. 62.

In some embodiments of the immunogenic composition, said HBcAg is a stork or heron hepatitis antigen and said heterologous protein comprises HCV NS3/4A and NS5A, and an NS3 protease cleavage site between NS5A and said HBcAg.

Some embodiments of the immunogenic composition disclosed herein comprise an isolated HBcAg that is a stork or heron hepatitis antigen or a fragment thereof that is at least 50 amino acids in length and a heterologous protein, wherein said heterologous protein is in admixture with said HBcAg and not bound thereto. In some embodiments, the HBcAg is a stork hepatitis antigen. In some embodiments, the HBcAg is a heron hepatitis antigen.

Certain aspects of the immunogenic composition comprises a full-length HBcAg. Other aspects of the immunogenic composition comprises a fragment of a HBcAg that is at least 75 amino acids in length. In some embodiments, the immunogenic composition comprises a fragment of a HBcAg that is at least 125 amino acids in length. In some embodiments, the immunogenic composition comprises a fragment of a HBcAg that is at least 150 amino acids in length. In some embodiments, the immunogenic composition comprises a fragment of a HBcAg that is at least 175 amino acids in length.

In some embodiments, the heterologous protein is a viral antigen, plant antigen, or animal antigen. In some embodiments, the heterologous protein is a viral antigen. In some embodiments, the viral antigen is a hepatitis antigen. In certain embodiments, the hepatitis antigen is a hepatitis C virus (HCV) antigen. In still other embodiments, the HCV antigen comprises NS3/4A. In another embodiment, the HCV antigen comprises NS5A. In some embodiments, the HCV antigen comprises NS3/4A and NS5A. In some embodiments, the hepatitis antigen comprises a hepatitis B virus (HBV) antigen that is non-naturally occurring or in a non-naturally occurring position with respect to said HBcAg or fragment thereof.

In some aspects, the heterologous protein is a plant antigen. In some embodiments, the plant antigen comprises a birch antigen.

In some embodiments, the heterologous protein is an animal antigen. In an embodiment, the animal antigen comprises an ovalbumin antigen.

Some embodiments of the immunogenic composition disclosed herein, where the HBV antigen comprises a human HBV surface antigen, a human HBV e antigen, a human HBcAg, a human HBV polymerase antigen, or a human HBV x antigen. In some embodiments, said HBcAg or fragment thereof is a stork or heron hepatitis antigen and said HBV antigen is a human HBcAg. In some embodiments, said HBcAg is a stork or heron hepatitis antigen and said HBV antigen is a human HBV e antigen.

Some embodiments of the immunogenic composition of disclosed herein further comprise an isolated nucleic acid encoding a protein selected from the group consisting of interleukin (IL) 2, IL12, IL15, IL21, IL28b, galactose transferase (gal transferase), and a toll-like receptor ligand (TLR) or an adjuvant selected from the group consisting of IL2, IL12, IL15, IL21, IL28b, gal transferase, a TLR, ribavirin, alum, CpGs, and an oil.

Some embodiments include an isolated nucleic acid encoding an HBcAg fusion protein comprising an isolated nucleic acid, which is codon optimized for expression in humans, encoding a hepatitis B virus core antigen (HBcAg) or a fragment thereof that is at least 50 amino acids in length joined to an isolated nucleic acid, which is codon optimized for expression in humans, encoding a heterologous protein.

In some embodiments, the HBcAg is a human hepatitis antigen. In still another embodiment, the HBcAg is a stork hepatitis antigen. In some embodiments, the HBcAg is a heron hepatitis antigen.

In some embodiments, the nucleic acid comprises a full-length HBcAg. In other embodiments, the nucleic acid comprises a fragment of a HBcAg that is at least 75 amino acids in length. In some embodiments, the nucleic acid comprises a fragment of a HBcAg that is at least 125 amino acids in length. In some other embodiments, the nucleic acid comprises a fragment of a HBcAg that is at least 150 amino acids in length. In another embodiment, the nucleic acid comprises a fragment of a HBcAg that is at least 175 amino acids in length.

In certain aspects of the isolated nucleic acid disclosed herein, the heterologous protein is a viral antigen, plant antigen, or animal antigen. In some embodiments, the heterologous protein is a viral antigen. In some embodiments, the viral antigen is a hepatitis antigen. In certain embodiments, the hepatitis antigen is a hepatitis C virus (HCV) antigen. In some embodiments, the HCV antigen comprises NS3/4A. In some embodiments, the HCV antigen comprises NS5A. In some other embodiments, the HCV antigen comprises NS3/4A and NS5A. In an embodiment, the hepatitis antigen comprises a hepatitis B virus (HBV) antigen that is non-naturally occurring or in a non-naturally occurring position with respect to said HBcAg or fragment thereof. In some embodiments, the HBV antigen comprises a human HBV surface antigen, a human HBV e antigen, a human HBcAg, a human HBV polymerase antigen, or a human HBV x antigen.

In some embodiments, heterologous protein is a plant antigen. In an embodiment, the plant antigen comprises a birch antigen.

In some embodiments, the heterologous protein is an animal antigen. In certain aspects, the animal antigen comprises an ovalbumin antigen.

In some embodiments, said HBcAg or fragment thereof is a stork or heron hepatitis antigen and said HBV antigen is a human HBcAg. In some embodiments, said HBcAg is a stork or heron hepatitis antigen and said HBV antigen is a human HBV e antigen.

Some embodiments of isolated nucleic acid include nucleic acid is selected from the group consisting of SEQ. ID. Nos. 24, 26, 28, 30, 32, 34, 36, 38, 40, 42, 44, 46, 48, 50, 51, 52, 54, 56, 58, 60, 62, 64, 66, 68, 73, 75, 77, 79, 81, 83, 85, 87, 89, 103 and 105.

Some embodiments disclosed herein are proteins encoded by the isolated nucleic acid of disclosed herein.

Certain aspects of the present invention include the use of a nucleic acid, protein, or immunogenic composition disclosed herein to prepare a medicament for generating an immune response in a subject to said heterologous protein.

Some embodiments disclosed herein are a method of using one or more of the compositions disclosed herein to produce an immune response in a subject comprising providing one or more of the compositions disclosed herein; and administering said composition to said subject.

Some embodiments disclosed herein are a method of improving an immune response to a heterologous protein in a subject comprising providing one or more of the compositions disclosed herein; administering said composition to said subject; and measuring an immune response to said heterologous protein.

In some embodiments, the methods disclosed herein have said composition is administered by injection. In some embodiments, said injection is intra muscular, dermal, or subdermal. In some embodiments, the method further comprises providing an electrical stimulation. In certain aspects, said electrical stimulation is electroporation.

In some embodiments, the methods include said isolated nucleic acid that encodes a full-length HBcAg is provided and the isolated nucleic acid that encodes a full-length HBcAg is administered separately from the isolated nucleic acid that encodes a heterologous protein. In some embodiments, the methods include said isolated nucleic acid that encodes a full-length HBcAg is administered before said isolated nucleic acid that encodes a heterologous protein.

Some embodiments of the methods disclosed herein, wherein an isolated nucleic acid that encodes a full-length HBcAg is provided and the isolated nucleic acid that encodes a full-length HBcAg is administered in admixture with the isolated nucleic acid that encodes a heterologous protein.

Some embodiments include an immunogenic composition comprising a nucleic acid, which is codon-optimized for expression in humans, encoding a hepatitis B virus core antigen (HBcAg) and a heterologous protein antigen.

Some embodiments include an immunogenic composition comprising a hepatitis B virus core antigen (HBcAg) protein and a nucleic acid, which is codon-optimized for expression in humans, encoding heterologous protein antigen.

In some embodiments, said nucleic acid encoding HBcAg or said HBcAg protein is derived from stork or heron hepatitis virus.

Some embodiments include a method of promoting an immune response in a subject comprising coadministering a nucleic acid, which is codon-optimized for expression in humans, encoding a hepatitis B virus core antigen (HBcAg) and a heterologous protein antigen.

Some embodiments include a method of promoting an immune response in a subject comprising: coadministering a hepatitis B virus core antigen (HBcAg) protein and a nucleic acid, which is codon-optimized for expression in humans, encoding a heterologous protein antigen.

### Isolated Nucleic Acids and Proteins

Disclosed herein are compositions that comprise isolated nucleic acids encoding HBcAg, or a fragment thereof, joined to (e.g., flanking or juxtaposed to) an isolated nucleic acid encoding a heterologous protein. Accordingly, the isolated nucleic acid may, in some embodiments, encode a fusion protein that includes at least a fragment of HBcAg, and a heterologous protein. Polypeptides encoded by said isolated nucleic acids are also embodiments of the present invention.

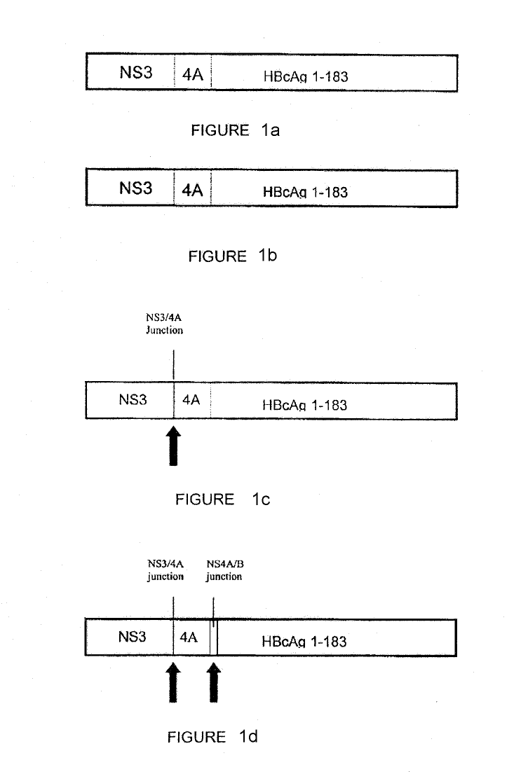

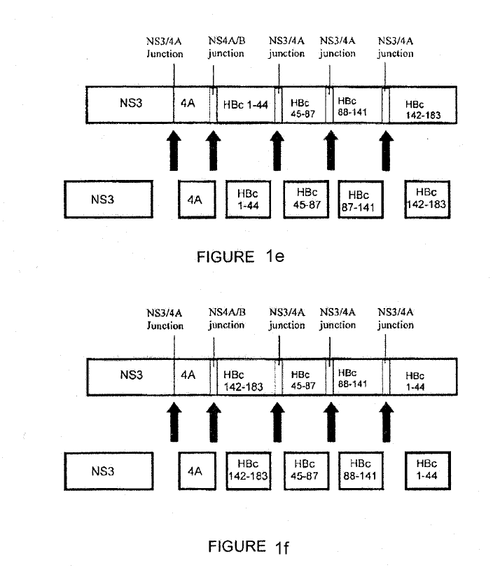

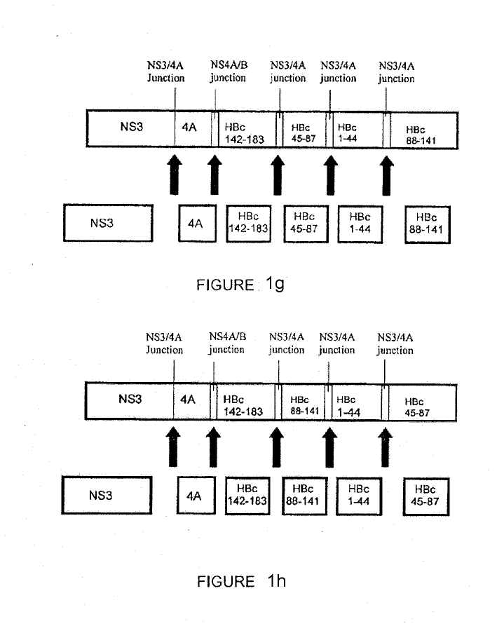

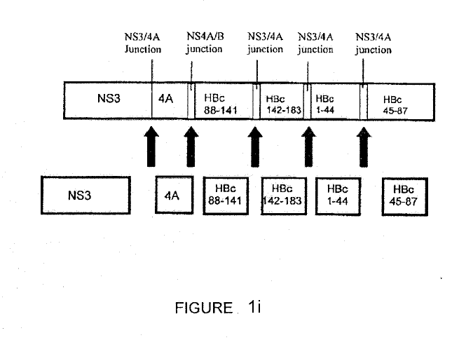

FIG. 1(a-i) shows various embodiments of constructs that include HBcAg joined to HCV NS3/4A, which is an exemplary heterologous protein (and an antigen) within the scope of the present invention. FIG. 1a shows an exemplary construct having HCV NS3/4A joined to HBcAg, which is exemplified by SEQ. ID. No. 22. The sequence includes portions that encode HCV NS3/4A juxtaposed to HBcAg, and therefore encode a fusion protein (e.g., SEQ. ID. No. 23 encoded in SEQ. ID. No. 24). Similarly, FIG. 1b shows another construct having HCV NS3/4A joined to HBcAg, which encodes a mutant NS3 polypeptide and is exemplified by SEQ. ID. No. 26

FIGS. 1(c-i) show various embodiments of constructs that include HBcAg joined to HCV NS3/4A, where one or more cleavage sites are encoded between portions of the polypeptides encoded thereon. FIG. 1(c) encodes an NS3/4A junction between the NS3 and NS4A, and therefore encodes a protein configured to be cleaved by NS3 protease to provide an NS3 polypeptide, and an NS4A-HBcAg fusion protein. SEQ. ID. No. 38 is an exemplary sequence encoding the protein in SEQ. ID. No. 37 and includes the same features as the construct shown in FIG. 1(c). Furthermore, FIG. 1(d) shows a construct having two cleavage sites, where the construct encodes a protein that may be cleaved to form NS3, NS4A and HBcAg polypeptides. SEQ. ID. No. 64 exemplifies a nucleic acid sequence sharing the same features shown in FIG. 1(d). Finally, FIGS. 1(e-i) show embodiments of constructs 5 cleavage sites positioned between various portions of the encoded polypeptide. These constructs include 3 cleavage sites between fragments of HBcAg, and therefore encode a polypeptide configured to be cleaved by NS3 protease to form at least 4 fragments of HBcAg. Non-limiting examples of the constructs disclosed in FIGS. 1(e-i) are SEQ. ID. Nos. 81, 83, 85, 87 and 89, respectively.

The nucleocapsid or core antigen HBcAg of HBV is an immunogenic particle composed of 180 subunits of a single protein chain. HBcAg has been disclosed as an immunogenic moiety that stimulates the T cell response of an immunized host animal. See, e.g, U.S. Pat. No. 4,818,527, U.S. Pat. No. 4,882,145 and U.S. Pat. No. 5,143,726, each of which is hereby incorporated by reference in their entirety. It can be used as a carrier for several peptidic epitopes covalently linked by genetic engineering as well as for chemically coupled protein antigens. (See Sallberg et al. (1998) Human Gene Therapy 9:1719-29). In addition, HBcAg is non-cytotoxic in humans. Accordingly, it was contemplated that HBcAg is useful in genetic constructs for generating or enhancing an immune response to an accompanied target antigen (e.g., in constructs that encode a TCE derived from a pathogen).

Current listings of exemplary HBcAg sequences are publicly available at the National Center for Biotechnology Information (NCBI) world-wide web site. HBcAg nucleic acid sequences (including novel HBcAg regions) can also be isolated from subjects (e.g., humans) infected with HBV. DNA obtained from a patient infected with HBV can be amplified using PCR or another amplification technique.

For a review of PCR technology, see Molecular Cloning to Genetic Engineering White, B. A. Ed. in Methods in Molecular Biology 67: Humana Press, Totowa (1997) and the publication entitled “PCR Methods and Applications” (1991, Cold Spring Harbor Laboratory Press). For amplification of mRNAs, it is within the scope of the invention to reverse transcribe mRNA into cDNA followed by PCR (RT-PCR); or, to use a single enzyme for both steps as described in U.S. Pat. No. 5,322,770. Another technique involves the use of Reverse Transcriptase Asymmetric Gap Ligase Chain Reaction (RT-AGLCR), as described by Marshall R. L. et al. (PCR Methods and Applications 4:80-84, 1994).

The source of the HBcAg sequences that are included in the isolated nucleic acids described herein is not particularly limited. Accordingly, embodiments described herein may utilize an isolated nucleic acid that encodes an HBcAg derived from a hepatitis virus capable of infecting animals of any species, including but limited to, humans, non-human primates (e.g., baboons, monkeys, and chimpanzees), rodents, mice, reptiles, birds (e.g., stork and heron), pigs, micro-pigs, goats, dogs and cats. In some embodiments, the HBcAg is selected from a human hepatitis antigen or an avian hepatitis antigen. Particularly preferred are the stork hepatitis antigen and a heron hepatitis antigen.

In certain embodiments, the HBcAg sequences described herein have variations in nucleotide and/or amino acid sequences, compared to native HBcAg sequences and are referred to as HBcAg variants or mutants. As used herein, the term “native” refers to naturally occurring HBV sequences (e.g., available HBV isotypes). Variants may include a substitution, deletion, mutation or insertion of one or more nucleotides, amino acids, or codons encoding the HBcAg sequence, which may result in a change in the amino acid sequence of the HBcAg polypeptide, as compared with the native sequence. Variants or mutants can be engineered, for example, using any of the techniques and guidelines for conservative and non-conservative mutations set forth, for instance, in U.S. Pat. No. 5,364,934, which is hereby incorporated by reference in its entirety.

Accordingly, when the term “consisting essentially of” is used, in some contexts, variants or mutants of an HBcAg sequence or of a particular antigen sequence are intended to be encompassed. That is, in some contexts and in some embodiments, the variants or mutants of the sequences disclosed herein (e.g., SEQ. ID. No. 10) are equivalents because the variation or mutation in sequence does not change or materially affect the basic and novel characteristics of the claimed invention.

A codon-optimized HBcAg can, in some embodiments, be encoded within the isolated nucleic acid. A codon-optimized sequence may, in some embodiments, be obtained by substituting codons in an existing sequence with codons more frequently used in the intended host subject (e.g., a human). Some examples include, but are not limited to, codon-optimized nucleic acids encoding human HBcAg (e.g., SEQ. ID. No. 10), codon-optimized nucleic acids encoding stork HBcAg (e.g., SEQ. ID. No. 20), and codon-optimized nucleic acids encoding heron HBcAg (e.g., SEQ. ID. No. 22).

The isolated nucleic acids can encode the full-length HBcAg in certain embodiments (e.g., SEQ. ID. No. 71). However, fragments of the HBcAg may also be encoded with the nucleic acid in certain embodiments. A fragment of the HBcAg sequence can comprise at least, equal to, greater than, or less than, or any number in between 3, 5, 10, 20, 50, 75, 100, 125, 150, or 175 consecutive amino acids of a natural or synthetic HBcAg polypeptide (e.g., a naturally occurring isotype or a codon-optimized or otherwise modified HBcAg polypeptide). FIGS. 1(e-i) illustrate several constructs encoding fragments of HBcAg that are between about 40 to about 60 amino acids in length.

Some embodiments include, for example, one or more of the HBcAg nucleic acid or protein sequences disclosed in International Patent Application Publication Number WO 20091130588, which designated the United States and was published in English, the disclosure of which is hereby expressly incorporated by reference in its entirety.

Meanwhile, the isolated nucleic acid encoding HBcAg may also be joined to an isolated nucleic acid encoding a heterologous protein. The heterologous protein may generally vary in the same manner discussed above with respect to the HBcAg. Thus, in some embodiments, the isolated nucleic acid sequences may encode native, variants or mutants of a heterologous protein, and these nucleic acids may also be codon-optimized (e.g., a codon-optimized nucleic acid encoding HCV NS3/4A from the human hepatitis virus in SEQ. ID. No. 2, a codon-optimized nucleic acid encoding NS5A from the human hepatitis virus in SEQ. ID. No. 8, codon-optimized nucleic acid encoding HBV HBcAg from the human hepatitis virus in SEQ. ID. No. 10, codon-optimized nucleic acid encoding HBV HBcAg from the human hepatitis virus in SEQ. ID. Nos. 12 and 14, codon-optimized nucleic acid encoding ovalbumin in SEQ. ID. No. 16, codon-optimized nucleic acid encoding birch antigen in SEQ. ID. No. 18). In some embodiments, the isolated nucleic acid encodes a fragment of the heterologous protein. In some embodiments, all of the vaccine sequences include a Kozak sequence (e.g., SEQ. ID. No. 106).

The heterologous protein, in some embodiments, can be an antigen, such as a plant antigen (e.g., birch antigen), viral antigen, or an animal antigen (e.g., ovalbumin antigen). The antigen may also be a hepatitis antigen, for example a hepatitis B virus (HBV) antigen or a hepatitis C virus (HCV) antigen. The HCV antigens can be from viruses known to infect animals of any species, including, but not limited to, amphibians, reptiles, birds—such as stork, and heron, mice, hamsters, rats, rabbits, guinea pigs, woodchucks, pigs, micro-pigs, goats, dogs, cats, humans and non-human primates (e.g., baboons, monkeys, and chimpanzees). Similarly, the HBV antigens can be from viruses known to infect animals of any species, including, but not limited to, amphibians, reptiles, birds—such as stork, and heron, mice, rodents, pigs, micro-pigs, goats, dogs, cats, humans and non-human primates (e.g., baboons, monkeys, and chimpanzees). In certain embodiments, the antigen is a HCV antigen selected from NS3/4A, NS5A, and combinations thereof. In certain embodiments, the antigen is a HBV antigen selected from a HBV surface antigen, HBV e antigen, human HBcAg, a human HBV polymerase antigen, a human HBV x antigen, and combinations thereof.

If the isolated nucleotide encodes a heterologous protein that is an HBV antigen, the heterologous protein can be substantially different than the HBcAg also encoded in the isolated nucleotide. As an example, the isolated nucleic acid may include a nucleic acid encoding HBcAg, which is joined to an isolated nucleic acid encoding a heterologous protein, where the heterologous protein is an HBV antigen that is non-naturally occurring or in a non-naturally occurring position with respect to the HBcAg. SEQ. ID. No. 54 is an exemplary nucleic acid that includes this feature because it encodes heron HBcAg joined to human HBcAg. Without being limited to any particular designation, the human HBcAg is an HBV antigen that is in a non-naturally occurring position with respect to the heron HBcAg. Conversely, the heron HBcAg may be designated as the HBV antigen that is in a non-naturally occurring position with respect to the human HBcAg.

Some embodiments have an isolated nucleic acid that encodes at least a stork or heron HBcAg antigen, or a fragment thereof, and human HBcAg, or a fragment thereof (e.g., SEQ. ID. No. 52 and 54). In certain embodiments, the isolated nucleic acid encodes at least stork or heron HBcAg antigen, or a fragment thereof, and human HBV e antigen, or a fragment thereof (e.g., SEQ. ID. 44 and 46).

Some embodiments include, for example, one or more heterologous proteins, or isolated nucleic acids encoding the same, in International Patent Application Publication Number WO 20091130588, which designated the United States and was published in English, the disclosure of which is hereby expressly incorporated by reference in its entirety. As an example, various HCV HS3/4A polypeptides, and fragments of HCV HS3/4A polypeptides, are disclosed within WO 20091130588 which may be included in the isolated nucleic acids.

Non-limiting examples of isolated nucleic acids encoding HBcAg, or a fragment thereof, joined to an isolated nucleic acid encoding a heterologous protein, include, but are not limited to: (1) stork HBcAg joined to HCV NS3/4A (e.g., SEQ. ID. No. 24 and 26); (2) heron HBcAg joined to HCV NS3/4A (e.g., SEQ. ID. No. 36); (3) stork HBcAg joined to HCV NS5A (e.g., SEQ. ID. No. 40); (4) heron HBcAg joined to HCV NS5A (e.g., SEQ. ID. No. 42); (5) stork HBcAg joined to human HBV e antigen (e.g., SEQ. ID. No. 44 and 46); (6) heron HBcAg joined to human HBV e antigen (e.g., SEQ. ID. No. 48 and 50); (7) stork HBcAg and human HBcAg (e.g., SEQ. ID. No. 52 and 103); (8) heron HBcAg joined to human HBcAg (e.g., SEQ. ID. No. 50 and 105); (9) stork HBcAg joined to birch antigen (e.g., SEQ. ID. No. 56); (10) heron HBcAg joined to birch antigen (e.g., SEQ. ID. No. 58); (11) stork HBcAg joined to ovalbumin antigen (e.g., SEQ. ID. No. 60); and (12) stork HBcAg joined to ovalbumin antigen (e.g., SEQ. ID. No. 62).

In some aspects, as discussed above, the isolated nucleic acid includes one or more NS3 protease cleavage sites, wherein the NS3 protease cleavage site is at a non-naturally occurring position. Examples of cleavage sites that may be included in the isolated nucleic acid include, but are not limited to, SEQ. ID. No. 4 and 6. In certain embodiments, the NS3 protease cleavage site is between the sequences encoding HBcAg and the heterologous protein. Thus, in some embodiments, the isolated nucleic acids encode a fusion protein, which may be cleaved by NS3 protease. In other aspects, the isolated nucleic acid encodes two or more fragments of HBcAg having a cleavage site between the two encoded fragments. Accordingly, the isolated nucleic acid encoding fragments of HBcAg, and therefore encodes a protein that is configured to be cleaved by NS3 protease to form HBcAg fragments.

Some embodiments of the isolated nucleic acid include an isolated nucleic acid encoding HBcAg, or a fragment thereof, joined to an isolated nucleic acid encoding heterologous protein, wherein the heterologous protein is HCV NS3/4A (e.g., SEQ. ID. No. 24). In certain embodiments, the isolated nucleic acid encodes an NS3 protease cleavage site between the isolated nucleic acid encoding HCV NS3/4A and the isolated nucleic acid encoding HBcAg (e.g., SEQ. ID. No. 30).

Embodiments of the isolated nucleic acid include HBcAg and a plurality of isolated nucleic acids encoding antigens, each of the isolated nucleic acids being joined together and having an HCV protease cleavage site in between. As an example, SEQ. ID. Nos. 64, 66, and 68 include NS3/4A antigen, NS5A antigen, and HBcAg antigen having an HCV protease cleavage site between each antigen.

Some embodiments of the isolated nucleic acids disclosed herein encode a fragment of human HBcAg between (i.e., joined at both ends to) fragments of avian HBcAg (e.g., stork or heron HBcAg). Thus, for example, the isolated nucleic acid may encode a polypeptide, where the polypeptide comprises, consists essentially of, or consists of avian HBcAg having a fragment of human HBcAg inserted into said avian HBcAg. In some aspects, the human HBcAg fragment is inserted into at least a portion, or all, of the spike region of the avian HBcAg (i.e., the region of HBcAg displayed on the surface the HBcAg capsid). Preferably, the human HBcAg is encoded into any, or all, of the amino acid positions 87 to 129 in the nucleic acid encoding avian HBcAg (e.g., codon-optimized stork HBcAg (e.g., SEQ. ID. No. 20) or codon-optimized heron HBcAg (e.g., SEQ. ID. No. 22)). In a preferred embodiment, the isolated nucleic acid encodes about a 43 amino acid fragment of human HBcAg inserted into amino acid positions of about 87 to about 129 of an avian HBcAg (e.g., an isolated nucleic acid that encodes codon-optimized stork HBcAg having 43 amino acid fragment of human HBcAg inserted at amino acid positions 87 to 129 (e.g., SEQ. ID. No. 103, which encodes the fusion protein in SEQ. ID. No. 102), or an isolated nucleic acid that encodes codon-optimized heron HBcAg having a 43 amino acid fragment of human HBcAg is inserted at amino acid positions 87 to 129 (e.g., SEQ. ID. No. 105, which encodes the fusion protein in SEQ. ID. No. 104)).

As would be appreciated by a person of ordinary skill, the proteins encoded in the isolated nucleic acids disclosed herein may be obtained using known methods. As an example, the nucleic acids may be inserted into an appropriate plasmid, which is subsequently inserted into to cells that express the protein. Other methods for obtaining the encoded proteins are also known. Accordingly, the scope of the present application includes the proteins that can be obtained from the isolated nucleic acids disclosed herein. For example, SEQ. ID. 23 describes a protein that can be obtained from the expression of SEQ. ID. 24. Thus, embodiments of the present invention also include, but are not limited to, proteins having the sequences in SEQ. ID. Nos. 23, 25, 27, 29, 31, 33, 35, 37, 39, 41, 43, 45, 47, 49, 51, 53, 55, 57, 59, 61, 63, 65, 67, 69, 70, 72, 74, 76, 78, 80, 82, 84, 86, 88, 102 and 104.

### Immunogenic Compositions Comprising Nucleic Acids

Disclosed herein are immunogenic compositions relating to genetic constructs that include nucleic acids encoding HBcAg, or a fragment thereof, and nucleic acids encoding a heterologous protein. In some embodiments, both sequences are in the same nucleic acid construct (e.g., the same plasmid). In certain embodiments, both sequences are in separate nucleic acid constructs. Some embodiments of the immunogenic compositions described herein include any of the isolated nucleic acids discussed above, wherein a nucleic acid encoding HBcAg, or a fragment thereof, is joined to a nucleic acid encoding a heterologous protein. Some embodiments of the immunogenic compositions disclosed herein include one or proteins encoded by a nucleic acid described herein.

The source of the HBcAg that is encoded in the nucleic acid is not particularly limited. Accordingly, the nucleic acid contemplated for the immunogenic compositions described herein can be nucleic acids from viruses known to infect animals of any species, including but limited to, humans, mice, reptiles, birds (e.g., stork and heron), rodents, pigs, micro-pigs, goats, dogs, cats, and non-human primates (e.g., baboons, monkeys, and chimpanzees), as mentioned above. In some embodiments, the HBcAg is selected from a human hepatitis antigen, an avian hepatitis antigen, a stork hepatitis antigen, and a heron hepatitis antigen.

The sequences encoding HBcAg can generally be the same as those discussed above with respect to the isolated nucleic acids. Thus, in some embodiments, any of the nucleic acid sequences described above that include HBcAg may be used in the immunogenic composition. As an example, the isolated nucleic acid may include native (e.g., SEQ. ID. No. 71) or variant HBcAg or mutant HBcAg, and the nucleic acid may also be codon-optimized (e.g., SEQ. ID. No. 22). In some embodiments, the isolated nucleic acid encodes a fragment of HBcAg, as described above with respect to the isolated nucleic acids. For example, fragment of the HBcAg sequence can comprise at least, equal to, greater than, or less than, or any number in between 3, 5, 10, 20, 50, 75, 100, 125, 150, or 175 consecutive amino acids of a natural or synthetic HBcAg polypeptide. A full-length HBcAg can also be encoded in an isolated nucleic acid included within the immunogenic composition.

Some embodiments include nucleic acids that have homology or sequence identity to any one of the nucleic acid sequences disclosed herein (e.g. SEQ. ID. Nos. 24, 26, 28, 30, 32, 34, 36, 38, 40, 42, 44, 46, 48, 50, 52, 54, 56, 58, 60, 62, 64, 66, 68, 70, 102, 104 etc.). In some embodiments, said homologous nucleic acids generate, enhance, or improve an immune response, as defined above. Several techniques exist to determine nucleic acid or protein sequence homology. Thus, embodiments of the nucleic acids can have from 70% homology or sequence identity to 100% homology or sequence identity to any one of the nucleic acid sequences or protein sequences disclosed herein. That is, embodiments can have at least, equal to or any number between about 70.0%, 71.0%, 72.0%, 73.0%, 74.0%, 75.0%, 76.0%, 77.0%, 78.0%, 79.0%, 80.0%, 81.0%, 82.0%, 83.0%, 84.0%, 85.0%, 86.0%, 87.0%, 88.0%, 89.0%, 90.0%, 91.0%, 92.0%, 93.0%, 94.0%, 95.0%, 96.0%, 97.0%, 98.0%, 99.0%, and 100.0% homology or sequence identity to any one of the polypeptide or nucleic acid sequences disclosed herein.

Several homology or sequence identity searching programs based on nucleic acid sequences are known in the art and can be used to identify molecules that are homologous. In one approach, a percent sequence identity can be determined by standard methods that are commonly used to compare the similarity and position of the base pairs of two nucleic acids. Using a computer program such as BLAST or FASTA, two sequences can be aligned for optimal matching of their respective base pairs (either along the full length of one or both sequences, or along a predetermined portion of one or both sequences). Such programs provide “default” opening penalty and a “default” gap penalty, and a scoring matrix such as PAM 250 (a standard scoring matrix; see Dayhoff et al., in: Atlas of Protein Sequence and Structure, Vol. 5, Supp. 3 (1978)) can be used in conjunction with the computer program.

Some embodiments included isolated nucleic acids having sufficient homology or sequence identity to any one of the nucleic acid sequences disclosed herein such that hybridization will occur between the isolated nucleic acid and any one of the nucleic acids sequences disclosed herein. In some aspects, hybridization occurs under usual washing conditions in Southern hybridization, that is, at a salt concentration corresponding to 0.1 times saline sodium citrate (SSC) and 0.1% SDS at 37° C. (low stringency), preferably 0.1 times SSC and 0.1% SDS at 60° C. (medium stringency), and more preferably 0.1 times SSC and 0.1% SDS at 65° C. (high stringency). In certain aspects, the nucleic acid embodiments have a percentage of consecutive bases that hybridize under stringent conditions with any one of the nucleic acids sequences disclosed herein, where the number of consecutive bases is at least 40.0%, 41.0%, 42.0%, 43.0%, 44.0%, 45.0%, 46.0%, 47.0%, 48.0%, 49.0%, 50.0%, 51.0%, 52.0%, 53.0%, 54.0%, 55.0%, 56.0%, 57.0%, 58.0%, 59.0%, 60.0%, 61.0%, 62.0%, 63.0%, 64.0%, 65.0%, 66.0%, 67.0%, 68.0%, 69.0%, 70.0%, 71.0%, 72.0%, 73.0%, 74.0%, 75.0%, 76.0%, 77.0%, 78.0%, 79.0%, 80.0%, 81.0%, 82.0%, 83.0%, 84.0%, 85.0%, 86.0%, 87.0%, 88.0%, 89.0%, 90.0%, 91.0%, 92.0%, 93.0%, 94.0%, 95.0%, 96.0%, 97.0%, 98.0%, 99.0%, and 100.0% of the total number of bases in the nucleic acid sequence.

Some embodiments of the immunogenic composition include a nucleic acid encoding a heterologous protein. The heterologous protein encoded by the nucleic acid, in some embodiments, can be an antigen, such as a plant antigen (e.g., birch antigen), viral antigen, or an animal antigen (e.g., ovalbumin antigen). The antigen may also be a hepatitis antigen, for example a hepatitis B virus (HBV) antigen or a hepatitis C virus (HCV) antigen. The HCV antigens can be from viruses known to infect animals of any species, including, but not limited to, amphibians, reptiles, birds (e.g., stork and heron) mice, hamsters, rats, rabbits, guinea pigs, woodchucks, pigs, micro-pigs, goats, dogs, cats, humans and non-human primates (e.g., baboons, monkeys, and chimpanzees). Similarly, the HBV antigens can be from viruses known to infect animals of any species, including, but not limited to, amphibians, reptiles, birds (e.g., stork and heron), and heron, mice, hamsters, rodents, pigs, micro-pigs, goats, dogs, cats, humans and non-human primates (e.g., baboons, monkeys, and chimpanzees). In certain embodiments, the antigen is a HCV antigen selected from NS3/4A, NS5A, and combinations thereof. In certain embodiments, the antigen is a HBV antigen selected from a HBV surface antigen, HBV e antigen, human HBcAg, a human HBV polymerase antigen, a human HBV x antigen, and combinations thereof.

Non-limiting examples of nucleic acids encoding heterologous proteins that may be included within the immunogenic composition include HCV NS3/4A (e.g., SEQ. ID. 2), HCV NS5A (e.g., SEQ. ID. 8), HBcAg (e.g., SEQ. ID. 10), HBV e antigen (e.g., SEQ. ID. 12 and 14), and ovalbumin (e.g., SEQ. ID. 16).

If the immunogenic composition includes an encoded heterologous protein that is an HBV antigen, the heterologous protein or nucleic acid encoding the heterologous protein can be substantially different than the HBcAg present in the immunogenic composition. As example, the immunogenic composition may include a nucleic acid encoding HBcAg, and a nucleic acid encoding a heterologous protein, which is an HBV antigen that is non-naturally occurring or in a non-naturally occurring position with respect to the HBcAg. The immunogenic composition may include, for example, a mixture of SEQ. ID. No. 10 and 22, and therefore includes two nucleic acids encoding substantially different HBV antigens (i.e., human HBcAg and heron HBcAg).

Non-limiting examples of mixtures of nucleic acid sequences encoding HBcAg, or a fragment thereof, and nucleic acid sequences encoding a heterologous protein, that may be included in the immunogenic compositions, include, but are not limited to, nucleic acid sequences encoding: (1) stork HBcAg and HCV NS3/4A (e.g., SEQ. ID. Nos. 20 and 2); (2) heron HBcAg and HCV NS3/4A (e.g., SEQ. ID. Nos. 22 and 2); (3) stork HBcAg and HCV NS5A (e.g., SEQ. ID. Nos. 20 and 8); (4) heron HBcAg and HCV NS5A (e.g., SEQ. ID. Nos. 22 and 8); (5) stork HBcAg and human HBV e antigen (e.g., SEQ. ID. Nos. 20 and 12); (6) heron HBcAg and human HBV e antigen (e.g., SEQ. ID. Nos. 22 and 12); (7) stork HBcAg and human HBcAg (e.g., SEQ. ID. Nos. 20 and 10); (8) heron HBcAg and human HBcAg (e.g., SEQ. ID. Nos. 22 and 10); (9) stork HBcAg and birch antigen (e.g., SEQ. ID. Nos. 20 and 18); (10) heron HBcAg and birch antigen (e.g., SEQ. ID. Nos. 22 and 18); (11) stork HBcAg and ovalbumin antigen (e.g., SEQ. ID. Nos. 20 and 16); and (12) stork HBcAg and ovalbumin antigen (e.g., SEQ. ID. Nos. 22 and 16).

Some embodiments of the immunogenic composition include the isolated nucleic acids described above, wherein the nucleic acid encoding HBcAg, or a fragment thereof, is joined to nucleic acid sequences encoding a heterologous protein. Accordingly, further exemplary compositions may include a nucleic acid encoding: (1) stork HBcAg joined to HCV NS3/4A (e.g., SEQ. ID. No. 24 and 26); (2) heron HBcAg joined to HCV NS3/4A (e.g., SEQ. ID. No. 36); (3) stork HBcAg joined to HCV NS5A (e.g., SEQ. ID. No. 40); (4) heron HBcAg joined to HCV NS5A (e.g., SEQ. ID. No. 42); (5) stork HBcAg joined to human HBV e antigen (e.g., SEQ. ID. No. 44 and 46); (6) heron HBcAg joined to human HBV e antigen (e.g., SEQ. ID. No. 48 and 50); (7) stork HBcAg joined to human HBcAg (e.g., SEQ. ID. No. 52 and 103); (8) heron HBcAg joined to human HBcAg (e.g., SEQ. ID. No. 50 and 105); (9) stork HBcAg joined to birch antigen (e.g., SEQ. ID. No. 56); (10) heron HBcAg joined to birch antigen (e.g., SEQ. ID. No. 58); (11) stork HBcAg joined to ovalbumin antigen (e.g., SEQ. ID. No. 60); and (12) stork HBcAg joined to ovalbumin antigen (e.g., SEQ. ID. No. 62).

It is contemplated that various other compounds may be included in one or more of the compositions. Some embodiments of the composition may further include an additional adjuvant. Non-limiting example of adjuvants that can be included are: interleukin-2 (IL2), interleukin-12 (IL 12), interleukin-15 (IL15), interleukin-21 (IL21), interleukin-28b (IL28b), galactosyl transferase, a toll-like receptor (TLR), ribavirin, alum, CpGs, or an oil. In some embodiments, the composition includes an isolated nucleic acid, or constructs comprising said nucleic acids, encoding a protein that is an adjuvant, such as IL2, IL12, IL15, IL21, IL28b, galactose transferase, a TLR, and the like. In certain aspects, the isolated nucleic acid encoding the protein which is an adjuvant may be in the same construct encoding HBcAg and/or the heterologous protein. In other aspects, the isolated nucleic acid encoding the protein, which is an adjuvant may be in a different construct than the construct encoding HBcAg and/or the heterologous protein.

The compositions described herein may also contain other ingredients or compounds in addition to nucleic acids and/or polypeptides, including, but not limited to, various other peptides, adjuvants, binding agents, excipients such as stabilizers (to promote long term storage), emulsifiers, thickening agents, salts, preservatives, solvents, dispersion media, coatings, antibacterial and antifungal agents, isotonic and absorption delaying agents and the like. See e.g., U.S. application Ser. No. 09/929,955 and U.S. application Ser. No. 09/930,591. These compositions are suitable for treatment of animals, particularly mammals, either as a preventive measure to avoid a disease or condition or as a therapeutic to treat animals already afflicted with a disease or condition.

Many other ingredients may also be present in the compositions provided herein. For example, the adjuvant and antigen can be employed in admixture with conventional excipients (e.g., pharmaceutically acceptable organic or inorganic carrier substances suitable for parenteral, enteral (e.g., oral) or topical application that do not deleteriously react with the therapeutic ingredients (e.g., construct encoding HBcAg). Suitable pharmaceutically acceptable carriers include, but are not limited to, water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid monoglycerides and diglycerides, pentaerythritol fatty acid esters, hydroxy methylcellulose, polyvinyl pyrrolidone, etc. Many more suitable carriers are described in Remmington's Pharmaceutical Sciences, 15th Edition, Easton:Mack Publishing Company, pages 1405-1412 and 1461-1487 (1975) and The National Formulary XIV, 14th Edition, Washington, American Pharmaceutical Association (1975).

### Immunogenic Compositions Comprising Polypeptides

Some of the embodiments described herein concern compositions that comprise, consist essentially of, or consist of polypeptides encoded by any of the nucleic acids disclosed herein. In some embodiments, the composition includes an admixture of HBcAg, or a fragment thereof, and a heterologous protein. In certain aspects, the composition includes a protein having HBcAg joined to a heterologous protein.

The HBcAg polypeptides that may be included in the immunogenic compositions can be any HBcAg polypeptide that can be encoded in the nucleic acids within the immunogenic composition of nucleic acids discussed above, or those encoded in the isolated nucleic acids discussed above. Thus, in some embodiments, the HBcAg is derived from a codon-optimized nucleic acid (e.g., SEQ. ID. No. 21 is derived from SEQ. ID. No. 22). The HBcAg may also be a native or variant form of the protein. Also, the composition may include a fragment of HBcAg. A fragment of HBcAg can comprise at least, equal to, greater than, or less than, or any number in between 3, 5, 10, 20, 50, 75, 100, 125, 150, or 175 consecutive amino acids of a natural or synthetic HBcAg polypeptide (e.g., a naturally occurring isotype or a codon-optimized or otherwise modified HBcAg polypeptide).

Some embodiments include polypeptides that have homology or sequence identity to any one of the polypeptide sequences disclosed herein (e.g. SEQ. ID. Nos. 23, 25, 27, 29, 31, 33, 35, 37, 39, 41, 43, 45, 47, 49, 51, 53, 55, 57, 59, 61, 63, 65, 67, 69, 70, 102, 104, etc.). In some embodiments, said polypeptides generate, enhance, or improve an immune response, as defined above. Several techniques exist to determine protein sequence homology or sequence identity. Thus, embodiments of the polypeptides can have from 70% homology to 100% homology or sequence identity to any one of the polypeptides disclosed herein. That is, embodiments can have at least, equal to, or any number in between about 70.0%, 71.0%, 72.0%, 73.0%, 74.0%, 75.0%, 76.0%, 77.0%, 78.0%, 79.0%, 80.0%, 81.0%, 82.0%, 83.0%, 84.0%, 85.0%, 86.0%, 87.0%, 88.0%, 89.0%, 90.0%, 91.0%, 92.0%, 93.0%, 94.0%, 95.0%, 96.0%, 97.0%, 98.0%, 99.0%, and 100.0% homology or sequence identity to any one of the polypeptide or nucleic acid sequences disclosed herein.

Several homology or sequence identity searching programs based on polypeptide sequences are known in the art and can be used to identify molecules that are homologous. In one approach, a percent sequence identity can be determined by standard methods that are commonly used to compare the similarity and position of the amino acids of two polypeptides. Using a computer program such as BLAST or FASTA, two sequences can be aligned for optimal matching of their respective amino acids (either along the full length of one or both sequences, or along a predetermined portion of one or both sequences). Such programs provide “default” opening penalty and a “default” gap penalty, and a scoring matrix such as PAM 250 (a standard scoring matrix; see Dayhoff et al., in: Atlas of Protein Sequence and Structure, Vol. 5, Supp. 3 (1978)) can be used in conjunction with the computer program.

Similarly, the heterologous protein that may be included in the immunogenic compositions can be any heterologous protein that can be encoded in the nucleic acids within the immunogenic composition of nucleic acids discussed above, or those encoded in the isolated nucleic acids discussed above. Thus, in some embodiments, the heterologous protein is derived from a codon-optimized nucleic acid (e.g., SEQ. ID. No. 7 is derived from SEQ. ID. No. 8). The HBcAg may also be a native or variant form of the protein.

If the immunogenic composition includes a heterologous protein that is an HBV antigen, the heterologous protein can be substantially different than the HBcAg present in the immunogenic composition. As example, the immunogenic composition may include HBcAg, and a heterologous protein which is an HBV antigen that is non-naturally occurring or in a non-naturally occurring position with respect to the HBcAg. The immunogenic composition may include, for example, a mixture of SEQ. ID. No. 9 and 11, and therefore includes different HBV antigens (i.e., human HBcAg and heron HBcAg).

Non-limiting examples of admixtures of HBcAg, or a fragment thereof, and a heterologous protein, which may be included in the immunogenic compositions, include, but are not limited to: (1) stork HBcAg and HCV NS3/4A (e.g., SEQ. ID. Nos. 19 and 1); (2) heron HBcAg and HCV NS3/4A (e.g., SEQ. ID. Nos. 21 and 1); (3) stork HBcAg and HCV NS5A (e.g., SEQ. ID. Nos. 19 and 7); (4) heron HBcAg and HCV NS5A (e.g., SEQ. ID. Nos. 21 and 7); (5) stork HBcAg and human HBV e antigen (e.g., SEQ. ID. Nos. 19 and 11); (6) heron HBcAg and human HBV e antigen (e.g., SEQ. ID. Nos. 21 and 11); (7) stork HBcAg and human HBcAg (e.g., SEQ. ID. Nos. 19 and 9); (8) heron HBcAg and human HBcAg (e.g., SEQ. ID. Nos. 21 and 9); (9) stork HBcAg and birch antigen (e.g., SEQ. ID. Nos. 19 and 17); (10) heron HBcAg and birch antigen (e.g., SEQ. ID. Nos. 21 and 17); (11) stork HBcAg and ovalbumin antigen (e.g., SEQ. ID. Nos. 19 and 15); and (12) stork HBcAg and ovalbumin antigen (e.g., SEQ. ID. Nos. 21 and 15).

It is also contemplated that some immunogenic compositions can comprise both a protein as described herein and a nucleic acid as described herein. For example, some embodiments may include a nucleic acid encoding an HBcAg (e.g., a nucleic acid encoding a stork or heron HBcAg (e.g., SEQ. ID. No. 20 and 22) and a protein that is an antigen (e.g., HCV NS3/4A SEQ. ID. No. 1). Alternatively, some embodiments are immunogenic compositions that comprise an HBcAg protein (e.g., stork or heron HBcAg SEQ. ID. No. 19 and 21) and a nucleic acid encoding an antigen (e.g., a nucleic acid encoding HCV NS3/4A SEQ. ID. No. 2).

It is also contemplated that various other ingredients may be included to improve the immunogenic composition by, for example, increasing the immune response caused by the composition. Some embodiments of the composition may further include an adjuvant. Non-limiting example of adjuvants include interleukin-2 (IL2), interleukin-12 (IL12), interleukin-15 (IL15), interleukin-21 (IL21), interleukin-28b (IL28b), galactosyl transferase, a toll-like receptor (TLR), ribavirin, alum, CpGs, and an oil.

Various ingredients, such as excipients, adjuvants, binding agents, etc., may be included in the immunogenic compositions including a polypeptide. The same ingredients as those disclose above with respect to immunogenic compositions of isolated nucleic acids may be utilized.

### Immunogenic Compositions Comprising a Construct Expressing Flaviviral Envelope Proteins and a Construct Expressing Flaviviral Replicon and an Immunogen

Several embodiments provided herein are drawn to an immunogenic composition that includes a nucleic acid construct (e.g. plasmid) that expresses flaviviral structural proteins and a nucleic acid construct (e.g. plasmid) that expresses flaviviral non-structural replicon proteins and an immunogen. Without being bound by theory, it is contemplated that upon introduction to a subject, these constructs will produce “suicidal” flaviviral particles that can infect once and produce new non-structural replicon proteins and the immunogen. The suicidal particles can target professional antigen presenting cells such as Langerhan cells. Without being bound by theory, it is believed that the immunogenic compositions described herein can lower the amount of DNA for vaccination through the dual effect of (1) expressing the immunogen from the constructs and (2) replicating and amplifying the non-structural proteins and immunogen within the cells.

Flaviviruses that can be used in several embodiments of immunogenic compositions provided herein include but are not limited to West Nile virus, Dengue virus, Tick-borne encephalitis virus, Yellow fever virus, Aroa virus, Japanese encephalitis virus, Kokobera virus, Ntaya virus, Spondweni virus, Entebbe virus, Modoc virus, Rio Bravo virus, and the like. Additional non-limiting examples of flaviviruses contemplated for use herein include 58 species of the flavivirus genus for which 3,773 complete genomes are known and available at the Virus Pathogen Resource (ViPR) database (www.viprbrc.org).

Without being bound by theory, it is understood that the flavivirus genome encodes three structural proteins: the core (C), pre-membrane (pre-M), and envelope (E) proteins; and seven non-structural (NS) proteins in a single open reading frame. Additionally, the flavivirus genome includes 5′ and 3′ untranslated regions (UTRs) that are thought to have secondary structures involved in viral replication, translation, and packaging of the genomes. During assembly of flavivirus particles, structural proteins are inserted cotranslationally into the endoplasmic reticulum and processed by the NS proteins. The core proteins and genomic RNA are encapsidated by budding into the endoplasmic reticulum lumen and form the nucleocapsid.

Various embodiments are drawn to immunogenic compositions including a construct expressing tick-borne encephalitis virus structural proteins and a construct expressing tick-borne encephalitis virus non-structural replicon proteins and an immunogen. The tick-borne virus can be from any known subtype, including but not limited to: (a) Western European subtype (formerly Central European encephalitis virus, CEEV); (b) Siberian subtype (formerly West Siberian virus); and (c) Far Eastern subtype (formerly Russian Spring Summer encephalitis virus, RSSEV).

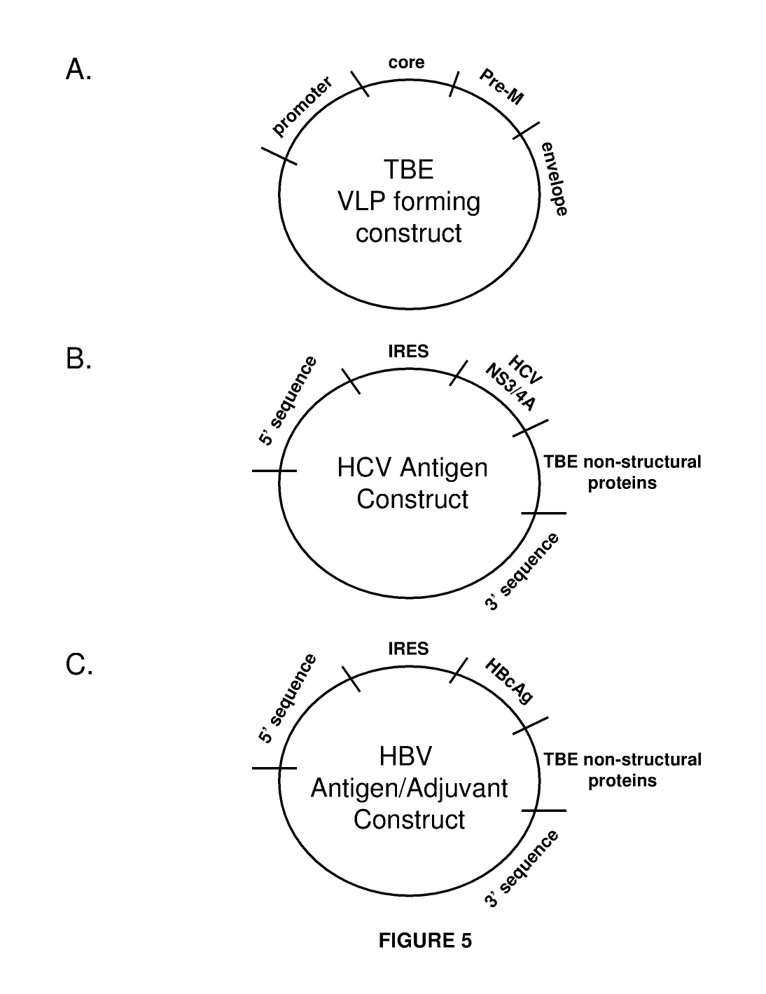

Several embodiments concern transfection of muscle or skin of a subject in need of a production of an immune response to an HCV and/or HBV infection with an immunogenic composition comprising an isolated first virus-like particle (VLP) forming construct (e.g. plasmid) comprising a nucleic acid sequence encoding a tick-borne encephalitis (TBE) core, Pre-M, and envelope proteins but lacking the TBE non-structural proteins (FIG. 5, plasmid A). In some embodiments, expression of these TBE proteins is driven by a constitutive promoter that is operably linked to the nucleic acids encoding said TBE core, Pre-M, and envelope proteins. In such an embodiment, the immunogenic composition also comprises a second construct, (e.g. an antigen plasmid) (FIG. 5, plasmid B), which can comprise a 5′ untranslated sequence, an IRES element, a nucleic acid encoding an antigen, TBE non-structural proteins and a 3′ untranslated sequence. In various embodiments, the antigen can include HCV NS3/4a (e.g. a codon-optimized NS3/4A sequence prepared as described herein).

Additionally, in several embodiments the immunogenic composition can include a third or more constructs (e.g. an antigen plasmid) (FIG. 5, plasmid C), which can comprise a 5′ untranslated sequence, an IRES element, a nucleic acid encoding an antigen that can be different from that of the second construct, TBE non-structural proteins and a 3′ untranslated sequence. In various embodiments, the antigen of the third or more constructs can include HBV core protein or HBcAg (e.g. a codon-optimized stork or heron HBcAg as described herein).

Referring to FIG. 5, it should be understood that the immunogenic composition can comprise the VLP forming plasmid (plasmid A) with either plasmid B or C or both plasmid B and C. As described herein, in some aspects of the invention, it is contemplated that plasmid C (the HBcAg-containing plasmid) will provide an adjuvant-like effect with respect to the immune response directed to NS3/4A when all three plasmids are administered. It should also be understood that the immunogenic composition can contain the VLP forming plasmid, and the antigen plasmids (e.g., plasmid B and/or C) in a single mixture such that the plasmids are coadministered, alternatively, the VLP forming plasmid, and the antigen plasmids (e.g., plasmid B and/or C) can be administered to said subject separately. If separate administration is performed, it is desired that the plasmids are administered at least or equal to about 1, 2, 3, 4, 5, 6, 7, 8, 9, 10, 15, 20, 30, 45, or one hour of one another. By design, the RNA expressed from the antigen plasmids (plasmid B and/or plasmid C) is then incorporated into the VLP generated by the TBE construct (plasmid A). Suicidal/defective VLPs containing this replicon RNA are released and the VLPs are allowed to infect Langerhan cells and dendritic cells, whereby a potent immune response in the subject is generated.

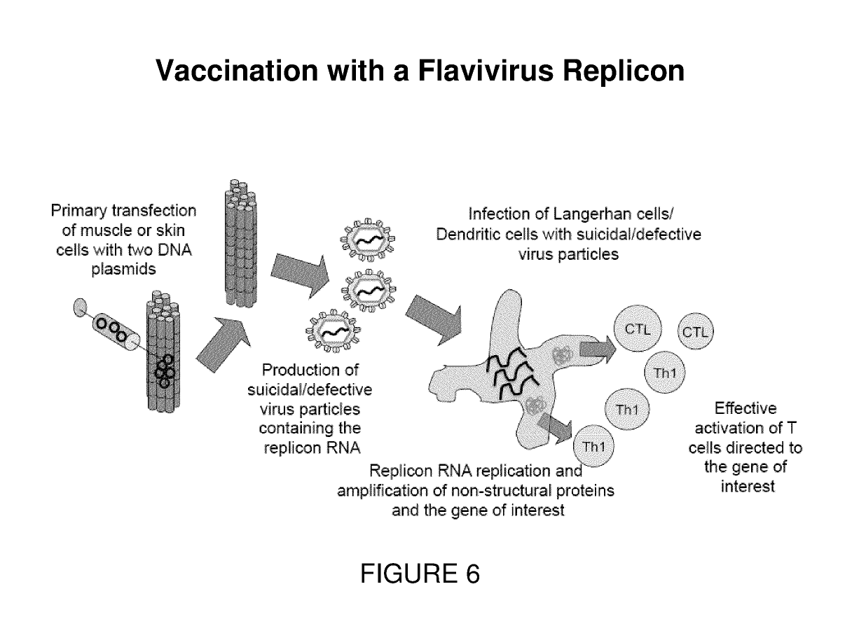

Referring to FIG. 6, it is contemplated in several embodiments that introduction of at least two plasmids (a VLP plasmid and one or more antigen plasmids) into the muscle or skin of a subject by injection, for example, leads to the production of suicidal flaviviral (e.g. TBE) particles that are infectious and harbor the replicon and immunogen that replicate in infected cells. However, the flaviviral (e.g. TBE) particles are capable of a single round of infection because the viral structural proteins are not encoded by the replicon. Consequently, the flaviviral (e.g. TBE) particles can infect antigen presenting cells (APCs) such as Langerhan cells or dendritic cells, leading to replicon RNA replication and amplification of non-structural proteins and the immunogen. It is considered that infection of APCs with the flaviviral (e.g. TBE) particles can induce effective activation of T cells directed to the immunogen.

Accordingly, several embodiments concern the use of the system described herein to promote an immune response in a subject in need thereof, for example, a subject that has been identified as having an HCV and/or HBV infection. Such individuals in need of an immune response to an HCV and/or HBV infection can be easily identified using clinical evaluation and readily available diagnostic tools known in the art.

### Immunogens for Use in a Construct Expressing Flaviviral Replicon

Various embodiments of DNA vaccines provided herein include a construct (e.g. plasmid) expressing non-structural flaviviral replicon proteins and an immunogen. Examples of suitable immunogens include but are not limited to plant antigen (e.g., birch antigen), viral antigen, or an animal antigen (e.g., ovalbumin antigen). The immunogen may also be a hepatitis antigen, for example a hepatitis B virus (HBV) antigen or a hepatitis C virus (HCV) antigen. The HCV antigens can be from viruses known to infect animals of any species, including, but not limited to, amphibians, reptiles, birds (e.g., stork and heron) mice, hamsters, rats, rabbits, guinea pigs, woodchucks, pigs, micro-pigs, goats, dogs, cats, humans and non-human primates (e.g., baboons, monkeys, and chimpanzees). Similarly, the HBV antigens can be from viruses known to infect animals of any species, including, but not limited to, amphibians, reptiles, birds (e.g., stork and heron), and heron, mice, hamsters, rodents, pigs, micro-pigs, goats, dogs, cats, humans and non-human primates (e.g., baboons, monkeys, and chimpanzees). In certain embodiments, the antigen is a HCV antigen selected from NS3/4A, NS5A, and combinations thereof. In certain embodiments, the antigen is a HBV antigen selected from a HBV surface antigen, HBV e antigen, human HBcAg, a human HBV polymerase antigen, a human HBV x antigen, and combinations thereof. Any of the antigens described herein can be used as suitable immunogens in various embodiments.

### Codon Optimization

Various embodiments include nucleic acid sequences encoding immunogens that are codon optimized for expression in humans. As used herein, the term “codon-optimized” means a nucleic acid coding region that has been adapted for expression in the cells of a given organism by replacing at least one, or more than one, or a significant number, of codons with one or more codons that are more frequently used in the genes of that organism.

Deviations in the nucleotide sequence that comprise the codons encoding the amino acids of any polypeptide chain allow for variations in the sequence coding for the gene. Since each codon consists of three nucleotides, and the nucleotides comprising DNA are restricted to four specific bases, there are 64 possible combinations of nucleotides, 61 of which encode amino acids (the remaining three codons encode stop signals ending translation). The “genetic code” table is reproduced in Table 1 below.

TABLE 1
The Standard Genetic Code

| | T | C | A | G |
|---|---|---|---|---|
| T | TTT Phe | TCT Ser | TAT Tyr (Y) | TGT Cys (C) |
| | (F) | (C) | | |
| | TTC | TCC | TAC | TGC |
| | TTA Leu | TCA | TAA (Stop) | TGA (Stop) |
| | (L) | | | |
| | TTG | TCG | TAG (Stop) | TGG Trp (W) |
| C | CTT Leu | CCT Pro | CAT His (H) | CGT Arg (R) |
| | (L) | (P) | | |
| | CTC | CCC | CAC | CGC |
| | CTA | CCA | CAA Gln (Q) | CGA |
| | CTG | CCG | CAG | CGG |
| A | ATT Ile | ACT Thr | AAT Asn (N) | AGT Ser (S) |
| | (I) | (T) | | |
| | ATC | ACC | AAC | AGC |
| | ATA | ACA | AAA Lys (K) | AGA Arg (R) |
| | ATG Met | ACG | AAG | AGG |
| | (M) | | | |
| G | GTT Val | GCT Ala | GAT Asp (D) | GGT Gly (G) |
| | (V) | (A) | | |
| | GTC | GCC | GAC | GGC |
| | GTA | GCA | GAA Glu (E) | GGA |
| | GTG | GCG | GAG | GGG |

The genetic code table indicates that many amino acids are designated by more than one codon. For example, the amino acids alanine and proline are coded for by four triplets, serine and arginine by six, whereas tryptophan and methionine are coded by just one triplet. This degeneracy allows for DNA base composition to vary over a wide range without altering the amino acid sequence of the proteins encoded by the DNA. Many organisms display a bias for use of particular codons to code for insertion of a particular amino acid in a growing peptide chain. Codon preference or codon bias, differences in codon usage between organisms, is afforded by degeneracy of the genetic code, and is well documented among many organisms. Codon bias often correlates with the efficiency of translation of messenger RNA (mRNA), which is in turn believed to be dependent on, inter alia, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

Given the large number of gene sequences available for a wide variety of animal, plant and microbial species, it is possible to calculate the relative frequencies of codon usage. Codon usage tables are readily available, for example, at the “Codon Usage Database” available at www.kazusa.orjp/codon. The human codon usage table calculated from GenBank is reproduced below in Table 2, which uses mRNA nomenclature. Accordingly, Table 2 uses uracil (U), which is found in RNA, instead of thymine (T), which is found in DNA.

TABLE 2
Codon Usage Table for Human Genes

| Amino Acid | Codon | Number | Frequency |
|---|---|---|---|
| Phe | UUU | 326146 | 0.4525 |
| Phe | UUC | 394680 | 0.5475 |
| Total | | 720826 | |
| Leu | UUA | 139249 | 0.0728 |
| Leu | UUG | 242151 | 0.1266 |
| Leu | CUU | 246206 | 0.1287 |
| Leu | CUC | 374262 | 0.1956 |
| Leu | CUA | 133980 | 0.07 |
| Leu | CUG | 777077 | 0.4062 |
| Total | | 1912925 | |
| Ile | AUU | 303721 | 0.3554 |
| Ile | AUC | 414483 | 0.485 |
| Ile | AUA | 136399 | 0.1596 |
| Total | | 854603 | |
| Met | AUG | 430946 | 1 |
| Total | | 430946 | |
| Val | GUU | 210423 | 0.1773 |
| Val | GUC | 282445 | 0.238 |
| Val | GUA | 134991 | 0.1137 |
| Val | GUG | 559044 | 0.471 |
| Total | | 1186903 | |
| Ser | UCU | 282407 | 0.184 |
| Ser | UCC | 336349 | 0.2191 |
| Ser | UCA | 225963 | 0.1472 |
| Ser | UCG | 86761 | 0.0565 |
| Ser | AGU | 230047 | 0.1499 |
| Ser | AGC | 373362 | 0.2433 |
| Total | | 1534889 | |
| Pro | CCU | 333705 | 0.2834 |
| Pro | CCC | 386462 | 0.3281 |
| Pro | CCA | 322220 | 0.2736 |
| Pro | CCG | 135317 | 0.1149 |
| Total | | 1177704 | |
| Thr | ACU | 247913 | 0.2419 |
| Thr | ACC | 371420 | 0.3624 |
| Thr | ACA | 285655 | 0.2787 |
| Thr | ACG | 120022 | 0.1171 |
| Total | | 1025010 | |
| Ala | GCU | 360146 | 0.2637 |
| Ala | GCC | 551452 | 0.4037 |
| Ala | GCA | 308034 | 0.2255 |
| Ala | GCG | 146233 | 0.1071 |
| Total | | 1365865 | |
| Tyr | UAU | 232240 | 0.4347 |
| Tyr | UAC | 301978 | 0.5653 |
| Total | | 534218 | |
| His | CAU | 201389 | 0.4113 |
| His | CAC | 288200 | 0.5887 |
| Total | | 489589 | |
| Gln | CAA | 227742 | 0.2541 |
| Gln | CAG | 668391 | 0.7459 |
| Total | | 896133 | |
| Asn | AAU | 322271 | 0.4614 |
| Asn | AAC | 376210 | 0.5386 |
| Total | | 698481 | |
| Lys | AAA | 462660 | 0.4212 |
| Lys | AAG | 635755 | 0.5788 |
| Total | | 1098415 | |
| Asp | GAU | 430744 | 0.4613 |
| Asp | GAC | 502940 | 0.5387 |
| Total | | 933684 | |
| Glu | GAA | 561277 | 0.4161 |
| Glu | GAG | 787712 | 0.5839 |
| Total | | 1348989 | |
| Cys | UGU | 190962 | 0.4468 |
| Cys | UGC | 236400 | 0.5532 |
| Total | | 427362 | |
| Trp | UGG | 248083 | 1 |
| Total | | 248083 | |
| Arg | CGU | 90899 | 0.083 |
| Arg | CGC | 210931 | 0.1927 |
| Arg | CGA | 122555 | 0.112 |
| Arg | CGG | 228970 | 0.2092 |
| Arg | AGA | 221221 | 0.2021 |
| Arg | AGG | 220119 | 0.2011 |
| Total | | 1094695 | |
| Gly | GGU | 209450 | 0.1632 |
| Gly | GGC | 441320 | 0.3438 |
| Gly | GGA | 315726 | 0.2459 |
| Gly | GGG | 317263 | 0.2471 |
| Total | | 1283759 | |
| Stop | UAA | 13963 | |
| Stop | UAG | 10631 | |
| Stop | UGA | 24607 | |

By utilizing this or similar tables, one of ordinary skill in the art can apply the frequencies to any given polypeptide sequence, and produce a nucleic acid fragment of a codon-optimized coding region which encodes the polypeptide, but which uses codons more optimal for a given species. Codon-optimized coding regions can be designed by various different methods.

As an example, in one method termed “uniform optimization,” a codon usage table is used to find the single most frequent codon used for any given amino acid, and that codon is used each time that particular amino acid appears in the polypeptide sequence.

As another example, in a method termed “full-optimization,” the actual frequencies of the codons are distributed randomly throughout the coding region. Thus, using this method for optimization, if a hypothetical polypeptide sequence had 100 leucine residues, referring to Table 2 for frequency of usage in the humans, about 7, or 7% of the leucine codons would be UUA, about 13, or 13% of the leucine codons would be UUG, about 13, or 13% of the leucine codons would be CUU, about 20, or 20% of the leucine codons would be CUC, about 7, or 7% of the leucine codons would be CUA, and about 41, or 41% of the leucine codons would be CUG. These frequencies would be distributed randomly throughout the leucine codons in the coding region encoding the hypothetical polypeptide. As will be understood by those of ordinary skill in the art, the distribution of codons in the sequence can vary significantly using this approach, however, the sequence always encodes the same polypeptide.

As a further example, in a method termed “minimal optimization,” coding regions are only partially optimized. For example, the invention includes a nucleic acid fragment of a codon-optimized coding region encoding a polypeptide in which at least about 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or 100% of the codon positions have been codon-optimized for a given species. That is, they contain a codon that is preferentially used in the genes of a desired species, e.g., a vertebrate species, e.g., humans, in place of a codon that is normally used in the native nucleic acid sequence. Codons that are rarely found in human genes are changed to codons more commonly utilized in human coding regions.

The above-described methods of codon optimization are non-limiting examples of identifying, selecting, and/or preparing codon-optimized immunogens for use in several embodiments of immunogenic compositions provided herein.

### Methods of Enhancing or Promoting an Immune Response

Methods of enhancing or promoting an immune response in an animal, including humans, to an antigen are also provided. Such methods can be practiced, for example, by identifying an animal in need of an immune response and administering said animal with any of the immunogenic compositions described above that is effective to enhance or facilitate an immune response to the heterologous protein. In some embodiments, compositions including one or more isolated nucleic acids encoding the HBcAg antigen, or a fragment thereof, and a nucleic acid encoding a heterologous protein are administered to a animal in need thereof at the same time in the same mixture. In certain embodiments, compositions of HBcAg antigen, or a fragment thereof, and a heterologous protein are administered to the animal at the same time in the same mixture. Alternatively, the nucleic acid encoding the HBcAg and the nucleic acid encoding the heterologous protein are coadministered. Similarly, the HBcAg protein and the protein antigen can be coadministered. By coadministered, it is mean that the two nucleic acids or two protein are provided at the same time in the same mixture or within at least, equal to, or about any number in between 1, 5, 10, 15, 20, 30, 40, 50, or 60 minutes each separate administration. However, the present invention is not limited to any particular order of administration.

Accordingly, some methods include administering a composition comprising an isolated nucleic acid encoding HBcAg, or a fragment thereof, joined to an isolated nucleic acid encoding a heterologous protein. Non-limiting examples of compositions that may be administered according to the methods disclosed herein include, but are not limited t nucleic acids encoding: (1) stork HBcAg joined to HCV NS3/4A (e.g., SEQ. ID. No. 24 and 26); (2) heron HBcAg joined to HCV NS3/4A (e.g., SEQ. ID. No. 36); (3) stork HBcAg joined to HCV NS5A (e.g., SEQ. ID. No. 40); (4) heron HBcAg joined to HCV NS5A (e.g., SEQ. ID. No. 42); (5) stork HBcAg joined to human HBV e antigen (e.g., SEQ. ID. No. 44 and 46); (6) heron HBcAg joined to human HBV e antigen (e.g., SEQ. ID. No. 48 and 50); (7) stork HBcAg and human HBcAg (e.g., SEQ. ID. No. 52 and 103); (8) heron HBcAg joined to human HBcAg (e.g., SEQ. ID. No. 50 and 105); (9) stork HBcAg joined to birch antigen (e.g., SEQ. ID. No. 56); (10) heron HBcAg joined to birch antigen (e.g., SEQ. ID. No. 58); (11) stork HBcAg joined to ovalbumin antigen (e.g., SEQ. ID. No. 60); and (12) stork HBcAg joined to ovalbumin antigen (e.g., SEQ. ID. No. 62).

Furthermore, compositions including nucleic acid sequences encoding HBcAg, or a fragment thereof, and nucleic acid sequences encoding a heterologous protein in Trans, may be administered according to the methods disclosed herein. Non-limiting examples of compositions for administering according to the methods disclosed herein, include, but are not limited to nucleic acids encoding: (1) stork HBcAg and HCV NS3/4A (e.g., SEQ. ID. Nos. 20 and 2); (2) heron HBcAg and HCV NS3/4A (e.g., SEQ. ID. Nos. 22 and 2); (3) stork HBcAg and HCV NS5A (e.g., SEQ. ID. Nos. 20 and 8); (4) heron HBcAg and HCV NS5A (e.g., SEQ. ID. Nos. 22 and 8); (5) stork HBcAg and human HBV e antigen (e.g., SEQ. ID. Nos. 20 and 12); (6) heron HBcAg and human HBV e antigen (e.g., SEQ. ID. Nos. 22 and 12); (7) stork HBcAg and human HBcAg (e.g., SEQ. ID. Nos. 20 and 10); (8) heron HBcAg and human HBcAg (e.g., SEQ. ID. Nos. 22 and 10); (9) stork HBcAg and birch antigen (e.g., SEQ. ID. Nos. 20 and 18); (10) heron HBcAg and birch antigen (e.g., SEQ. ID. Nos. 22 and 18); (11) stork HBcAg and ovalbumin antigen (e.g., SEQ. ID. Nos. 20 and 16); and (12) stork HBcAg and ovalbumin antigen (e.g., SEQ. ID. Nos. 22 and 16).

In addition, compositions including HBcAg, or a fragment thereof, and a heterologous protein, may be administered according to the methods disclosed herein. Non-limiting examples of the compositions for administering according to the methods disclosed herein, include, but are not limited to: (1) stork HBcAg and HCV NS3/4A (e.g., SEQ. ID. Nos. 19 and 1); (2) heron HBcAg and HCV NS3/4A (e.g., SEQ. ID. Nos. 21 and 1); (3) stork HBcAg and HCV NS5A (e.g., SEQ. ID. Nos. 19 and 7); (4) heron HBcAg and HCV NS5A (e.g., SEQ. ID. Nos. 21 and 7); (5) stork HBcAg and human HBV e antigen (e.g., SEQ. ID. Nos. 19 and 11); (6) heron HBcAg and human HBV e antigen (e.g., SEQ. ID. Nos. 21 and 11); (7) stork HBcAg and human HBcAg (e.g., SEQ. ID. Nos. 19 and 9); (8) heron HBcAg and human HBcAg (e.g., SEQ. ID. Nos. 21 and 9); (9) stork HBcAg and birch antigen (e.g., SEQ. ID. Nos. 19 and 17); (10) heron HBcAg and birch antigen (e.g., SEQ. ID. Nos. 21 and 17); (11) stork HBcAg and ovalbumin antigen (e.g., SEQ. ID. Nos. 19 and 15); and (12) stork HBcAg and ovalbumin antigen (e.g., SEQ. ID. Nos. 21 and 15).

Other embodiments concern methods of inhibiting HCV infection, reducing HCV viral titer, inhibiting HCV replication, treating HCV infection or promoting an immune response specific for an HCV protein. By one approach, an immunogenic composition comprising an isolated nucleic acid encoding HBcAg, or a fragment thereof (e.g., a human codon-optimized nucleic acid encoding a HBcAg derived from an avian hepatitis, such as a hepatitis that infects stork (e.g., SEQ. ID. No. 20) or heron (e.g., SEQ. ID. No. 22)) and an isolated nucleic acid encoding an HCV antigen described herein (e.g., a human codon-optimized nucleic acid encoding NS3, NS3/4A (e.g., SEQ. ID. No. 2), and/or NS5A (e.g., SEQ. ID. 8) are used to prepare a medicament for the inhibition of HCV infection, the reduction of HCV viral titer, the inhibition of HCV replication, the treatment of HCV infection or for the generation of an immune response to an HCV protein. That is, preferred compositions comprise, consist essentially of, or consist of a nucleic acid encoding HBcAg derived from an avian hepatitis (e.g., SEQ. ID. Nos. 20 and 22) and a nucleic acid encoding an HCV protein derived from a hepatitis virus that infects humans (e.g., SEQ. ID. No. 2). The nucleic acids present in said compositions can be in C is (e.g., operably joined in frame) or in Trans (e.g., on separate expression constructs altogether). By one approach, an individual in need of a medicament that inhibits HCV infection, reduces HCV viral titer, inhibits HCV replication, treats HCV infection or that promotes an immune response to an HCV protein is identified and said individual is provided a medicament comprising a nucleic acid encoding an HBcAg antigen (e.g., SEQ. ID. Nos. 20 and 22) and a nucleic acid encoding an HCV antigen, such as codon-optimized NS3/4A (e.g., SEQ. ID. NO.: 1), or codon-optimized NS5A (e.g., SEQ. ID. No. 8).

Alternatively, an immunogenic composition comprising an HBcAg polypeptide (e.g., SEQ. ID. Nos. 21 and 23), or a fragment thereof, and an HCV antigen described herein (e.g., codon-optimized NS3/4A in SEQ. ID. No. 1) are used to prepare a medicament for the inhibition of HCV infection, the reduction of HCV viral titer, the inhibition of HCV replication, the treatment of HCV infection or for the generation of an immune response to an HCV protein.

Some embodiments concern methods of inhibiting HBV infection, reducing HBV viral titer, inhibiting HBV replication, treating HBV infection or promoting an immune response specific for an HBV protein. By one approach, an immunogenic composition comprising a nucleic acid encoding HBcAg (e.g., a human codon-optimized nucleic acid encoding a HBcAg derived from an avian hepatitis, such as a hepatitis that infects stork (e.g., SEQ. ID. No. 20) or heron (e.g., SEQ. ID. No. 22)) and an isolated nucleic acid encoding an HBV antigen described herein (e.g., a human codon-optimized nucleic acid encoding a HBcAg (e.g., SEQ. ID. No. 10), a HBV surface antigen, a HBV e antigen (e.g., SEQ. ID. Nos. 12 and 14), a HBV polymerase antigen, or a HBV x antigen derived from a hepatitis that infects humans) are used to prepare a medicament for the inhibition of HBV infection, the reduction of HBV viral titer, the inhibition of HBV replication, the treatment of HBV infection or for the generation of an immune response to an HBV protein. That is, preferred compositions comprise, consist essentially of, or consist of an HBcAg derived from an avian hepatitis and a nucleic acid encoding an HBV protein derived from a hepatitis virus that infects humans. The nucleic acids present in said compositions can be in C is (e.g., operably joined in frame) or in Trans (e.g., on separate expression constructs altogether). By one approach, an individual in need of a medicament that inhibits HBV infection, reduces HBV viral titer, inhibits HBV replication, treats HBV infection or that promotes an immune response to an HBV protein is identified and said individual is provided a medicament comprising a nucleic acid encoding an avian HBcAg (e.g., SEQ. ID. No. 20 and 22) and an HBV antigen, such as codon-optimized HBV antigen (e.g., codon-optimized HBcAg (e.g., SEQ. ID. NO.: 11)).

Alternatively, an immunogenic composition comprising an HBcAg polypeptide, or a fragment thereof, and an HBV antigen described herein are used to prepare a medicament for the inhibition of HBV infection, the reduction of HBV viral titer, the inhibition of HBV replication, the treatment of HBV infection or for the generation of an immune response to an HBV protein.

Some embodiments concern methods of ameliorating a birch allergy, reducing sensitivity to a birch allergen, or reducing IgE antibody levels specific to birch. By one approach, an immunogenic composition comprising a nucleic acid encoding HBcAg (e.g., a human codon-optimized nucleic acid encoding a HBcAg derived from an avian hepatitis, such as a hepatitis that infects stork (e.g., SEQ. ID. No. 20) or heron (e.g., SEQ. ID. No. 22)) and an isolated nucleic acid encoding a birch antigen (e.g., SEQ. ID. No 18) are used to prepare a medicament for the ameliorating a birch allergy, reducing sensitivity to a birch allergy, or reducing IgE antibody levels specific to birch. That is, preferred compositions comprise, consist essentially of, or consist of an HBcAg derived from an avian hepatitis and a nucleic acid encoding an birch antigen derived. The nucleic acids present in said compositions can be in C is (e.g., operably joined in frame) or in Trans (e.g., on separate expression constructs altogether). By one approach, an individual in need of a medicament that ameliorates a birch allergy, reduces sensitivity to a birch allergen, or reduces IgE antibody levels specific to birch is identified and said individual is provided a medicament comprising a nucleic acid encoding an avian HBcAg (e.g., SEQ. ID. No. 20 and 22) and a birch antigen, such as codon-optimized birch antigen (e.g., SEQ. ID. NO.: 18).

Alternatively, an immunogenic composition comprising an HBcAg polypeptide, or a fragment thereof, and an birch antigen described herein are used to prepare a medicament for ameliorating a birch allergy, reducing sensitivity to a birch allergen, or reducing IgE antibody levels specific to birch antigen.

The effective dose and method of administration of a particular formulation can vary based on the individual patient and the type and stage of the disease, as well as other factors known to those of skill in the art. Therapeutic efficacy and toxicity can be determined by standard pharmaceutical procedures in cell cultures or experimental animals, e.g., ED50 (the dose therapeutically effective in 50% of the population). The data obtained from cell culture assays and animal studies can be used to formulate a range of dosage for human use. The dosage lies preferably within a range of circulating concentrations that include the ED50 with no toxicity. The dosage varies within this range depending upon the type of adjuvant derivative and antigen, the dosage form employed, the sensitivity of the patient, and the route of administration.

In certain embodiments an adjuvant is included within the administered composition. For instance, a pharmacologic agent can be added to a composition described herein as needed to increase or aid its effect. In another example, an immunological agent that increases the antigenic response can be utilized with a device described herein. For instance, U.S. Pat. No. 6,680,059 (which is hereby incorporated in its entirety by reference) describes the use of vaccines containing ribavirin as an adjuvant to the vaccine. However, an adjuvant may refer to any material that has the ability to enhance or facilitate an immune response or to increase or aid the effect of a therapeutic agent. Non-limiting example of adjuvants include interleukin-2 (IL2), interleukin-12 (IL12), interleukin-15 (IL15), interleukin-21 (IL21), interleukin-28b (IL28b), galactosyl transferase, a toll-like receptor (TLR), ribavirin, alum, CpGs, and an oil. Also, as described above, in some embodiments, the composition includes an isolated nucleic acid, or constructs comprising said nucleic acids, encoding a protein that is an adjuvant, such as IL2, IL12, IL15, IL21, IL28b, galactosyl transferase, a TLR, and the like. In certain aspects, the isolated nucleic acid encoding the protein which is an adjuvant may be in the same construct encoding HBcAg and/or the heterologous protein. In some aspects, methods of administering the immunogenic composition comprise administering an adjuvant before administering the immunogenic composition.

In some embodiments, the method includes administering an immunogenic composition that comprises an isolated nucleic that encodes HBcAg, or a fragment thereof, and separately administering an isolated nucleic acid that encodes a heterologous protein (e.g., SEQ. ID. No. 8). When the isolated nucleic acid encoding HBcAg and the isolated nucleic acid encoding the heterologous protein are administered separately, the isolated nucleic acid encoding HBcAg may, in some embodiments, may be administered before the isolated nucleic acid encoding heterologous protein. Alternatively, the isolated nucleic acid encoding heterologous protein may, in some embodiments, be administered before the isolated nucleic acid encoding HBcAg.

Other embodiments of the methods disclosed herein include administering a composition including both HBcAg and the heterologous protein. In some embodiments, the method includes administering an immunogenic composition that includes an admixture of an isolated nucleic acid encoding HBcAg and an isolated nucleic acid encoding the heterologous protein. In certain embodiments, the method includes administering an immunogenic composition that includes an admixture of an isolated nucleic acid encoding the HBcAg and an isolated nucleic acid encoding the heterologous protein.

Various routes of administration may be used for the methods described herein. In some embodiments, the immunogenic composition is administered parenterally (e.g., intramuscularly, intraperitoneally, subcutaneously, or intravenously to a mammal subject). In a preferred embodiment, the immunogenic compositions are administered intramuscularly, dermally, or subcutaneously. The methods may also include applying electrical stimulation, which can enhance the administration of the immunogenic compositions. As an example, electroporation may be included in the present methods disclosed herein. Electroporation includes applying electrical stimulation to improve the permeability of cells to the administered composition. Examples of electroporation techniques are disclosed in U.S. Pat. Nos. 6,610,044 and 5,273,525, the disclosures of both of these references are hereby incorporated by reference in their entireties.

The concentration of the nucleic acid or protein in the immunogenic composition to be administered can vary from about 0.1 ng/ml to about 50 mg/ml. In some aspects, the concentration of the immunogenic composition administered (e.g., a suitable dose of nucleic acid or protein for administration) is between about 10 ng/ml to 25 mg/ml. In still other aspects, the concentration is between 100 ng/ml to 10 mg/ml. In some aspects, the suitable dose of nucleic acid or protein for administration is greater than or equal to or less than about 100 ng/ml, 150 ng/ml, 200 ng/ml, 250 ng/ml, 300 ng/ml, 350 ng/ml, 400 ng/ml, 450 ng/ml, 500 ng/ml, 550 ng/ml, 600 ng/ml, 650 ng/ml, 700 ng/ml, 750 ng/ml, 800 ng/ml, 850 ng/ml, 900 ng/ml, 950 ng/ml, 1 μg/ml, 2 μg/ml, 3 μg/ml, 4 μg/ml, 5 μg/ml, 6 μg/ml, 7 μg/ml, 8 μg/ml, 9 μg/ml, 10 μg/ml, 11 μg/ml, 12 μg/ml, 13 μg/ml, 14 μg/ml, 15 μg/ml, 16 μg/ml, 17 μg/ml, 18 μg/ml, 19 μg/ml, 20 μg/ml, 21 μg/ml, 22 μg/ml, 23 μg/ml, 24 μg/ml, 25 μg/ml, 26 μg/ml, 27 μg/ml, 28 μg/ml, 29 μg/ml, 30 μg/ml, 31 μg/ml, 32 μg/ml, 33 μg/ml, 34 μg/ml, 35 μg/ml, 36 μg/ml, 37 μg/ml, 38 μg/ml, 39 μg/ml, 40 μg/ml, 41 μg/ml, 42 μg/ml, 43 μg/ml, 44 μg/ml, 45 μg/ml, 46 μg/ml, 47 μg/ml, 48 μg/ml, 49 μg/ml, 50 μg/ml, 55 μg/ml, 60 μg/ml, 65 μg/ml, 70 μg/ml, 75 μg/ml, 80 μg/ml, 85 μg/ml, 90 μg/ml, 95 μg/ml, 100 μg/ml, 150 μg/ml, 200 μg/ml, 250 μg/ml, 300 μg/ml, 350 μg/ml, 400 μg/ml, 450 μg/ml, 500 μg/ml, 550 μg/ml, 600 μg/ml, 650 μg/ml, 700 μg/ml, 750 μg/ml, 800 μg/ml, 850 μg/ml, 900 μg/ml, 950 μg/ml, 1.0 mg/ml, 1.1 mg/ml, 1.2 mg/ml, 1.3 mg/ml, 1.4 mg/ml, 1.5 mg/ml, 1.6 mg/ml, 1.7 mg/ml, 1.8 mg/ml, 1.9 mg/ml, 2.0 mg/ml, 2.1 mg/ml, 2.2 mg/ml, 2.3 mg/ml, 2.4 mg/ml, 2.5 mg/ml, 2.6 mg/ml, 2.7 mg/ml, 2.8 mg/ml, 2.9 mg/ml, 3.0 mg/ml, 3.1 mg/ml, 3.2 mg/ml, 3.3 mg/ml, 3.4 mg/ml, 3.5 mg/ml, 3.6 mg/ml, 3.7 mg/ml, 3.8 mg/ml, 3.9 mg/ml, 4.0 mg/ml, 4.1 mg/ml, 4.2 mg/ml, 4.3 mg/ml, 4.4 mg/ml, 4.5 mg/ml, 4.6 mg/ml, 4.7 mg/ml, 4.8 mg/ml, 4.9 mg/ml, 5.0 mg/ml, 5.1 mg/ml, 5.2 mg/ml, 5.3 mg/ml, 5.4 mg/ml, 5.5 mg/ml, 5.6 mg/ml, 5.7 mg/ml, 5.8 mg/ml, 5.9 mg/ml, 6.0 mg/ml, 6.1 mg/ml, 6.2 mg/ml, 6.3 mg/ml, 6.4 mg/ml, 6.5 mg/ml, 6.6 mg/ml, 6.7 mg/ml, 6.8 mg/ml, 6.9 mg/ml, 7.0 mg/ml, 7.1 mg/ml, 7.2 mg/ml, 7.3 mg/ml, 7.4 mg/ml, 7.5 mg/ml, 7.6 mg/ml, 7.7 mg/ml, 7.8 mg/ml, 7.9 mg/ml, 8.0 mg/ml, 8.1 mg/ml, 8.2 mg/ml, 8.3 mg/ml, 8.4 mg/ml, 8.5 mg/ml, 8.6 mg/ml, 8.7 mg/ml, 8.8 mg/ml, 8.9 mg/ml, 9.0 mg/ml, 9.1 mg/ml, 9.2 mg/ml, 9.3 mg/ml, 9.4 mg/ml, 9.5 mg/ml, 9.6 mg/ml, 9.7 mg/ml, 9.8 mg/ml, 9.9 mg/ml, 10.0 mg/ml, 11 mg/ml, 12 mg/ml, 13 mg/ml, 14 mg/ml, 15 mg/ml, 16 mg/ml, 17 mg/ml, 18 mg/ml, 19 mg/ml, 20 mg/ml, 21 mg/ml, 22 mg/ml, 23 mg/ml, 24 mg/ml, 25 mg/ml, 26 mg/ml, 27 mg/ml, 28 mg/ml, 29 mg/ml, 30 mg/ml, 31 mg/ml, 32 mg/ml, 33 mg/ml, 34 mg/ml, 35 mg/ml, 36 mg/ml, 37 mg/ml, 38 mg/ml, 39 mg/ml, 40 mg/ml, 41 mg/ml, 42 mg/ml, 43 mg/ml, 44 mg/ml, 45 mg/ml, 46 mg/ml, 47 mg/ml, 48 mg/ml, 49 mg/ml, 50 mg/ml, or within a range defined by, and including, any two of these values.

The amount of nucleic acid or protein administered using the methods described herein can vary from about 1 ng to 10 g. In some aspects, the amount of nucleic acid or protein contained administered is less than greater than or equal to about 1 ng, 5 ng, 10 ng, 20 ng, 30 ng, 40 ng, 50 ng, 60 ng, 70 ng, 80 ng, 90 ng, 100 ng, 150 ng, 200 ng, 250 ng, 300 ng, 350 ng, 400 ng, 500 ng, 600 ng, 700 ng, 800 ng, 900 ng, 1 μg 1 μg, 2 μg, 3 μg, 4 μg, 5 μg, 6 μg, 7 μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg 150 μg, 155 μg, 160 μg, 165 μg, 170 μg, 175 μg, 180 μg, 185 μg, 190 μg, 195 μg 200 μg, 205 μg, 210 μg, 215 μg, 220 μg, 225 μg, 230 μg, 235 μg, 240 μg, 245 μg 250 μg, 255 μg, 260 μg, 265 μg, 270 μg, 275 μg, 280 μg, 285 μg, 290 μg, 295 μg, 300 μg, 305 μg, 310 μg, 315 μg, 320 μg, 325 μg, 330 μg, 335 μg, 340 μg, 345 μg 350 μg, 355 μg, 360 μg, 365 μg, 370 μg, 375 μg, 380 μg, 385 μg, 390 μg, 395 μg 400 μg, 405 μg, 410 μg, 415 μg, 420 μg, 425 μg, 430 μg, 435 μg, 440 μg, 445 μg 450 μg, 455 μg, 460 μg, 465 μg, 470 μg, 475 μg, 480 μg, 485 μg, 490 μg, 495 μg 500 μg, 505 μg, 510 μg, 515 μg, 520 μg, 525 μg, 530 μg, 535 μg, 540 μg, 545 μg 550 μg, 555 μg, 560 μg, 565 μg, 570 μg, 575 μg, 580 μg, 585 μg, 590 μg, 595 μg 600 μg, 605 μg, 610 μg, 615 μg, 620 μg, 625 μg, 630 μg, 635 μg, 640 μg, 645 μg 650 μg, 655 μg, 660 μg, 665 μg, 670 μg, 675 μg, 680 μg, 685 μg, 690 μg, 695 μg, 700 μg, 705 μg, 710 μg, 715 μg, 720 μg, 725 μg, 730 μg, 735 μg, 740 μg, 745 μg 750 μg, 755 μg, 760 μg, 765 μg, 770 μg, 775 μg, 780 μg, 785 μg, 790 μg, 795 μg, 800 μg, 805 μg, 810 μg, 815 μg, 820 μg, 825 μg, 830 μg, 835 μg, 840 μg, 845 μg 850 μg, 855 μg, 860 μg, 865 μg, 870 μg, 875 μg, 880 μg, 885 μg, 890 μg, 895 μg 900 μg, 905 μg, 910 μg, 915 μg, 920 μg, 925 μg, 930 μg, 935 μg, 940 μg, 945 μg 950 μg, 955 μg, 960 μg, 965 μg, 970 μg, 975 μg, 980 μg, 985 μg, 990 μg, 995 μg, 1.0 mg, 1.1 mg, 1.2 mg, 1.3 mg, 1.4 mg, 1.5 mg, 1.6 mg, 1.7 mg, 1.8 mg, 1.9 mg, 2.0 mg, 2.1 mg, 2.2 mg, 2.3 mg, 2.4 mg, 2.5 mg, 2.6 mg, 2.7 mg, 2.8 mg, 2.9 mg, 3.0 mg, 3.1 mg, 3.2 mg, 3.3 mg, 3.4 mg, 3.5 mg, 3.6 mg, 3.7 mg, 3.8 mg, 3.9 mg, 4.0 mg, 4.1 mg, 4.2 mg, 4.3 mg, 4.4 mg, 4.5 mg, 4.6 mg, 4.7 mg, 4.8 mg, 4.9 mg, 5.0 mg, 5.1 mg, 5.2 mg, 5.3 mg, 5.4 mg, 5.5 mg, 5.6 mg, 5.7 mg, 5.8 mg, 5.9 mg, 6.0 mg, 6.1 mg, 6.2 mg, 6.3 mg, 6.4 mg, 6.5 mg, 6.6 mg, 6.7 mg, 6.8 mg, 6.9 mg, 7.0 mg, 7.1 mg, 7.2 mg, 7.3 mg, 7.4 mg, 7.5 mg, 7.6 mg, 7.7 mg, 7.8 mg, 7.9 mg, 8.0 mg, 8.1 mg, 8.2 mg, 8.3 mg, 8.4 mg, 8.5 mg, 8.6 mg, 8.7 mg, 8.8 mg, 8.9 mg, 9.0 mg, 9.1 mg, 9.2 mg, 9.3 mg, 9.4 mg, 9.5 mg, 9.6 mg, 9.7 mg, 9.8 mg, 9.9 mg, 10.0 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 55 mg, 60 mg, 65 mg, 70 mg, 75 mg, 80 mg, 85 mg, 90 mg, 95 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1 g, 2 g, 3 g, 4 g, 5 g, 6 g, 7 g, 8 g, 9 g, 10 g or within a range defined by, and including, any two of these values.

The following examples are given to illustrate various embodiments of the present invention in the field of DNA immunization, which can be delivered to a subject in need of an immune response to the antigen contained therein. It is to be understood that the following examples are not comprehensive or exhaustive of the many types of embodiments which can be prepared in accordance with the present invention.

### Example 1

The NS3/4A sequence was amplified from the serum of an HCV-infected patient (HCV genotype 1a) using the Polymerase Chain Reaction (PCR). Total RNA was extracted from serum, and cDNA synthesis and PCR were performed according to standard protocols (Chen M et al., J. Med. Virol. 43:223-226 (1995)). The cDNA synthesis was initiated using the antisense primer “NS4KR” (5′-CCG TCT AGA TCA GCA CTC TTC CAT TTC ATC-3′ (SEQ. ID. NO. 98)). From this cDNA, a 2079 base pair DNA fragment of HCV, corresponding to amino acids 1007 to 1711, which encompasses the NS3 and NS4A genes, was amplified. A high fidelity polymerase (Expand High Fidelity PCR, Boehringer-Mannheim, Mannheim, Germany) was used with the “NS3KF” primer (5′-CCT GAA TTC ATG GCG CCT ATC ACG GCC TAT-3′ (SEQ. ID. NO. 99) and the NS4KR primer. The NS3KF primer contained a EcoRI restriction enzyme cleavage site and a start codon and the primer NS4KR contained a XbaI restriction enzyme cleavage site and a stop codon.

The amplified fragment was then sequenced (SEQ. ID. NO. 100). Sequence comparison analysis revealed that the gene fragment was amplified from a viral strain of genotype 1a. A computerized BLAST search against the Genbank database using the NCBI website revealed that the closest HCV homologue was 93% identical in nucleotide sequence.

The amplified DNA fragment was then digested with EcoRI and XbaI, and was inserted into a pcDNA3.1/His plasmid (Invitrogen) digested with the same enzymes. The NS3/4A-pcDNA3.1 plasmid was then digested with EcoRI and Xba I and the insert was purified using the QiaQuick kit (Qiagen, Hamburg, Germany) and was ligated to a EcoRI/Xba I digested pVAX vector (Invitrogen) so as to generate the NS3/4A-pVAX plasmid.

The NS3 truncated mutant was obtained by deleting NS4A sequence from the NS3/4A DNA. Accordingly, the NS3 gene sequence of NS3/4A-pVAX was PCR amplified using the primers NS3KF and 3′ NotI (5′-CCA CGC GGC CGC GAC GAC CTA CAG-3′ (SEQ. ID. NO.: 101)) containing EcoRI and Not I restriction sites, respectively. The NS3 fragment (1850 bp) was then ligated to a EcoRI and Not I digested pVAX plasmid to generate the NS3-pVAX vector. Plasmids were grown in BL21 E. coli cells. The plasmids were sequenced and were verified by restriction cleavage and the results were as to be expected based on the original sequence.

### Example 2

To assess the ability of HBcAg DNA constructs to prime CTLs, the nucleic acid of SEQ ID NO:10 is cloned into the pVAX1 expression vector (Invitrogen, Carlsbad, Calif.) to create HBcAg-pVAX1.

Plasmids are grown in BL21 E. coli cells, and sequenced for accuracy. Plasmid DNA used for in vivo vaccination is purified using Qiagen DNA purification columns, according to the manufacturer's instructions (Qiagen GmbH, Hilden, FRG).

Groups of eight to ten C57/BL6 mice are primed with HBcAg-pVAX1 intra muscularly (i.m.). For i.m. delivery, mice are immunized by needle injections of 100 plasmid DNA given intramuscularly to the tibialis anterior (TA) muscle. 5 days prior to DNA immunization, mice are injected intramuscularly with 50 μl per TA muscle of 0.01 mM cardiotoxin (Latoxan) in 0.9% sterile saline. The mice are boosted with a second injection of 100 μg plasmid DNA four weeks subsequent to the first DNA immunization. Each injection dose contains 100 μg of plasmid DNA. Immunizations are performed at weeks 0 and 4.

The presence of CTLs specific for SEQ ID NO:10 is assayed using a standard ⁵¹Cr-release assay. Briefly, spleen cells are harvested from mice 14 days after the initial immunization or the booster immunization. Chromium release assays are performed as described in Lazdina, et al. (2003) J. Gen. Virol. 84:1-8, herein expressly incorporated by reference in its entirety. Single cell suspensions are prepared. 25×10⁶ splenocytes are restimulated with 25×10⁶ syngenic irradiated (20 Gy) splenocytes pulsed with 0.05 μM peptide, as previously described. Sandberg et al. (2000) J. Immunol. 165:25-33, herein expressly incorporated by reference in its entirety. Restimulation cultures are set in 12 ml complete RPMI medium (Gibco). After 5 days, effector cells are harvested and washed twice. RMA-S target cells (Karre et al. (1986) Nature 319:675-678) are pulsed with 50 μM peptide for 90 min at 5% CO₂ and 37° C. Serial dilutions of effector cells are incubated with 5×10^{3 51}chromium-labeled peptide pulsed RMA-S target cells in a final volume of 200 μl per well in 96-well plates. After a 4 hour incubation at 5% CO₂ and 37° C., 100 μl of supernatant is collected and the radioactivity is determined using a γ counter. The percentage of specific release is calculated according to the formula: (Experimental release−spontaneous release/total release−spontaneous release)×100.

### Example 3

The expression of the HBcAg and NS3/4a proteins from plasmids were analyzed by an in vitro transcription and translation assay. Each sequence was cloned into pVAX1 expression vector (Invitrogen, Carlsbad, Calif.).

The following constructs were studied: (1) codon-optimized NS3/4A (SEQ. ID. No. 2); (2) codon-optimized HBcAg; (3) NS3/4A-HBcAg (SEQ. ID. No. 73); (4) mutant NS3/4A-HBcAg (SEQ. ID. No. 75); (5) N53-NS4A/B junction-N54-HBcAg (SEQ. ID. No. 77) (6) N53-NS4A/B junction-NS4-NS4A/B junction-HBcAg (SEQ. ID. No. 79); and (5-11) NS3/4A-NS4A/B junction-HBcAg fragments (SEQ. ID Nos. 81, 83, 85, 87 and 89, respectively) (hereinafter Constructs 1-11, respectively).

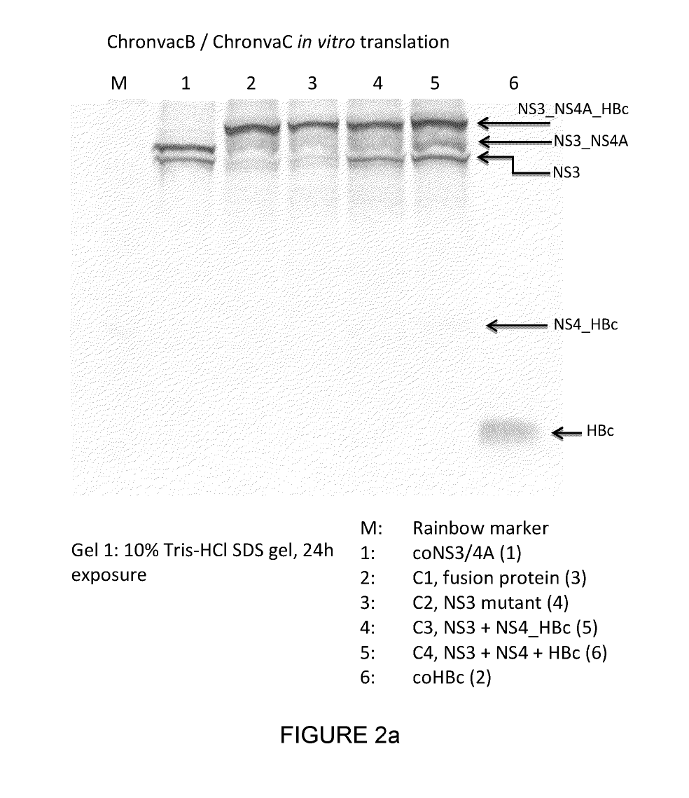

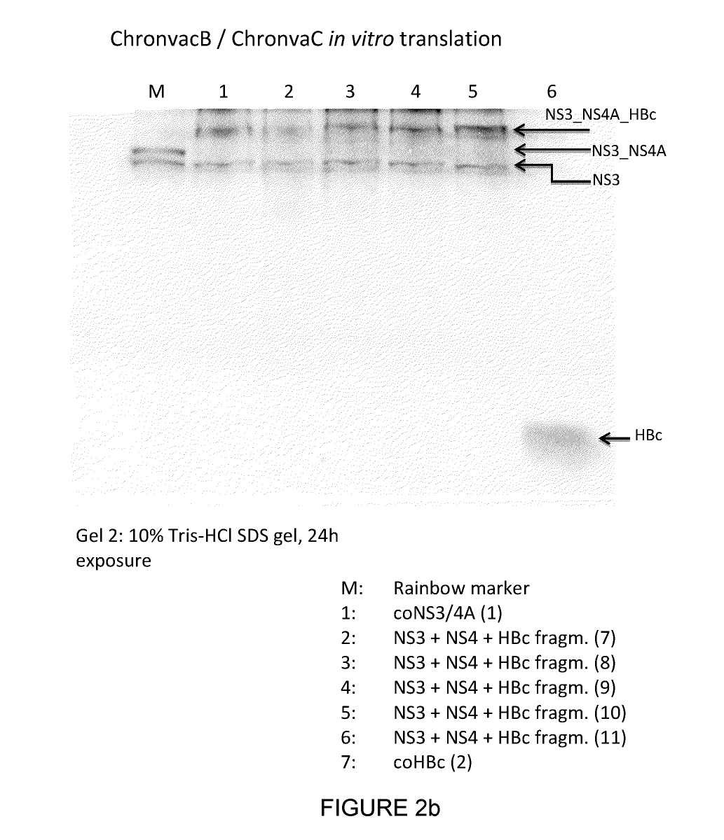

FIGS. 2a-b show the results of gel electrophoresis using 10% Tris-HCl SDS gel after 24 hours of exposure. The results confirm that constructs encoding cleavage sites were cleaved to form multiple, distinct proteins. For example, Construct 4 exhibits 2 sharp bands associated with two portions of the encoded polypeptide that are separated by a cleavage site. In contrast, nucleic acids lacking cleavage sites, such as Construct 2, exhibit only a single sharp band.

### Example 4

Constructs 1 and 4, as discussed in Example 3, were tested in mouse models to assay the ability to induce and immune response. Plasmids were grown in BL21 E. coli cells, and sequenced for accuracy. Plasmid DNA used for in vivo vaccination was purified using Qiagen DNA purification columns, according to the manufacturer's instructions (Qiagen GmbH, Hilden, FRG). The concentration of the resulting plasmid DNA was determined spectrophotometrically (Dynaquant, Pharmacia Biotech, Uppsala, Sweden) and the purified DNA was dissolved in sterile phosphate buffered saline (PBS) at a concentration of 1 mg/ml.

Two types of mice were tested, HLA-A2 transgenic mice (HHD) and HCV NS3/4A+HLA-A2 transgenic mice (H3). The HCV NS3/4A+HLA-A2 transgenic mouse model is a preferred animal model for therapeutic vaccination because it provides a partly human immune system that is dysfunctional due to a persistent presence of a viral antigen. Accordingly, this model is representative of chronic HCV infection in humans.

Mice were intra muscularly (i.m.) immunized with 50 μg of Construct 1 or 4 at 0 and 4 weeks. Meanwhile, four other mice groups were co-administered 50 μg of IL-12 or IL-21 along with Construct 1 or 4 at 0 and 4 weeks. Mice were sacrificed at week 6 and spleens harvested and analyzed for HCV-specific IFNγ production by ELISpot as described in Ahlen G, Soderholm J, Tjelle T E, et al. “In vivo Electroporation Enhances the Immunogenicity of Hepatitis C Virus Nonstructural3/4A DNA by Increased Local DNA Uptake, Protein Expression, Inflammation, and Infiltration of CD3+ cells,” J. Immunol. (2007), which is hereby incorporated by reference in its entirety. Table 3 provided below shows a list of restricted peptides in the transgenic mice whose expression was detected using ELISpot.

TABLE 3

| | | SEQ. |
|---|---|---|
| | RESTRICTED | ID. |
| IDENTIFIER | SEQUENCE | NO. |
| TP-5 | GLLGCIITSL | 90 |
| TP-6 | TGSPITYSTY | 91 |
| TP-7 | KLVALGVNAV | 92 |
| TP-9 | CINGVCWTV | 93 |
| TP-10 | LLCPAGHAV | 94 |
| TP-11 | ATMGFGAYM | 95 |
| TP-12 | YLVAYQATV | 96 |
| TP-13 | TLHGPTPLL | 97 |

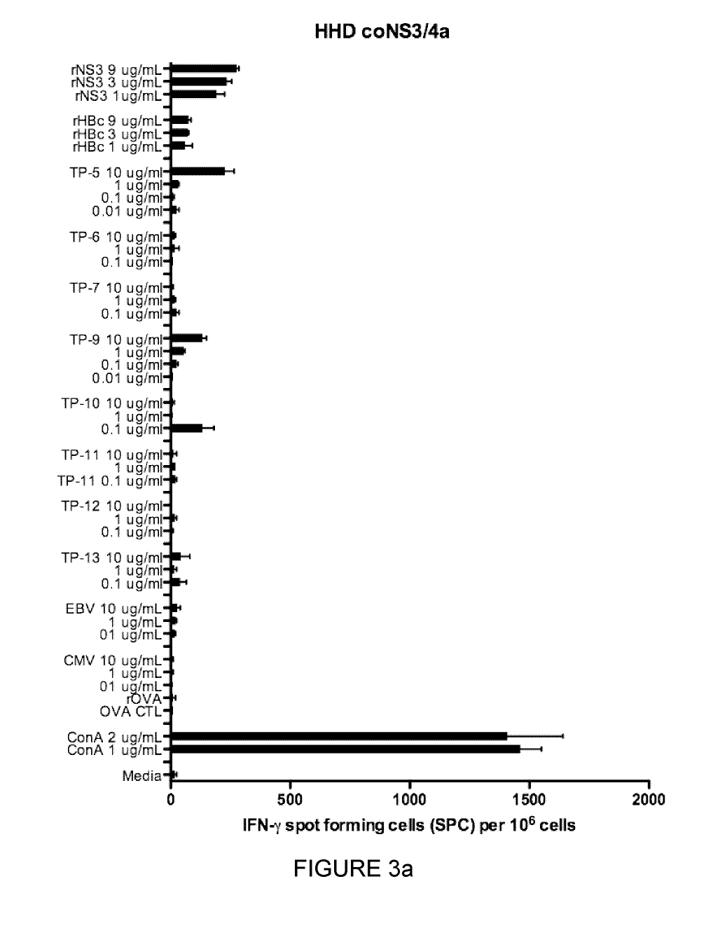

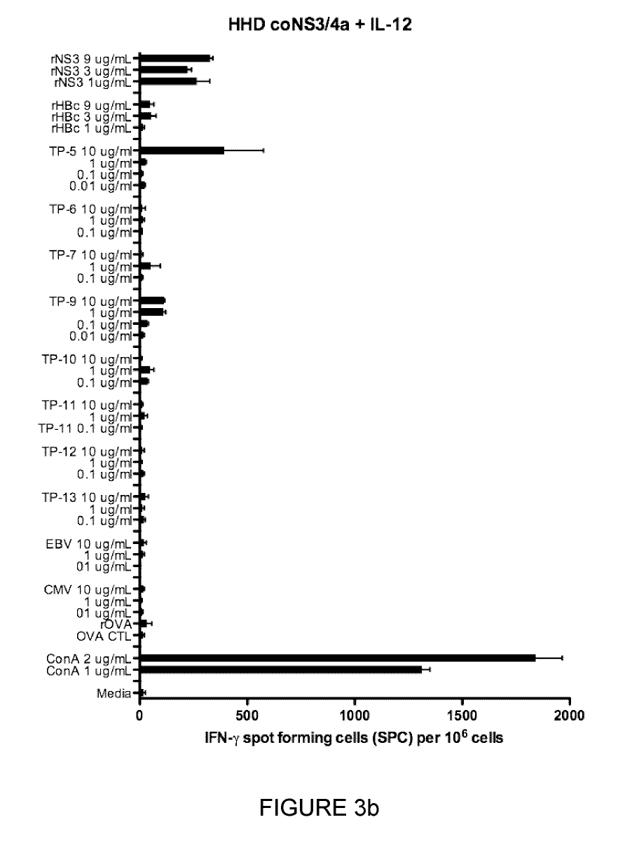

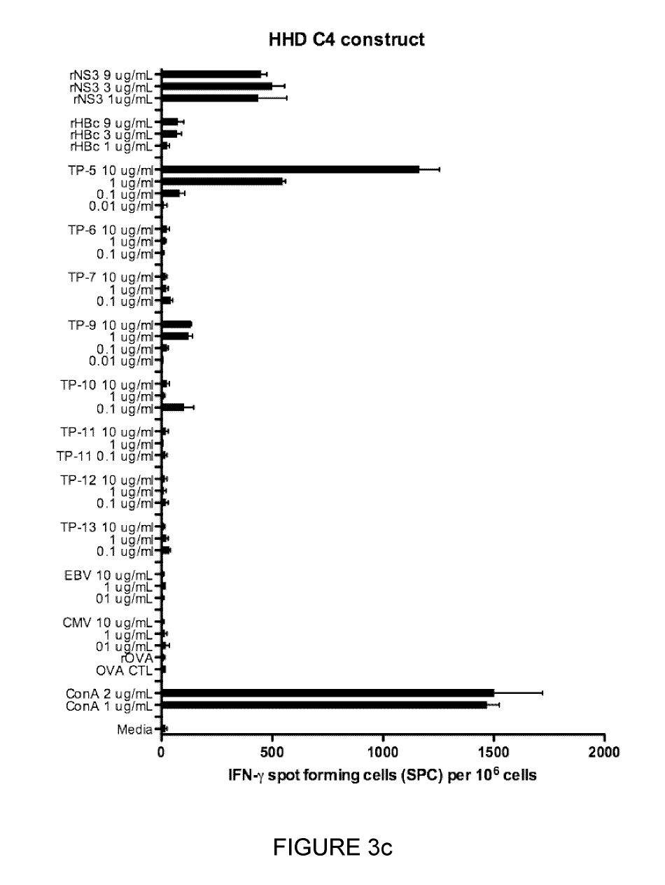

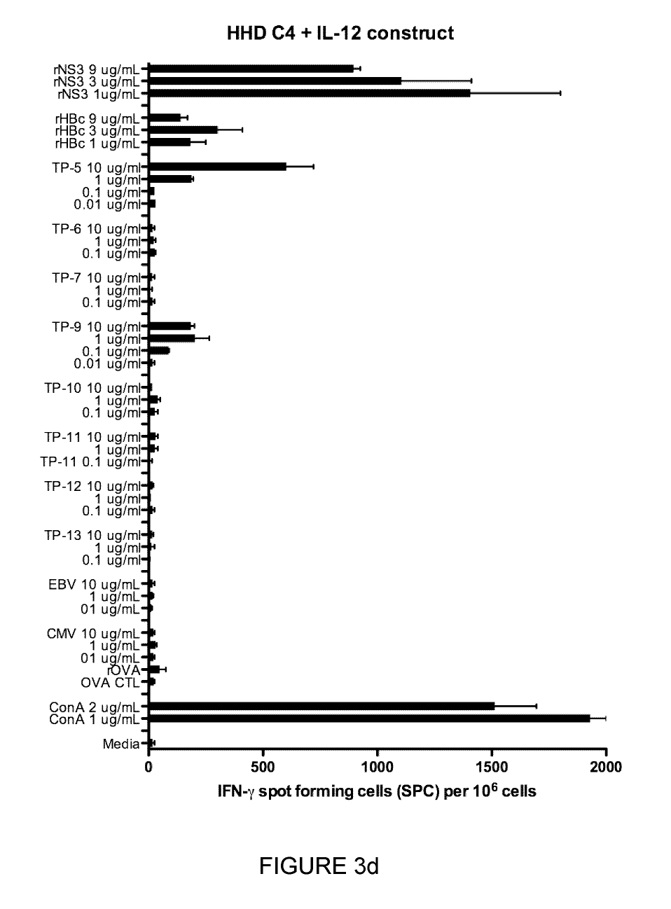

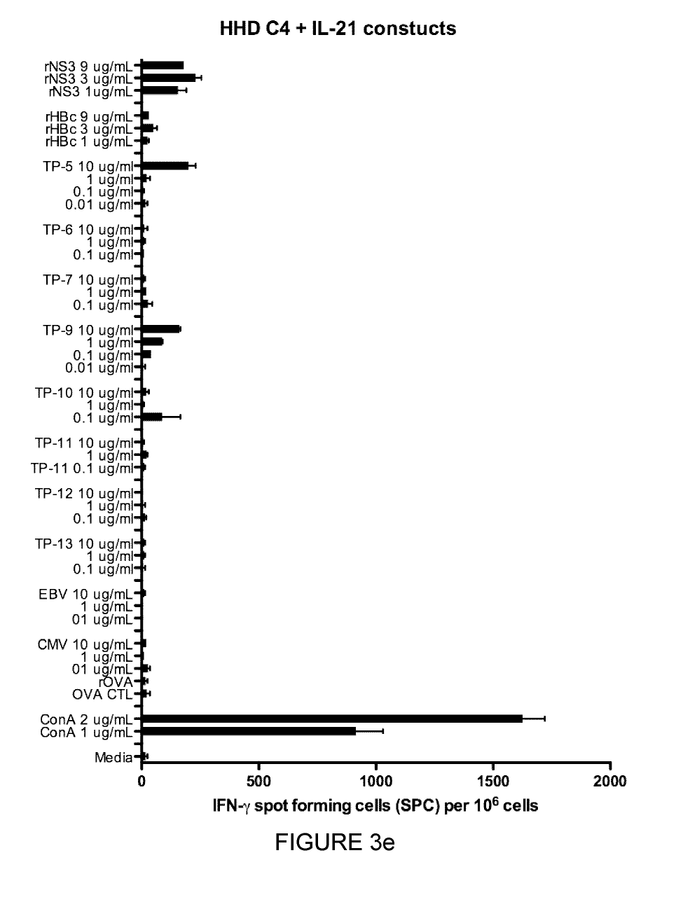

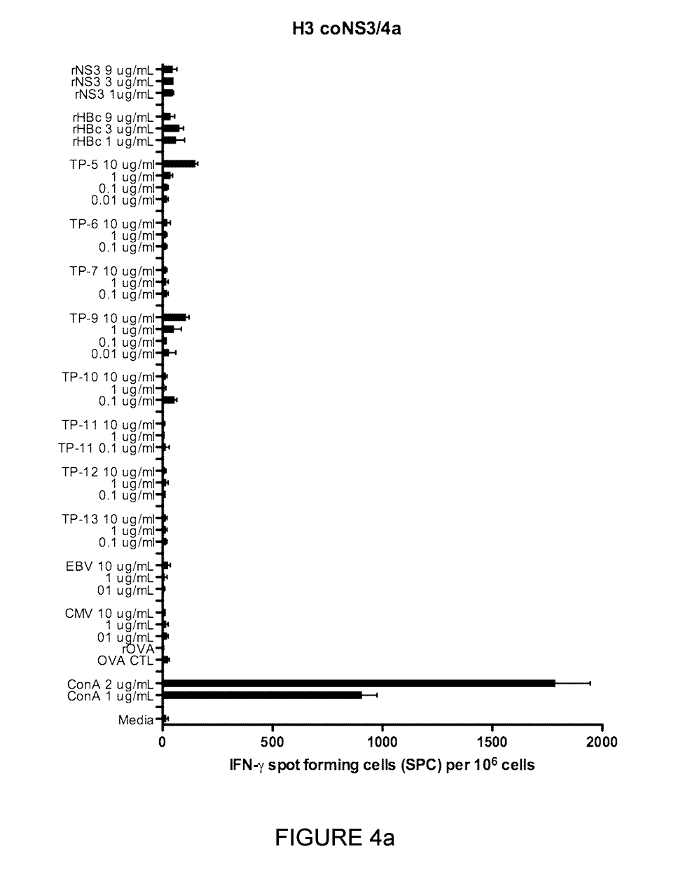

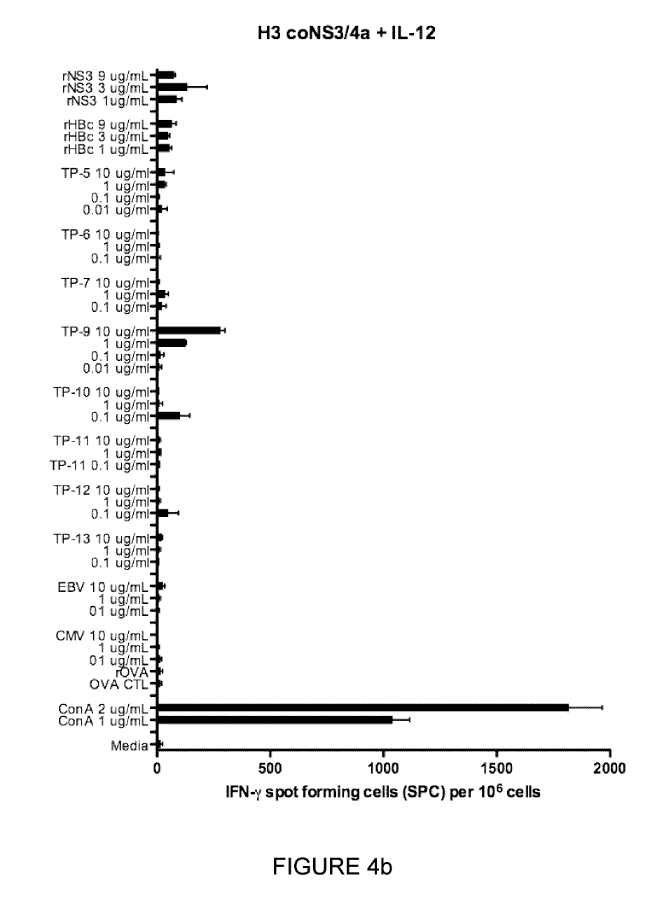

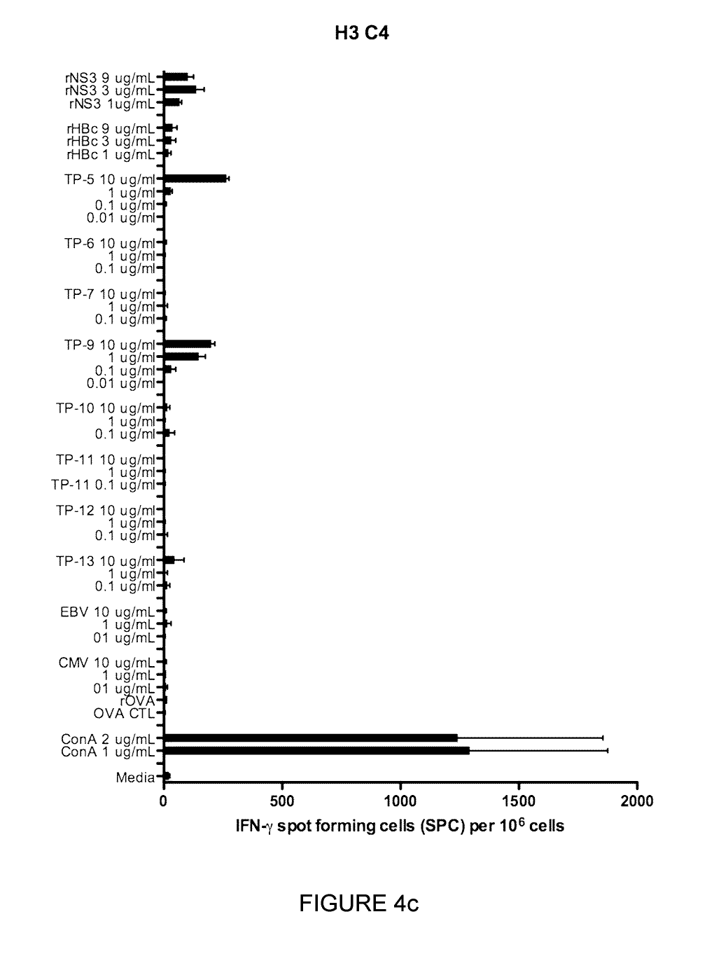

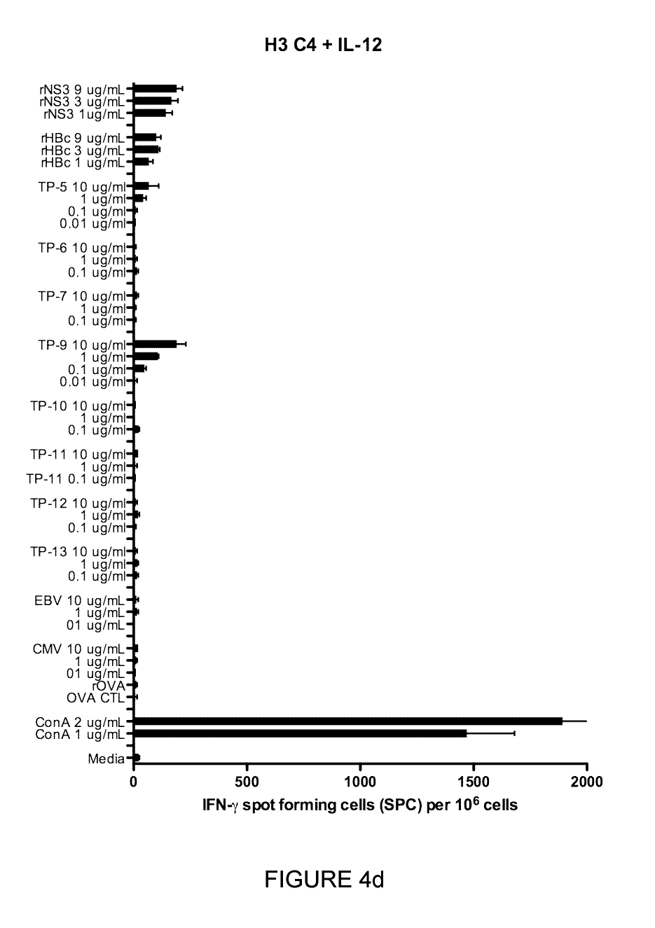

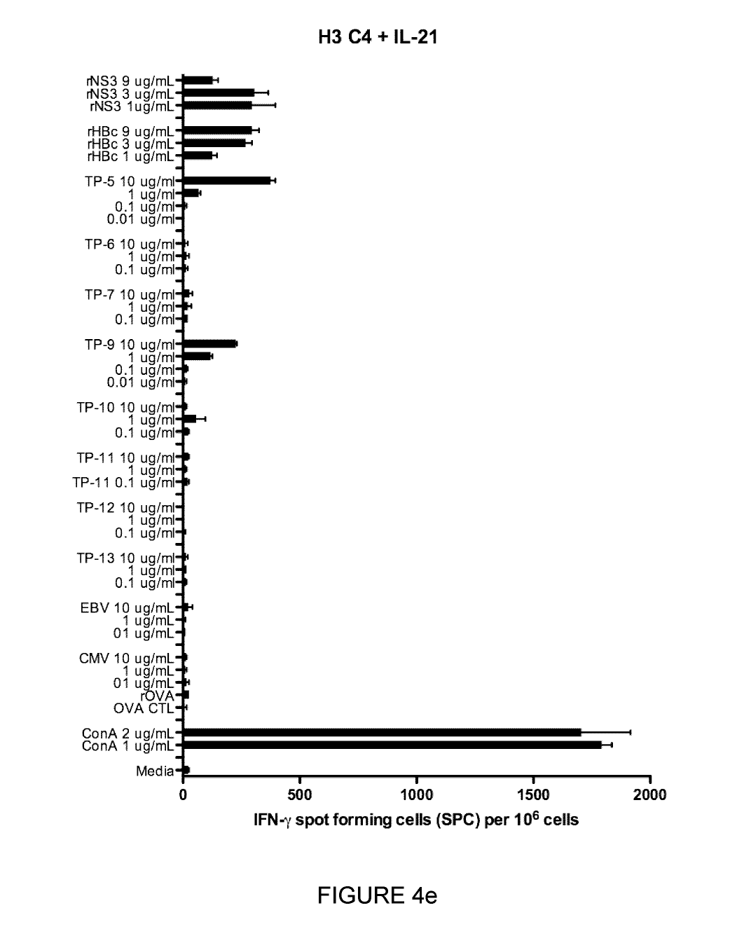

ELISpot results are shown in FIGS. 3a-e and 4a-e for the HHD and H3 animal models, respectively. More specifically, FIG. 3a-c shows the immune response from the administration of codon-optimized NS3/4A (Construct 1), codon-optimized NS3/4A coadministered with IL-12, and mutant NS3/4A-HBcAg (Construct 4), respectively, when administered to HHD mice. The adjuvant activity of HBcAg is demonstrated by the increased immune response of mice receiving Construct 4 relative to both Construct 1 and Construct 1 co-administered with IL-12. FIGS. 4a-c show the immune response from the administration of codon-optimized NS3/4A (Construct 1), codon-optimized NS3/4A coadministered with IL-12, and mutant NS3/4A-HBcAg (Construct 4), respectively, when administered to H3 mice. These results further demonstrate the adjuvant activity of HBcAg.

To further improve the immune response, mutant NS3/4A-HBcAg was co-administered with either IL-12 or IL-21 to HHD and H3 mice. FIGS. 3d-e show results in the HHD mouse model, and demonstrate the immune response is further increased by the addition of IL-12 or IL-21, relative to mutant NS3/4A-HBcAg administered alone (i.e., as shown in FIG. 3c). The results show IL-12 produced generally a greater immune response compared to IL-21. Finally, FIGS. 4d-e show the results in the H3 mouse model. Again, both IL-12 and IL-21 improved the immune response of mutant NS3/4A-HBcAg relative the administration of mutant NS3/4A-HBcAg alone (i.e., as shown in FIG. 4c). Most interestingly, IL-21 produced a generally greater immune response in H3 mouse compared to IL-12.

### Examples 4-13

To further evaluate the adjuvant activity of HBcAg, both HHD and H3 transgenic mice are instramuscularly administered compositions having constructs encoding HBcAg and isolated constructs encoding an antigen. To prepare each construct, each sequence is independently cloned into a separate pVAX1 expression vector (Invitrogen, Carlsbad, Calif.). The plasmids are prepared generally using the same techniques as disclosed in Example 2.

Compositions are prepared by admixing a vector encoding codon-optimized HBcAg and a vector encoding an antigen in sterile phosphate buffered saline (PBS) at a concentration of 1 mg/ml. 50 μg of this mixture is administered intramuscularly to HHD and H3 mice using the same techniques and analyzed using ELISpot as described in Example 3. These results are compared to mice receiving antigen but without co-administered HBcAg.

Table 4 below lists the specific nucleic acids inserted into vectors and contained in the admixtures administered for Examples 4-13. Thus, for example, Example 4 includes the administration of a vector encoding codon-optimized stork HBcAg, and a vector encoding codon-optimized NS3/4A.

TABLE 4

| EXAMPLE | HBcAg (SEQ. ID. No.) | ANTIGEN (SEQ. ID. NO.) |
|---|---|---|
| 4 | 20 | 2 |
| 5 | 22 | 2 |
| 6 | 20 | 8 |
| 7 | 22 | 8 |
| 8 | 20 | 10 |
| 9 | 22 | 10 |
| 10 | 20 | 12 |
| 11 | 22 | 12 |
| 12 | 20 | 16 |
| 13 | 22 | 16 |
| 14 | 20 | 18 |
| 15 | 22 | 18 |

It will be shown that the presence of HBcAg in the composition promotes a more robust immune response to the antigen in the subject, as compared to administration of a composition of antigen that excludes effective amounts of HBcAg.

### Examples 14-43

Additional experiments to study the immunogenic properties of isolated nucleic acids encoding HBcAg joined to a heterologous protein can be performed. The procedures are generally the same as those described in Example 4, which briefly includes inserting the sequence into the pVAX1 plasmid and administering a composition of the plasmid to HHD and H3 transgenic mice. The immune response is determined using ELISpot and compared to the immune response resulting from administering plasmids encoding the antigen without HBcAg. The nucleic acids used in Examples 14-43 are shown below in Table 5.

TABLE 5

| | | | NUCLEIC |
|---|---|---|---|
| | NUCLEIC ACID | | ACID (SEQ. ID. |
| EXAMPLE | (SEQ. ID. NO.) | EXAMPLE | NO.) |
| 14 | 24 | 29 | 54 |
| 15 | 26 | 30 | 56 |
| 16 | 28 | 31 | 58 |
| 17 | 30 | 32 | 60 |
| 18 | 32 | 33 | 62 |
| 19 | 34 | 34 | 64 |
| 20 | 36 | 35 | 66 |
| 21 | 38 | 36 | 68 |
| 22 | 40 | 37 | 81 |
| 23 | 42 | 38 | 83 |
| 24 | 44 | 49 | 85 |
| 25 | 46 | 40 | 87 |
| 26 | 48 | 41 | 89 |
| 27 | 50 | 42 | 103 |
| 28 | 52 | 43 | 105 |

It will be shown that the compositions having HBcAg joined to an antigen promote a more robust immune response to the antigen in the subject, as compared to administration of a composition of antigen that excludes effective amounts of HBcAg joined to the antigen.

### Example 44

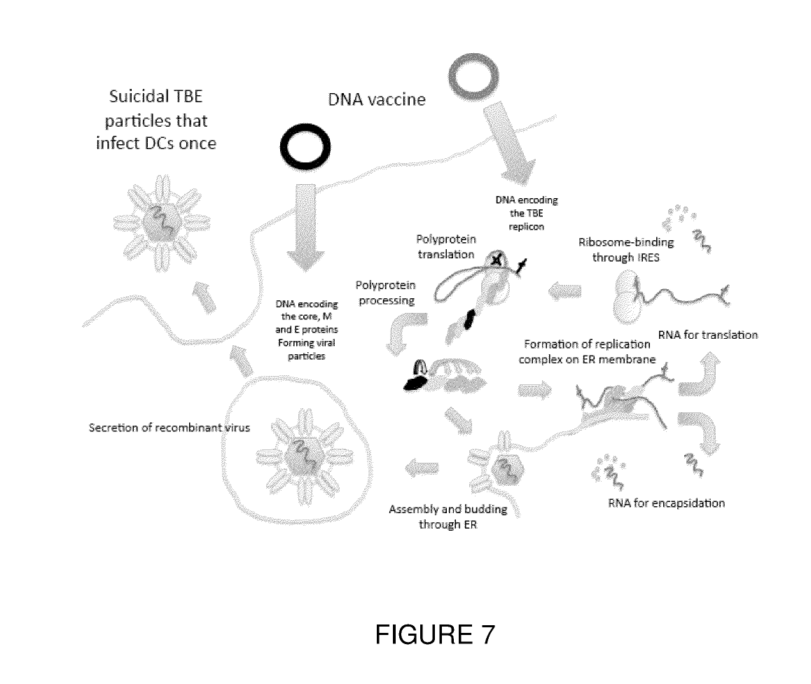

As illustrated in FIG. 7, a DNA vaccine using a flavivirus replicon, for example a tick-borne encephalitis (TBE) replicon, is described in this example. Muscle or skin cells are introduced (e.g. by transfection or injection) with at least two DNA plasmids, one plasmid expressing the flaviviral/TBE envelope proteins and at least one plasmid expressing the flavivirus/TBE replicon encoding the non-structural viral proteins and a gene of interest which may include but is not limited to any of the nucleotide sequences disclosed herein that may serve as an immunogen (e.g. HCV NS3/4A and/or HBcAg/HHcAg/SHcAg). The cells expressing the plasmids produce flaviviral or TBE particles that can infect once and produce new non-structural proteins (replicon) and the gene of interest. Because the flaviviral particles infect once, they are replication defective or “suicidal.” The replication defective/suicidal virus particles containing the replicon RNA target and infect professional antigen presenting cells such as dendritic cells or Langerhan cells, delivering the replicon RNA. Within the infected dendritic or Langerhan cells, the flaviviral or TBE replicon RNA is replicated and the non-structural proteins and gene of interest (e.g. HCV NS3/4A and/or HBcAg/HHcAg/SHcAg) are amplified, thereby activating T-cells (e.g. Th1 and CTL) directed against the gene of interest.

### Example 45

The DNA vaccine(s) described in EXAMPLE 44 are delivered to a subject by intramuscular injection in the tibialis anterior muscle. Similarly, reporter replicons in which the gene of interest encodes for a reporter such as luciferase or green fluorescent protein are delivered to a subject by intramuscular injection in the tibialis anterior muscle. The biodistribution of plasmid DNA and replicon RNA is determined by PCR. Reporter gene expression in vivo is determined by immunohistochemistry, western blot and in vivo imaging. The kinetics of the plasmid in the muscle, the kinetics of replicon RNA and expression of the gene of interest are characterized.

### Example 46

The immune responses to the vector itself as well as the gene of interest is determined by in vivo and in vitro techniques. The dynamics of the appearance of specific T cells is determined by ELIspot assays as well as a direct quantitation of specific T cells by flow cytometry. The in vivo functionality is tested in several models including stable and transiently transgenic mice after vaccination.

What is claimed:

1. An immunogenic composition comprising:
(a) a first construct that comprises a nucleic acid sequence encoding a tick-borne encephalitis (TBE) core, Pre-M, and envelope proteins, but lacking the TBE non-structural replicon proteins; and
(b) a second construct that comprises a nucleic acid sequence encoding a hepatitis C virus (HCV) NS3/4A fusion protein and TBE non-structural replicon proteins.
